SEQUENCE LISTING (147 sequences)

SEQ ID NO: 1 - protein, 857 aa, Homo sapiens
MEAGPSGAAAGAYLPPLQQVFQAPRRPGIGTVGKPIKLLANYFEVDIPKIDVYHYEVDIKPDKCPRRVNREVVEYMVQHFKPQIFGDRKPVYDGKKNIYTVTALPIGNERVDFEVTIPGEGKDRIFKVSIKWLAIVSWRMLHEALVSGQIPVPLESVQALDVAMRHLASMRYTPVGRSFFSPPEGYYHPLGGGREVWFGFHQSVRPAMWKMMLNIDVSATAFYKAQPVIEFMCEVLDIRNIDEQPKPLTDSQRVRFTKEIKGLKVEVTHCGQMKRKYRVCNVTRRPASHQTFPLQLESGQTVECTVAQYFKQKYNLQLKYPHLPCLQVGQEQKHTYLPLEVCNIVAGQRCIKKLTDNQTSTMIKATARSAPDRQEEISRLMKNASYNLDPYIQEFGIKVKDDMTEVTGRVLPAPILQYGGRNRAIATPNQGVWDMRGKQFYNGIEIKVWAIACFAPQKQCREEVLKNFTDQLRKISKDAGMPIQGQPCFCKYAQGADSVEPMFRHLKNTYSGLQLIIVILPGKTPVYAEVKRVGDTLLGMATQCVQVKNVVKTSPQTLSNLCLKINVKLGGINNILVPHQRSAVFQQPVIFLGADVTHPPAGDGKKPSITAVVGSMDAHPSRYCATVRVQRPRQEIIEDLSYMVRELLIQFYKSTRFKPTRIIFYRDGVPEGQLPQILHYELLAIRDACIKLEKDYQPGITYIVVQKRHHTRLFCADKNERIGKSGNIPAGTTVDTNITHPFEFDFYLCSHAGIQGTSRPSHYYVLWDDNRFTADELQILTYQLCHTYVRCTRSVSIPAPAYYARLVAFRARYHLVDKEHDSGEGSHISGQSNGRDPQALAKAVQVHQDTLRTMYFA

SEQ ID NO: 2 - protein, 859 aa, Homo sapiens
MYSGAGPALAPPAPPPPIQGYAFKPPPRPDFGTSGRTIKLQANFFEMDIPKIDIYHYELDIKPEKCPRRVNREIVEHMVQHFKTQIFGDRKPVFDGRKNLYTAMPLPIGRDKVELEVTLPGEGKDRIFKVSIKWVSCVSLQALHDALSGRLPSVPFETIQALDVVMRHLPSMRYTPVGRSFFTASEGCSNPLGGGREVWFGFHQSVRPSLWKMMLNIDVSATAFYKAQPVIEFVCEVLDFKSIEEQQKPLTDSQRVKFTKEIKGLKVEITHCGQMKRKYRVCNVTRRPASHQTFPLQQESGQTVECTVAQYFKDRHKLVLRYPHLPCLQVGQEQKHTYLPLEVCNIVAGQRCIKKLTDNQTSTMIRATARSAPDRQEEISKLMRSASFNTDPYVREFGIMVKDEMTDVTGRVLQPPSILYGGRNKAIATPVQGVWDMRNKQFHTGIEIKVWAIACFAPQRQCTEVHLKSFTEQLRKISRDAGMPIQGQPCFCKYAQGADSVEPMFRHLKNTYAGLQLVVVILPGKTPVYAEVKRVGDTVLGMATQCVQMKNVQRTTPQTLSNLCLKINVKLGGVNNILLPQGRPPVFQQPVIFLGADVTHPPAGDGKKPSIAAVVGSMDAHPNRYCATVRVQQHRQEIIQDLAAMVRELLIQFYKSTRFKPTRIIFYRDGVSEGQFQQVLHHELLAIREACIKLEKDYQPGITFIVVQKRHHTRLFCTDKNERVGKSGNIPAGTTVDTKITHPTEFDFYLCSHAGIQGTSRPSHYHVLWDDNRFSSDELQILTYQLCHTYVRCTRSVSIPAPAYYAHLVAFRARYHLVDKEHDSAEGSHTSGQSNGRDHQALAKAVQVHQDTLRTMYFA

SEQ ID NO: 3 - protein, 860 aa, Homo sapiens
MEIGSAGPAGAQPLLMVPRRPGYGTMGKPIKLLANCFQVEIPKIDVYLYEVDIKPDKCPRRVNREVVDSMVQHFKVTIFGDRRPVYDGKRSLYTANPLPVATTGVDLDVTLPGEGGKDRPFKVSIKFVSRVSWHLLHEVLTGRTLPEPLELDKPISTNPVHAVDVVLRHLPSMKYTPVGRSFFSAPEGYDHPLGGGREVWFGFHQSVRPAMWKMMLNIDVSATAFYKAQPVIQFMCEVLDIHNIDEQPRPLTDSHRVKFTKEIKGLKVEVTHCGTMRRKYRVCNVTRRPASHQTFPLQLENGQTVERTVAQYFREKYTLQLKYPHLPCLQVGQEQKHTYLPLEVCNIVAGQRCIKKLTDNQTSTMIKATARSAPDRQEEISRLVRSANYETDPFVQEFQFKVRDEMAHVTGRVLPAPMLQYGGRNRTVATPSHGVWDMRGKQFHTGVEIKMWAIACFATQRQCREEILKGFTDQLRKISKDAGMPIQGQPCFCKYAQGADSVEPMFRHLKNTYSGLQLIIVILPGKTPVYAEVKRVGDTLLGMATQCVQVKNVIKTSPQTLSNLCLKINVKLGGINNILVPHQRPSVFQQPVIFLGADVTHPPAGDGKKPSIAAVVGSMDAHPSRYCATVRVQRPRQEIIQDLASMVRELLIQFYKSTRFKPTRIIFYRDGVSEGQFRQVLYYELLAIREACISLEKDYQPGITYIVVQKRHHTRLFCADRTERVGRSGNIPAGTTVDTDITHPYEFDFYLCSHAGIQGTSRPSHYHVLWDDNCFTADELQLLTYQLCHTYVRCTRSVSIPAPAYYAHLVAFRARYHLVDKEHDSAEGSHVSGQSNGRDPQALAKAVQIHQDTLRTMYFA

SEQ ID NO: 4 - protein, 861 aa, Homo sapiens
MEALGPGPPASLFQPPRRPGLGTVGKPIRLLANHFQVQIPKIDVYHYDVDIKPEKRPRRVNREVVDTMVRHFKMQIFGDRQPGYDGKRNMYTAHPLPIGRDRVDMEVTLPGEGKDQTFKVSVQWVSVVSLQLLLEALAGHLNEVPDDSVQALDVITRHLPSMRYTPVGRSFFSPPEGYYHPLGGGREVWFGFHQSVRPAMWNMMLNIDVSATAFYRAQPIIEFMCEVLDIQNINEQTKPLTDSQRVKFTKEIRGLKVEVTHCGQMKRKYRVCNVTRRPASHQTFPLQLENGQAMECTVAQYFKQKYSLQLKYPHLPCLQVGQEQKHTYLPLEVCNIVAGQRCIKKLTDNQTSTMIKATARSAPDRQEEISRLVKSNSMVGGPDPYLKEFGIVVHNEMTELTGRVLPAPMLQYGGRNKTVATPNQGVWDMRGKQFYAGIEIKVWAVACFAPQKQCREDLLKSFTDQLRKISKDAGMPIQGQPCFCKYAQGADSVEPMFKHLKMTYVGLQLIVVILPGKTPVYAEVKRVGDTLLGMATQCVQVKNVVKTSPQTLSNLCLKINAKLGGINNVLVPHQRPSVFQQPVIFLGADVTHPPAGDGKKPSIAAVVGSMDGHPSRYCATVRVQTSRQEISQELLYSQEVIQDLTNMVRELLIQFYKSTRFKPTRIIYYRGGVSEGQMKQVAWPELIAIRKACISLEEDYRPGITYIVVQKRHHTRLFCADKTERVGKSGNVPAGTTVDSTITHPSEFDFYLCSHAGIQGTSRPSHYQVLWDDNCFTADELQLLTYQLCHTYVRCTRSVSIPAPAYYARLVAFRARYHLVDKDHDSAEGSHVSGQSNGRDPQALAKAVQIHHDTQHTMYFA

SEQ ID NO: 5 - protein, 1226 aa, Homo sapiens
MNPRQGYSLSGYYTHPFQGYEHRQLRYQQPGPGSSPSSFLLKQIEFLKGQLPEAPVIGKQTPSLPPSLPGLRPRFPVLLASSTRGRQVDIRGVPRGVHLRSQGLQRGFQHPSPRGRSLPQRGVDCLSSHFQELSIYQDQEQRILKFLEELGEGKATTAHDLSGKLGTPKKEINRVLYSLAKKGKLQKEAGTPPLWKIAVSTQAWNQHSGVVRPDGHSQGAPNSDPSLEPEDRNSTSVSEDLLEPFIAVSAQAWNQHSGVVRPDSHSQGSPNSDPGLEPEDSNSTSALEDPLEFLDMAEIKEKICDYLFNVSDSSALNLAKNIGLTKARDINAVLIDMERQGDVYRQGTTPPIWHLTDKKRERMQIKRNTNSVPETAPAAIPETKRNAEFLTCNIPTSNASNNMVTTEKVENGQEPVIKLENRQEARPEPARLKPPVHYNGPSKAGYVDFENGQWATDDIPDDLNSIRAAPGEFRAIMEMPSFYSHGLPRCSPYKKLTECQLKNPISGLLEYAQFASQTCEFNMIEQSGPPHEPRFKFQVVINGREFPPAEAGSKKVAKQDAAMKAMTILLEEAKAKDSGKSEESSHYSTEKESEKTAESQTPTPSATSFFSGKSPVTTLLECMHKLGNSCEFRLLSKEGPAHEPKFQYCVAVGAQTFPSVSAPSKKVAKQMAAEEAMKALHGEATNSMASDNQPEGMISESLDNLESMMPNKVRKIGELVRYLNTNPVGGLLEYARSHGFAAEFKLVDQSGPPHEPKFVYQAKVGGRWFPAVCAHSKKQGKQEAADAALRVLIGENEKAERMGFTEVTPVTGASLRRTMLLLSRSPEAQPKTLPLTGSTFHDQIAMLSHRCFNTLTNSFQPSLLGRKILAAIIMKKDSEDMGVVVSLGTGNRCVKGDSLSLKGETVNDCHAEIISRRGFIRFLYSELMKYNSQTAKDSIFEPAKGGEKLQIKKTVSFHLYISTAPCGDGALFDKSCSDRAMESTESRHYPVFENPKQGKLRTKVENGEGTIPVESSDIVPTWDGIRLGERLRTMSCSDKILRWNVLGLQGALLTHFLQPIYLKSVTLGYLFSQGHLTRAICCRVTRDGSAFEDGLRHPFIVNHPKVGRVSIYDSKRQSGKTKETSVNWCLADGYDLEILDGTRGTVDGPRNELSRVSKKNIFLLFKKLCSFRYRRDLLRLSYGEAKKAARDYETAKNYFKKGLKDMGYGNWISKPQEEKNFYLCPV

SEQ ID NO: 6 - protein, 701 aa, Homo sapiens
MDIEDEENMSSSSTDVKENRNLDNVSPKDGSTPGPGEGSQLSNGGGGGPGRKRPLEEGSNGHSKYRLKKRRKTPGPVLPKNALMQLNEIKPGLQYTLLSQTGPVHAPLFVMSVEVNGQVFEGSGPTKKKAKLHAAEKALRSFVQFPNASEAHLAMGRTLSVNTDFTSDQADFPDTLFNGFETPDKAEPPFYVGSNGDDSFSSSGDLSLSASPVPASLAQPPLPVLPPFPPPSGKNPVMILNELRPGLKYDFLSESGESHAKSFVMSVVVDGQFFEGSGRNKKLAKARAAQSALAAIFNLHLDQTPSRQPIPSEGLQLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEEPADRHPNRKARGQLRTKIESGEGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLTP

SEQ ID NO: 7 - protein, 739 aa, Homo sapiens
MASVLGSGRGSGGLSSQLKCKSKRRRRRRSKRKDKVSILSTFLAPFKHLSPGITNTEDDDTLSTSSAEVKENRNVGNLAARPPPSGDRARGGAPGAKRKRPLEEGNGGHLCKLQLVWKKLSWSVAPKNALVQLHELRPGLQYRTVSQTGPVHAPVFAVAVEVNGLTFEGTGPTKKKAKMRAAELALRSFVQFPNACQAHLAMGGGPGPGTDFTSDQADFPDTLFQEFEPPAPRPGLAGGRPGDAALLSAAYGRRRLLCRALDLVGPTPATPAAPGERNPVVLLNRLRAGLRYVCLAEPAERRARSFVMAVSVDGRTFEGSGRSKKLARGQAAQAALQELFDIQMPGHAPGRARRTPMPQEFADSISQLVTQKFREVTTDLTPMHARHKALAGIVMTKGLDARQAQVVALSSGTKCISGEHLSDQGLVVNDCHAEVVARRAFLHFLYTQLELHLSKRREDSERSIFVRLKEGGYRLRENILFHLYVSTSPCGDARLHSPYEITTDLHSSKHLVRKFRGHLRTKIESGEGTVPVRGPSAVQTWDGVLLGEQLITMSCTDKIARWNVLGLQGALLSHFVEPVYLQSIVVGSLHHTGHLARVMSHRMEGVGQLPASYRHNRPLLSGVSDAEARQPGKSPPFSMNWVVGSADLEIINATTGRRSCGGPSRLCKHVLSARWARLYGRLSTRTPSPGDTPSMYCEAKLGAHTYQSVKQQLFKAFQKAGLGTWVRKPPEQQQFLLTL

SEQ ID NO: 8 - protein, 236 aa, Homo sapiens
MTSEKGPSTGDPTLRRRIEPWEFDVFYDPRELRKEACLLYEIKWGMSRKIWRSSGKNTTNHVEVNFIKKFTSERDFHPSMSCSITWFLSWSPCWECSQAIREFLSRHPGVTLVIYVARLFWHMDQQNRQGLRDLVNSGVTIQIMRASEYYHCWRNFVNYPPGDEAHWPQYPPLWMMLYALELHCIILSLPPCLKISRRWQNHLTFFRLHLQNCHYQTIPPHILLATGLIHPSVAWR

SEQ ID NO: 9 - protein, 224 aa, Homo sapiens
MAQKEEAAVATEAASQNGEDLENLDDPEKLKELIELPPFEIVTGERLPANFFKFQFRNVEYSSGRNKTFLCYVVEAQGKGGQVQASRGYLEDEHAAAHAEEAFFNTILPAFDPALRYNVTWYVSSSPCAACADRIIKTLSKTKNLRLLILVGRLFMWEEPEIQAALKKLKEAGCKLRIMKPQDFEYVWQNFVEQEEGESKAFQPWEDIQENFLYYEEKLADILK

SEQ ID NO: 10 - protein, 199 aa, Homo sapiens
MEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFKHCWDTFVDHQGCPFQPWDGLDEHSQALSGRLRAILQNQGN

SEQ ID NO: 11 - protein, 382 aa, Homo sapiens
MNPQIRNPMERMYRDTFYDNFENEPILYGRSYTWLCYEVKIKRGRSNLLWDTGVFRGQVYFKPQYHAEMCFLSWFCGNQLPAYKCFQITWFVSWTPCPDCVAKLAEFLSEHPNVTLTISAARLYYYWERDYRRALCRLSQAGARVTIMDYEEFAYCWENFVYNEGQQFMPWYKFDENYAFLHRTLKEILRYLMDPDTFTFNFNNDPLVLRRRQTYLCYEVERLDNGTWVLMDQHMGFLCNEAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFEYCWDTFVYRQGCPFQPWDGLEEHSQALSGRLRAILQNQGN

SEQ ID NO: 12 - protein, 190 aa, Homo sapiens
MNPQIRNPMKAMYPGTFYFQFKNLWEANDRNETWLCFTVEGIKRRSVVSWKTGVFRNQVDSETHCHAERCFLSWFCDDILSPNTKYQVTWYTSWSPCPDCAGEVAEFLARHSNVNLTIFTARLYYFQYPCYQEGLRSLSQEGVAVEIMDYEDFKYCWENFVYNDNEPFKPWKGLKTNFRLLKRRLRESLQ

SEQ ID NO: 13 - protein, 373 aa, Homo sapiens
MKPHFRNTVERMYRDTFSYNFYNRPILSRRNTVWLCYEVKTKGPSRPRLDAKIFRGQVYSQPEHHAEMCFLSWFCGNQLPAYKCFQITWFVSWTPCPDCVAKLAEFLAEHPNVTLTISAARLYYYWERDYRRALCRLSQAGARVKIMDDEEFAYCWENFVYSEGQPFMPWYKFDDNYAFLHRTLKEILRNPMEAMYPHIFYFHFKNLRKAYGRNESWLCFTMEVVKHHSPVSWKRGVFRNQVDPETHCHAERCFLSWFCDDILSPNTNYEVTWYTSWSPCPECAGEVAEFLARHSNVNLTIFTARLYYFWDTDYQEGLRSLSQEGASVEIMGYKDFKYCWENFVYNDDEPFKPWKGLKYNFLFLDSKLQEILE

SEQ ID NO: 14 - protein, 384 aa, Homo sapiens
MKPHFRNTVERMYRDTFSYNFYNRPILSRRNTVWLCYEVKTKGPSRPPLDAKIFRGQVYSELKYHPEMRFFHWFSKWRKLHRDQEYEVTWYISWSPCTKCTRDMATFLAEDPKVTLTIFVARLYYFWDPDYQEALRSLCQKRDGPRATMKIMNYDEFQHCWSKFVYSQRELFEPWNNLPKYYILLHIMLGEILRHSMDPPTFTFNFNNEPWVRGRHETYLCYEVERMHNDTWVLLNQRRGFLCNQAPHKHGFLEGRHAELCFLDVIPFWKLDLDQDYRVTCFTSWSPCFSCAQEMAKFISKNKHVSLCIFTARIYDDQGRCQEGLRTLAEAGAKISIMTYSEFKHCWDTFVDHQGCPFQPWDGLDEHSQDLSGRLRAILQNQEN

SEQ ID NO: 15 - protein, 200 aa, Homo sapiens
MALLTAETFRLQFNNKRRLRRPYYPRKALLCYQLTPQNGSTPTRGYFENKKKCHAEICFINEIKSMGLDETQCYQVTCYLTWSPCSSCAWELVDFIKAHDHLNLRIFASRLYYHWCKPQQDGLRLLCGSQVPVEVMGFPEFADCWENFVDHEKPLSFNPYKMLEELDKNSRAIKRRLDRIKIPGVRAQGRYMDILCDAEV

SEQ ID NO: 16 - protein, 367 aa, Homo sapiens
MEPIYEEYLANHGTIVKPYYWLSFSLDCSNCPYHIRTGEEARVSLTEFCQIFGFPYGTTFPQTKHLTFYELKTSSGSLVQKGHASSCTGNYIHPESMLFEMNGYLDSAIYNNDSIRHIILYSNNSPCNEANHCCISKMYNFLITYPGITLSIYFSQLYHTEMDFPASAWNREALRSLASLWPRVVLSPISGGIWHSVLHSFISGVSGSHVFQPILTGRALADRHNAYEINAITGVKPYFTDVLLQTKRNPNTKAQEALESYPLNNAFPGQFFQMPSGQLQPNLPPDLRAPVVFVLVPLRDLPPMHMGQNPNKPRNIVRHLNMPQMSFQETKDLGRLPTGRSVEIVEITEQFASSKEADEKKKKKGKK

SEQ ID NO: 17 - protein, 580 aa, Homo sapiens
MSDTWSSIQAHKKQLDSLRERLQRRRKQDSGHLDLRNPEAALSPTFRSDSPVPTAPTSGGPKPSTASAVPELATDPELEKKLLHHLSDLALTLPTDAVSICLAISTPDAPATQDGVESLLQKFAAQELIEVKRGLLQDDAHPTLVTYADHSKLSAMMGAVAEKKGPGEVAGTVTGQKRRAEQDSTTVAAFASSLVSGLNSSASEPAKEPAKKSRKHAASDVDLEIESLLNQQSTKEQQSKKVSQEILELLNTTTAKEQSIVEKFRSRGRAQVQEFCDYGTKEECMKASDADRPCRKLHFRRIINKHTDESLGDCSFLNTCFHMDTCKYVHYEIDACMDSEAPGSKDHTPSQELALTQSVGGDSSADRLFPPQWICCDIRYLDVSILGKFAVVMADPPWDIHMELPYGTLTDDEMRRLNIPVLQDDGFLFLWVTGRAMELGRECLNLWGYERVDEIIWVKTNQLQRIIRTGRTGHWLNHGKEHCLVGVKGNPQGFNQGLDCDVIVAEVRSTSHKPDEIYGMIERLSPGTRKIELFGRPHNVQPNWITLGNQLDGIHLLDPDVVARFKQRYPDGIISKPKNL

SEQ ID NO: 18 - protein, 472 aa, Homo sapiens
MSVVHQLSAGWLLDHLSFINKINYQLHQHHEPCCRKKEFTTSVHFESLQMDSVSSSGVCAAFIASDSSTKPENDDGGNYEMFTRKFVFRPELFDVTKPYITPAVHKECQQSNEKEDLMNGVKKEISISIIGKKRKRCVVFNQGELDAMEYHTKIRELILDGSLQLIQEGLKSGFLYPLFEKQDKGSKPITLPLDACSLSELCEMAKHLPSLNEMEHQTLQLVEEDTSVTEQDLFLRVVENNSSFTKVITLMGQKYLLPPKSSFLLSDISCMQPLLNYRKTFDVIVIDPPWQNKSVKRSNRYSYLSPLQIQQIPIPKLAAPNCLLVTWVTNRQKHLRFIKEELYPSWSVEVVAEWHWVKITNSGEFVFPLDSPHKKPYEGLILGRVQEKTALPLRNADVNVLPIPDHKLIVSVPCTLHSHKPPLAEVLKDYIKPDGEYLELFARNLQPGWTSWGNEVLKFQHVDYFIAVESGS

SEQ ID NO: 19 - protein, 419 aa, Homo sapiens
MSVVHQLSAGWLLDHLSFINKINYQLHQHHEPCCRKKEFTTSVHFESLQMDSVSSSGVCAAFIASDSSTKPENDDGGNYEMFTRKFVFRPELFDVTKPYITPAVHKECQQSNEKEDLMNGVKKEISISIIGKKRKRCVVFNQGELDAMEYHTKIRELILDGSLQLIQEGLKSGFLYPLFEKQDKGSKPITLPLDACSLSELCEMAKHLPSLNEMEHQTLQLVEEDTSVTEQDLFLRVVENNSSFTKVITLMGQKYLLPPKSSFLLSDISCMQPLLNYRKTFDVIVIDPPWQNKSVKRSNRYSYLSPLQIQQIPIPKLAAPNCLLVTWVTNRQKHLRFIKEELYPSWSVEVVAEWHWVKITNSGEFVFPLDSPHKKPYEGLILGRVQEKTALPLRGFKRLHQARWGIFGVVCSKFTARLD

SEQ ID NO: 20 - protein, 456 aa, Homo sapiens
MDSRLQEIRERQKLRRQLLAQQLGAESADSIGAVLNSKDEQREIAETRETCRASYDTSAPNAKRKYLDEGETDEDKMEEYKDELEMQQDEENLPYEEEIYKDSSTFLKGTQSLNPHNDYCQHFVDTGHRPQNFIRDVGLADRFEEYPKLRELIRLKDELIAKSNTPPMYLQADIEAFDIRELTPKFDVILLEPPLEEYYRETGITANEKCWTWDDIMKLEIDEIAAPRSFIFLWCGSGEGLDLGRVCLRKWGYRRCEDICWIKTNKNNPGKTKTLDPKAVFQRTKEHCLMGIKGTVKRSTDGDFIHANVDIDLIITEEPEIGNIEKPVEIFHIIEHFCLGRRRLHLFGRDSTIRPGWLTVGPTLTNSNYNAETYASYFSAPNSYLTGCTEEIERLRPKSPPPKSKSDRGGGAPRGGGRGGTSAGRGRERNRSNFRGERGGFRGGRGGAHRGGFPPR

SEQ ID NO: 21 - protein, 396 aa, Homo sapiens
MTNEEPLPKKVRLSETDFKVMARDELILRWKQYEAYVQALEGKYTDLNSNDVTGLRESEEKLKQQQQESARRENILVMRLATKEQEMQECTTQIQYLKQVQQPSVAQLRSTMVDPAINLFFLKMKGELEQTKDKLEQAQNELSAWKFTPDSQTGKKLMAKCRMLIQENQELGRQLSQGRIAQLEAELALQKKYSEELKSSQDELNDFIIQLDEEVEGMQSTILVLQQQLKETRQQLAQYQQQQSQASAPSTSRTTASEPVEQSEATSKDCSRLTNGPSNGSSSRQRTSGSGFHREGNTTEDDFPSSPGNGNKSSNSSEERTGRGGSGYVNQLSAGYESVDSPTGSENSLTHQSNDTDSSHDPQEEKAVSGKGNRTVGSRHVQNGLDSSVNVQGSVL

SEQ ID NO: 22 - protein, 151 aa, Homo sapiens
MTNEEPLPKKVRLSETDFKVMARDELILRWKQYEAYVQALEGKYTDLNSNDVTGLRESEEKLKQQQQESARRENILVMRLATKEQEMQECTTQIQYLKQVQQPSVAQLRSTMVDPAINLFFLKMKGELEQTKDKLEQAQNELSAWKFTPDR

SEQ ID NO: 23 - protein, 170 aa, Homo sapiens
MTNEEPLPKKVRLSETDFKVMARDELILRWKQYEAYVQALEGKYTDLNSNDVTGLRESEEKLKQQQQESARRENILVMRLATKEQEMQECTTQIQYLKQVQQPSVAQLRSTMVDPAINLFFLKMKGELEQTKDKLEQAQNELSAWKFTPDRGLMASDYSEEVATSEKFPF

SEQ ID NO: 24 - protein, 394 aa, Homo sapiens
MAAASGYTDLREKLKSMTSRDNYKAGSREAAAAAAAAVAAAAAAAAAAEPYPVSGAKRKYQEDSDPERSDYEEQQLQKEEEARKVKSGIRQMRLFSQDECAKIEARIDEVVSRAEKGLYNEHTVDRAPLRNKYFFGEGYTYGAQLQKRGPGQERLYPPGDVDEIPEWVHQLVIQKLVEHRVIPEGFVNSAVINDYQPGGCIVSHVDPIHIFERPIVSVSFFSDSALCFGCKFQFKPIRVSEPVLSLPVRRGSVTVLSGYAADEITHCIRPQDIKERRAVIILRKTRLDAPRLETKSLSSSVLPPSYASDRLSGNNRDPALKPKRSHRKADPDAAHRPRILEMDKEENRRSVLLPTHRRRGSFSSENYWRKSYESSEDCSEAAGSPARKVKMRRH

SEQ ID NO: 25 - protein, 70 aa, Artificial Sequence
  Description of Artificial Sequence: Synthetic polypeptide
  SITE (1)..(70): This sequence may encompass 1-10 repeating “(Gly)n Ser” units, wherein n = 1-10
  SITE (1)..(6): This region may encompass 2-6 residues
  SITE (8)..(13): This region may encompass 2-6 residues
  SITE (15)..(20): This region may encompass 2-6 residues
  SITE (22)..(27): This region may encompass 2-6 residues
  SITE (29)..(34): This region may encompass 2-6 residues
  SITE (36)..(41): This region may encompass 2-6 residues
  SITE (43)..(48): This region may encompass 2-6 residues
  SITE (50)..(55): This region may encompass 2-6 residues
  SITE (57)..(62): This region may encompass 2-6 residues
  SITE (64)..(69): This region may encompass 2-6 residues
GGGGGGSGGGGGGSGGGGGGSGGGGGGSGGGGGGSGGGGGGSGGGGGGSGGGGGGSGGGGGGSGGGGGGS

SEQ ID NO: 26-70 - skipped (45 entries)

SEQ ID NO: 71 - protein, 11 aa, Artificial Sequence
  Description of Artificial Sequence: Synthetic peptide
LQKKLEELELA

SEQ ID NO: 72 - skipped

SEQ ID NO: 73 - protein, 11 aa, Artificial Sequence
  Description of Artificial Sequence: Synthetic peptide
ELALKLAGLDI

SEQ ID NO: 74 - protein, 11 aa, Artificial Sequence
  Description of Artificial Sequence: Synthetic peptide
LQLPPLERLTL

SEQ ID NO: 75 - protein, 6 aa, Artificial Sequence
  Description of Artificial Sequence: Synthetic 6xHis tag
HHHHHH

SEQ ID NO: 76 - protein, 18 aa, Artificial Sequence
  Description of Artificial Sequence: Synthetic peptide
GGGGSLQLPPLERLTLGS

SEQ ID NO: 77 - protein, 16 aa, Artificial Sequence
  Description of Artificial Sequence: Synthetic peptide
GGSGGGSSGAAAGSGG

SEQ ID NO: 78 - protein, 12 aa, Artificial Sequence
  Description of Artificial Sequence: Synthetic peptide
SLQLPPLERLTL

SEQ ID NO: 79 - protein, 11 aa, Artificial Sequence
  Description of Artificial Sequence: Synthetic peptide
  MOD_RES (2)..(3): Any naturally occurring amino acid
  MOD_RES (6)..(7): Any naturally occurring amino acid
  MOD_RES (9)..(9): Any naturally occurring amino acid
  MOD_RES (11)..(11): Any naturally occurring amino acid
LXXKLXXLXLX

SEQ ID NO: 80 - protein, 15 aa, Artificial Sequence
  Description of Artificial Sequence: Synthetic peptide
CIQQQLGQLTLENTL

SEQ ID NO: 81 - protein, 12 aa, Artificial Sequence
  Description of Artificial Sequence: Synthetic peptide
ALQKKLEELELD

SEQ ID NO: 82 - protein, 12 aa, Artificial Sequence
  Description of Artificial Sequence: Synthetic peptide
TLWQFLLHLLLD

SEQ ID NO: 83 - DNA, 42 bp, Artificial Sequence
  Description of Artificial Sequence: Synthetic primer
ctgatctagaggtaccggatcagtgtcgccctcgaacttcac

SEQ ID NO: 84 - DNA, 21 bp, Artificial Sequence
  Description of Artificial Sequence: Synthetic oligonucleotide
gtgggccagggcacgggcagc

SEQ ID NO: 85 - DNA, 21 bp, Artificial Sequence
  Description of Artificial Sequence: Synthetic oligonucleotide
gggtgggccagggcacgggca

SEQ ID NO: 86 - DNA, 21 bp, Artificial Sequence
  Description of Artificial Sequence: Synthetic oligonucleotide
gagggtgggccagggcacggg

SEQ ID NO: 87 - DNA, 21 bp, Artificial Sequence
  Description of Artificial Sequence: Synthetic oligonucleotide
acgagggtgggccagggcacg

SEQ ID NO: 88 - DNA, 21 bp, Artificial Sequence
  Description of Artificial Sequence: Synthetic oligonucleotide
tcacgagggtgggccagggca

SEQ ID NO: 89 - DNA, 21 bp, Artificial Sequence
  Description of Artificial Sequence: Synthetic oligonucleotide
ggtcacgagggtgggccaggg

SEQ ID NO: 90 - DNA, 21 bp, Artificial Sequence
  Description of Artificial Sequence: Synthetic oligonucleotide
gtggtcacgagggtgggccag

SEQ ID NO: 91 - DNA, 25 bp, Artificial Sequence
  Description of Artificial Sequence: Synthetic oligonucleotide
gggtggtcacgagggtgggccaggg

SEQ ID NO: 92 - DNA, 25 bp, Artificial Sequence
  Description of Artificial Sequence: Synthetic oligonucleotide
agggtggtcacgagggtgggccagg

SEQ ID NO: 93 - DNA, 25 bp, Artificial Sequence
  Description of Artificial Sequence: Synthetic oligonucleotide
cagggtggtcacgagggtgggccag

SEQ ID NO: 94 - DNA, 27 bp, Artificial Sequence
  Description of Artificial Sequence: Synthetic oligonucleotide
  Description of Combined DNA/RNA Molecule: Synthetic oligonucleotide
ucagggtggtcacgagggtgggccagg

SEQ ID NO: 95 - DNA, 27 bp, Artificial Sequence
  Description of Artificial Sequence: Synthetic oligonucleotide
  Description of Combined DNA/RNA Molecule: Synthetic oligonucleotide
gucagggtggtcacgagggtgggccag

SEQ ID NO: 96 - RNA, 29 nt, Artificial Sequence
  Description of Artificial Sequence: Synthetic oligonucleotide
cccuggcccacccucgugaccacccugac

SEQ ID NO: 97 - RNA, 31 nt, Artificial Sequence
  Description of Artificial Sequence: Synthetic oligonucleotide
gucaggguggucacgagggugggccagggca

SEQ ID NO: 98 - DNA, 42 bp, Homo sapiens
tgtacccaaaaagcagccaggaatatgtcataccagggcttt

SEQ ID NO: 99 - RNA, 29 nt, Artificial Sequence
  Description of Artificial Sequence: Synthetic oligonucleotide
aaaaggcagccaggaauaucucauaccaa

SEQ ID NO: 100 - RNA, 31 nt, Artificial Sequence
  Description of Artificial Sequence: Synthetic oligonucleotide
uugguaugagauauuccuggcugccuuuugg

SEQ ID NO: 101 - DNA, 40 bp, Homo sapiens
atagtttcaagtagctatgatggtctctgtcgcatctggg

SEQ ID NO: 102 - RNA, 29 nt, Artificial Sequence
  Description of Artificial Sequence: Synthetic oligonucleotide
aaguggcuaugauggucuccgucgcauca

SEQ ID NO: 103 - RNA, 31 nt, Artificial Sequence
  Description of Artificial Sequence: Synthetic oligonucleotide
ugaugcgacggagaccaucauagccacuuga

SEQ ID NO: 104 - RNA, 53 nt, Homo sapiens
ucggauccagucucggccguucauuuuaaucgugauggauccuugauaguuuc

SEQ ID NO: 105 - RNA, 23 nt, Artificial Sequence
  Description of Artificial Sequence: Synthetic oligonucleotide
ucaaggauccgaacggccgagac

SEQ ID NO: 106 - DNA, 3816 bp, Artificial Sequence
  Description of Artificial Sequence: Synthetic polynucleotide
atggactacaaagacgatgacgacaaggcggccgcacagctgcatttaccgcaggttttagctgacgctgtctcacgcctggtcctgggtaagtttggtgacctgaccgacaacttctcctcccctcacgctcgcagaaaagtgctggctggagtcgtcatgacaacaggcacagatgttaaagatgccaaggtgataagtgtttctacaggaacaaaatgtattaatggtgaatacatgagtgatcgtggccttgcattaaatgactgccatgcagaaataatatctcggagatccttgctcagatttctttatacacaacttgagctttacttaaataacaaagatgatcaaaaaagatccatctttcagaaatcagagcgaggggggtttaggctgaaggagaatgtccagtttcatctgtacatcagcacctctccctgtggagatgccagaatcttctcaccacatgagccaatcctggaagaaccagcagatagacacccaaatcgtaaagcaagaggacagctacggaccaaaatagagtctggtcaggggacgattccagtgcgctccaatgcgagcatccaaacgtgggacggggtgctgcaaggggagcggctgctcaccatgtcctgcagtgacaagattgcacgctggaacgtggtgggcatccagggatcactgctcagcattttcgtggagcccatttacttctcgagcatcatcctgggcagcctttaccacggggaccacctttccagggccatgtaccagcggatctccaacatagaggacctgccacctctctacaccctcaacaagcctttgctcagtggcatcagcaatgcagaagcacggcagccagggaaggcccccaacttcagtgtcaactggacggtaggcgactccgctattgaggtcatcaacgccacgactgggaaggatgagctgggccgcgcgtcccgcctgtgtaagcacgcgttgtactgtcgctggatgcgtgtgcacggcaaggttccctcccacttactacgctccaagattaccaagcccaacgtgtaccatgagtccaagctggcggcaaaggagtaccaggccgccaaggcgcgtctgttcacagccttcatcaaggcggggctgggggcctgggtggagaagcccaccgagcaggaccagttctcactcacgggaggtggcggtagtctgcagctgcctccacttgaaagactgacactgggatccgaagcgggaccctcgggagcagctgcgggcgcttacctgccccccctgcagcaggtgttccaggcacctcgccggcctggcattggcactgtggggaaaccaatcaagctcctggccaattactttgaggtggacatccctaagatcgacgtgtaccactacgaggtggacatcaagccggataagtgtccccgtagagtcaaccgggaagtggtggaatacatggtccagcatttcaagcctcagatctttggtgatcgcaagcctgtgtatgatggaaagaagaacatttacactgtcacagcactgcccattggcaacgaacgggtcgactttgaggtgacaatccctggggaagggaaggatcgaatctttaaggtctccatcaagtggctagccattgtgagctggcgaatgctgcatgaggccctggtcagcggccagatccctgttcccttggagtctgtgcaagccctggatgtggccatgaggcacctggcatccatgaggtacacccctgtgggccgctccttcttctcaccgcctgagggctactaccacccgctggggggtgggcgcgaggtctggttcggctttcaccagtctgtgcgccctgccatgtggaagatgatgctcaacattgatgtctcagccactgccttttataaggcacagccagtgattgagttcatgtgtgaggtgctggacatcaggaacatagatgagcagcccaagcccctcacggactctcagcgcgttcgcttcaccaaggagatcaagggcctgaaggtggaagtcacccactgtggacagatgaagaggaagtaccgcgtgtgtaatgttacccgtcgccctgctagccatcagacattccccttacagctggagagtggacagactgtggagtgcacagtggcacagtatttcaagcagaaatataaccttcagctcaagtatccccatctgccctgcctacaagttggccaggaacaaaagcatacctaccttcccctagaggtctgtaacattgtggctgggcagcgctgtattaaaaagctgaccgacaaccagacctcgaccatgataaaggccacagctagatccgctccagacagacaggaggagatcagtcgcctgatgaagaatgccagctacaacttagatccctacatccaggaatttgggatcaaagtgaaggatgacatgacggaggtgacagggcgagtgctgccggcgcccatcttgcagtacggcggccggaaccgggccattgccacacccaatcagggtgtctgggacatgcgggggaaacagttctacaatgggattgagatcaaagtctgggccatcgcctgcttcgcaccccaaaaacagtgtcgagaagaggtgctcaagaacttcacagaccagctgcggaagatttccaaggatgcggggatgcctatccagggtcaaccttgtttctgcaaatatgcacagggggcagacagcgtggagcctatgttccggcatctcaagaacacctactcagggctgcagctcattattgtcatcctgccagggaagacgccggtgtatgctgaggtgaaacgtgtcggagatacactcttgggaatggctacgcagtgtgtgcaggtgaagaacgtggtcaagacctcacctcagactctgtccaacctctgcctcaagatcaatgtcaaacttggtggcattaacaacatcctagtcccacaccagcgctctgccgtttttcaacagccagtgatattcctgggagcagatgttacacaccccccagcaggggatgggaaaaaaccttctatcacagcagtggtaggcagtatggatgcccaccccagccgatactgtgctactgtgcgggtacagcgaccacggcaagagatcattgaagacttgtcctacatggtgcgtgagctcctcatccaattctacaagtccacccgtttcaagcctacccgcatcatcttctaccgagatggggtgcctgaaggccagctaccccagatactccactatgagctactggccattcgtgatgcctgcatcaaactggaaaaggactaccagcctgggatcacttatattgtggtgcagaaacgccatcacacccgccttttctgtgctgacaagaatgagcgaattgggaagagtggtaacatcccagctgggaccacagtggacaccaacatcacccacccatttgagtttgacttctatctgtgcagccacgcaggcatccagggcaccagccgaccatcccattactatgttctttgggatgacaaccgtttcacagcagatgagctccagatcctgacgtaccagctgtgccacacttacgtacgatgcacacgctctgtctctatcccagcacctgcctactatgcccgcctggtggctttccgggcacgataccacctggtggacaaggagcatgacagtggagaggggagccacatatcggggcagagcaatgggcgggacccccaggccctggccaaagccgtgcaggttcaccaggatactctgcgcaccatgtacttcgcttga

SEQ ID NO: 107 - DNA, 3822 bp, Artificial Sequence
  Description of Artificial Sequence: Synthetic polynucleotide
atggactacaaagacgatgacgacaaggcggccgcacagctgcatttaccgcaggttttagctgacgctgtctcacgcctggtcctgggtaagtttggtgacctgaccgacaacttctcctcccctcacgctcgcagaaaagtgctggctggagtcgtcatgacaacaggcacagatgttaaagatgccaaggtgataagtgtttctacaggaggcaaatgtattaatggtgaatacatgagtgatcgtggccttgcattaaatgactgccatgcagaaataatatctcggagatccttgctcagatttctttatacacaacttgagctttacttaaataacaaagatgatcaaaaaagatccatctttcagaaatcagagcgaggggggtttaggctgaaggagaatgtccagtttcatctgtacatcagcacctctccctgtggagatgccagaatcttctcaccacatgagccaatcctggaagaaccagcagatagacacccaaatcgtaaagcaagaggacagctacggaccaaaatagagtctggtcaggggacgattccagtgcgctccaatgcgagcatccaaacgtgggacggggtgctgcaaggggagcggctgctcaccatgtcctgcagtgacaagattgcacgctggaacgtggtgggcatccagggatcactgctcagcattttcgtggagcccatttacttctcgagcatcatcctgggcagcctttaccacggggaccacctttccagggccatgtaccagcggatctccaacatagaggacctgccacctctctacaccctcaacaagcctttgctcagtggcatcagcaatgcagaagcacggcagccagggaaggcccccaacttcagtgtcaactggacggtaggcgactccgctattgaggtcatcaacgccacgactgggaaggatgagctgggccgcgcgtcccgcctgtgtaagcacgcgttgtactgtcgctggatgcgtgtgcacggcaaggttccctcccacttactacgctccaagattaccaagcccaacgtgtaccatgagtccaagctggcggcaaaggagtaccaggccgccaaggcgcgtctgttcacagccttcatcaaggcggggctgggggcctgggtggagaagcccaccgagcaggaccagttctcactcacgggaggtggcggtagtctgcagctgcctccacttgaaagactgacactgggatcctactcgggagccggccccgcacttgcacctcctgcgccgccgccccccatccaaggatatgccttcaagcctccacctagacccgactttgggacctccgggagaacaatcaaattacaggccaatttcttcgaaatggacatccccaaaattgacatctatcattatgaattggatatcaagccagagaagtgcccgaggagagttaacagggaaatcgtggaacacatggtccagcactttaaaacacagatctttggggatcggaagcccgtgtttgacggcaggaagaatctatacacagccatgccccttccgattgggagggacaaggtggagctggaggtcacgctgccaggagaaggcaaggatcgcatcttcaaggtgtccatcaagtgggtgtcctgcgtgagcttgcaggcgttacacgatgcactttcagggcggctgcccagcgtcccttttgagacgatccaggccctggacgtggtcatgaggcacttgccatccatgaggtacacccccgtgggccgctccttcttcaccgcgtccgaaggctgctctaaccctcttggcgggggccgagaagtgtggtttggcttccatcagtccgtccggccttctctctggaaaatgatgctgaatattgatgtgtcagcaacagcgttttacaaggcacagccagtaatcgagtttgtttgtgaagttttggattttaaaagtattgaagaacaacaaaaacctctgacagattcccaaagggtaaagtttaccaaagaaattaaaggtctaaaggtggagataacgcactgtgggcagatgaagaggaagtaccgcgtctgcaatgtgacccggcggcccgccagtcaccaaacattcccgctgcagcaggagagcgggcagacggtggagtgcacggtggcccagtatttcaaggacaggcacaagttggttctgcgctacccccacctcccatgtttacaagtcggacaggagcagaaacacacctaccttcccctggaggtctgtaacattgtggcaggacaaagatgtattaaaaaattaacggacaatcagacctcaaccatgatcagagcgactgctaggtcggcgcccgatcggcaagaagagattagcaaattgatgcgaagtgcaagtttcaacacagatccatacgtccgtgaatttggaatcatggtcaaagatgagatgacagacgtgactgggcgggtgctgcagccgccctccatcctctacgggggcaggaataaagctattgcgacccctgtccagggcgtctgggacatgcggaacaagcagttccacacgggcatcgagatcaaggtgtgggccattgcgtgcttcgccccccagcgccagtgcacggaagtccatctgaagtccttcacagagcagctcagaaagatctcgagagacgccggcatgcccatccagggccagccgtgcttctgcaaatacgcgcagggggcggacagcgtggagcccatgttccggcacctgaagaacacgtatgcgggcctgcagctggtggtggtcatcctgcccggcaagacgcccgtgtacgccgaggtcaagcgcgtgggagacacggtgctggggatggccacgcagtgcgtgcagatgaagaacgtgcagaggaccacgccacagaccctgtccaacctctgcctgaagatcaacgtcaagctgggaggcgtgaacaacatcctgctgccccagggcaggccgccggtgttccagcagcccgtcatctttctgggagcagccgtcactcacccccccgccggggatgggaagaagccctccattgccgccgtggtgggcagcatggacgcccaccccaatcgctactgcgccaccgtgcgcgtgcagcagcaccggcaggagatcatacaagacctggccgccatggtccgcgagctcctcatccagttctacaagtccacgcgcttcaagcccacccgcatcatcttctaccgcgccggtgtctctgaaggccagttccagcaggttctccaccacgagttgctggccatccgtgaggcctgtatcaagctagaaaaagactaccagcccgggatcaccttcatcgtggtgcagaagaggcaccacacccggctcttctgcactgacaagaacgagcgggttgggaaaagtggaaacattccagcaggcacgactgtggacacgaaaatcacccaccccaccgagttcgacttctacctgtgtagtcacgctggcatccaggggacaagcaggccttcgcactatcacgtcctctgggacgacaatcgtttctcctctgatgagctgcagatcctaacctaccagctgtgtcacacctacgtgcgctgcacacgctccgtgtccatcccagcgccagcatactacgctcacctggtggccttccgggccaggtaccacctggtggataaggaacatgacagtgctgaaggaagccatacctctgggcagagtaacgggcgagaccaccaagcactggccaaggcggtccaggttcaccaagacactctgcgcaccatgtactttgcttga

SEQ ID NO: 108 - DNA, 3672 bp, Artificial Sequence
  Description of Artificial Sequence: Synthetic polynucleotide
atggactacaaagacgatgacgacaaggcggccgcacagctgcatttaccgcaggttttagctgacgctgtctcacgcctggtcctgggtaagtttggtgacctgaccgacaacttctcctcccctcacgctcgcagaaaagtgctggctggagtcgtcatgacaacaggcacagatgttaaagatgccaaggtgataagtgtttctacaggaggcaaatgtattaatggtgaatacatgagtgatcgtggccttgcattaaatgactgccatgcagaaataatatctcggagatccttgctcagatttctttatacacaacttgagctttacttaaataacaaagatgatcaaaaaagatccatctttcagaaatcagagcgaggggggtttaggctgaaggagaatgtccagtttcatctgtacatcagcacctctccctgtggagatgccagaatcttctcaccacatgagccaatcctggaagaaccagcagatagacacccaaatcgtaaagcaagaggacagctacggaccaaaatagagtctggtcaggggacgattccagtgcgctccaatgcgagcatccaaacgtgggacggggtgctgcaaggggagcggctgctcaccatgtcctgcagtgacaagattgcacgctggaacgtggtgggcatccagggatcactgctcagcattttcgtggagcccatttacttctcgagcatcatcctgggcagcctttaccacggggaccacctttccagggccatgtaccagcggatctccaacatagaggacctgccacctctctacaccctcaacaagcctttgctcagtggcatcagcaatgcagaagcacggcagccagggaaggcccccaacttcagtgtcaactggacggtaggcgactccgctattgaggtcatcaacgccacgactgggaaggatgagctgggccgcgcgtcccgcctgtgtaagcacgcgttgtactgtcgctggatgcgtgtgcacggcaaggttccctcccacttactacgctccaagattaccaagcccaacgtgtaccatgagtccaagctggcggcaaaggagtaccaggccgccaaggcgcgtctgttcacagccttcatcaaggcggggctgggggcctgggtggagaagcccaccgagcaggaccagttctcactcacgggaggtggcggtagtctgcagctgcctccacttgaaagactgacactgggatccattgacatctatcattatgaattggatatcaagccagagaagtgcccgaggagagttaacagggaaatcgtggaacacatggtccagcactttaaaacacagatctttggggatcggaagcccgtgtttgacggcaggaagaatctatacacagccatgccccttccgattgggagggacaaggtggagctggaggtcacgctgccaggagaaggcaaggatcgcatcttcaaggtgtccatcaagtgggtgtcctgcgtgagcttgcaggcgttacacgatgcactttcagggcggctgcccagcgtcccttttgagacgatccaggccctggacgtggtcatgaggcacttgccatccatgaggtacacccccgtgggccgctccttcttcaccgcgtccgaaggctgctctaaccctcttggcgggggccgagaagtgtggtttggcttccatcagtccgtccggccttctctctggaaaatgatgctgaatattgatgtgtcagcaacagcgttttacaaggcacagccagtaatcgagtttgtttgtgaagttttggattttaaaagtattgaagaacaacaaaaacctctgacagattcccaaagggtaaagtttaccaaagaaattaaaggtctaaaggtggagataacgcactgtgggcagatgaagaggaagtaccgcgtctgcaatgtgacccggcggcccgccagtcaccaaacattcccgctgcagcaggagagcgggcagacggtggagtgcacggtggcccagtatttcaaggacaggcacaagttggttctgcgctacccccacctcccatgtttacaagtcggacaggagcagaaacacacctaccttcccctggaggtctgtaacattgtggcaggacaaagatgtattaaaaaattaacggacaatcagacctcaaccatgatcagagcgactgctaggtcggcgcccgatcggcaagaagagattagcaaattgatgcgaagtgcaagtttcaacacagatccatacgtccgtgaatttggaatcatggtcaaagatgagatgacagacgtgactgggcgggtgctgcagccgccctccatcctctacgggggcaggaataaagctattgcgacccctgtccagggcgtctgggacatgcggaacaagcagttccacacgggcatcgagatcaaggtgtgggccattgcgtgcttcgccccccagcgccagtgcacggaagtccatctgaagtccttcacagagcagctcagaaagatctcgagagacgccggcatgcccatccagggccagccgtgcttctgcaaatacgcgcagggggcggacagcgtggagcccatgttccggcacctgaagaacacgtatgcgggcctgcagctggtggtggtcatcctgcccggcaagacgcccgtgtacgccgaggtcaagcgcgtgggagacacggtgctggggatggccacgcagtgcgtgcagatgaagaacgtgcagaggaccacgccacagaccctgtccaacctctgcctgaagatcaacgtcaagctgggaggcgtgaacaacatcctgctgccccagggcaggccgccggtgttccagcagcccgtcatctttctgggagcagccgtcactcacccccccgccggggatgggaagaagccctccattgccgccgtggtgggcagcatggacgcccaccccaatcgctactgcgccaccgtgcgcgtgcagcagcaccggcaggagatcatacaagacctggccgccatggtccgcgagctcctcatccagttctacaagtccacgcgcttcaagcccacccgcatcatcttctaccgcgccggtgtctctgaaggccagttccagcaggttctccaccacgagttgctggccatccgtgaggcctgtatcaagctagaaaaagactaccagcccgggatcaccttcatcgtggtgcagaagaggcaccacacccggctcttctgcactgacaagaacgagcgggttgggaaaagtggaaacattccagcaggcacgactgtggacacgaaaatcacccaccccaccgagttcgacttctacctgtgtagtcacgctggcatccaggggacaagcaggccttcgcactatcacgtcctctgggacgacaatcgtttctcctctgatgagctgcagatcctaacctaccagctgtgtcacacctacgtgcgctgcacacgctccgtgtccatcccagcgccagcatactacgctcacctggtggccttccgggccaggtaccacctggtggataaggaacatgacagtgctgaaggaagccatacctctgggcagagtaacgggcgagaccaccaagcactggccaaggcggtccaggttcaccaagacactctgcgcaccatgtactttgcttga

SEQ ID NO: 109 - DNA, 3825 bp, Artificial Sequence
  Description of Artificial Sequence: Synthetic polynucleotide
atggactacaaagacgatgacgacaaggcggccgcacagctgcatttaccgcaggttttagctgacgctgtctcacgcctggtcctgggtaagtttggtgacctgaccgacaacttctcctcccctcacgctcgcagaaaagtgctggctggagtcgtcatgacaacaggcacagatgttaaagatgccaaggtgataagtgtttctacaggaacaaaatgtattaatggtgaatacatgagtgatcgtggccttgcattaaatgactgccatgcagaaataatatctcggagatccttgctcagatttctttatacacaacttgagctttacttaaataacaaagatgatcaaaaaagatccatctttcagaaatcagagcgaggggggtttaggctgaaggagaatgtccagtttcatctgtacatcagcacctctccctgtggagatgccagaatcttctcaccacatgagccaatcctggaagaaccagcagatagacacccaaatcgtaaagcaagaggacagctacggaccaaaatagagtctggtcaggggacgattccagtgcgctccaatgcgagcatccaaacgtgggacggggtgctgcaaggggagcggctgctcaccatgtcctgcagtgacaagattgcacgctggaacgtggtgggcatccagggatcactgctcagcattttcgtggagcccatttacttctcgagcatcatcctgggcagcctttaccacggggaccacctttccagggccatgtaccagcggatctccaacatagaggacctgccacctctctacaccctcaacaagcctttgctcagtggcatcagcaatgcagaagcacggcagccagggaaggcccccaacttcagtgtcaactggacggtaggcgactccgctattgaggtcatcaacgccacgactgggaaggatgagctgggccgcgcgtcccgcctgtgtaagcacgcgttgtactgtcgctggatgcgtgtgcacggcaaggttccctcccacttactacgctccaagattaccaagcccaacgtgtaccatgagtccaagctggcggcaaaggagtaccaggccgccaaggcgcgtctgttcacagccttcatcaaggcggggctgggggcctgggtggagaagcccaccgagcaggaccagttctcactcacgggaggtggcggtagtctgcagctgcctccacttgaaagactgacactgggatccgaaatcggctccgcaggacccgctggggcccagcccctactcatggtgcccagaagacctggctatggcaccatgggcaaacccattaaactgctggctaactgttttcaagttgaaatcccaaagattgatgtctacctctatgaggtagatattaaaccagacaagtgtcctaggagagtgaacagggaggtggttgactcaatggttcagcattttaaagtaactatatttggagaccgtagaccagtttatgatggaaaaagaagtctttacaccgccaatccacttcctgtggcaactacaggggtagatttagacgttactttacctggggaaggtggaaaagatcgacctttcaaggtgtcaatcaaatttgtctctcgggtgagttggcacctactgcatgaagtactgacaggacggaccttgcctgagccactggaattagacaagccaatcagcactaaccctgtccatgccgttgatgtggtgctacgacatctgccctccatgaaatacacacctgtggggcgttcatttttctccgctccagaaggatatgaccaccctctgggagggggcagggaagtgtggtttggattccatcagtctgttcggcctgccatgtggaaaatgatgcttaatatcgatgtttctgccactgccttctacaaagcacaacctgtaattcagttcatgtgtgaagttcttgatattcataatattgatgagcaaccaagacctctgactgattctcatcgggtaaaattcaccaaagagataaaaggtttgaaggttgaagtgactcattgtggaacaatgagacggaaataccgtgtttgtaatgtaacaaggaggcctgccagtcatcaaacctttcctttacagttagaaaacggccaaactgtggagagaacagtagcgcagtatttcagagaaaagtatactcttcagctgaagtacccgcaccttccctgtctgcaagtcgggcaggaacagaaacacacctacctgccactagaagtctgtaatattgtggcagggcaacgatgtatcaagaagctaacagacaatcagacttccactatgatcaaggcaacagcaagatctgcaccagatagacaagaggaaattagcagattggtaagaagtgcaaattatgaaacagatccatttgttcaggagtttcaatttaaagttcgggatgaaatggctcatgtaactggacgcgtacttccagcacctatgctccagtatggaggacggaatcggacagtagcaacaccgagccatggagtatgggacatgcgagggaaacaattccacacaggagttgaaatcaaaatgtgggctatcgcttgttttgccacacagaggcagtgcagagaagaaatattgaagggtttcacagaccagctgcgtaagatttctaaggatgcagggatgcccatccagggccagccatgcttctgcaaatatgcacagggggcagacagcgtagagcccatgttccggcatctcaagaacacatattctggcctacagcttattatcgtcatcctgccggggaagacaccagtgtatgcggaagtgaaacgtgtaggagacacacttttgggtatggctacacaatgtgttcaagtcaagaatgtaataaaaacatctcctcaaactctgtcaaacttgtgcctaaagataaatgttaaactcggagggatcaataatattcttgtacctcatcaaagaccttctgtgttccagcaaccagtgatctttttgggagccgatgtcactcatccacctgctggtgatggaaagaagccttctattgctgctgttgtaggtagtatggatgcacacccaagcagatactgtgccacagtaagagttcagagaccccgacaggagatcatccaggacttggcctccatggtccgggaacttcttattcaattttataagtcaactcggttcaagcctactcgtatcatcttttatcgggatggtgtttcagaggggcagtttaggcaggtattatattatgaactactagcaattcgagaagcctgcatcagtttggagaaagactatcaacctggaataacctacattgtagttcagaagagacatcacactcgattattttgtgctgataggacagaaagggttggaagaagtggcaatatcccagctggaacaacagttgatacagacattacacacccatatgagttcgatttttacctctgtagccatgctggaatacagggtaccagtcgtccttcacactatcatgttttatgggatgataactgctttactgcagatgaacttcagctgctaacttaccagctctgccacacttacgtacgctgtacacgatctgtttctatacctgcaccagcgtattatgctcacctggtagcatttagagccagatatcatcttgtggacaaagaacatgacagtgctgaaggaagtcacgtttcaggacaaagcaatgggcgagatccacaagctcttgccaaggctgtacagattcaccaagataccttacgcacaatgtacttcgcttaa

SEQ ID NO: 110 - DNA, 3828 bp, Artificial Sequence
  Description of Artificial Sequence: Synthetic polynucleotide
atggactacaaagacgatgacgacaaggcggccgcacagctgcatttaccgcaggttttagctgacgctgtctcacgcctggtcctgggtaagtttggtgacctgaccgacaacttctcctcccctcacgctcgcagaaaagtgctggctggagtcgtcatgacaacaggcacagatgttaaagatgccaaggtgataagtgtttctacaggaacaaaatgtattaatggtgaatacatgagtgatcgtggccttgcattaaatgactgccatgcagaaataatatctcggagatccttgctcagatttctttatacacaacttgagctttacttaaataacaaagatgatcaaaaaagatccatctttcagaaatcagagcgaggggggtttaggctgaaggagaatgtccagtttcatctgtacatcagcacctctccctgtggagatgccagaatcttctcaccacatgagccaatcctggaagaaccagcagatagacacccaaatcgtaaagcaagaggacagctacggaccaaaatagagtctggtcaggggacgattccagtgcgctccaatgcgagcatccaaacgtgggacggggtgctgcaaggggagcggctgctcaccatgtcctgcagtgacaagattgcacgctggaacgtggtgggcatccagggatcactgctcagcattttcgtggagcccatttacttctcgagcatcatcctgggcagcctttaccacggggaccacctttccagggccatgtaccagcggatctccaacatagaggacctgccacctctctacaccctcaacaagcctttgctcagtggcatcagcaatgcagaagcacggcagccagggaaggcccccaacttcagtgtcaactggacggtaggcgactccgctattgaggtcatcaacgccacgactgggaaggatgagctgggccgcgcgtcccgcctgtgtaagcacgcgttgtactgtcgctggatgcgtgtgcacggcaaggttccctcccacttactacgctccaagattaccaagcccaacgtgtaccatgagtccaagctggcggcaaaggagtaccaggccgccaaggcgcgtctgttcacagccttcatcaaggcggggctgggggcctgggtggagaagcccaccgagcaggaccagttctcactcacgggaggtggcggtagtctgcagctgcctccacttgaaagactgacactgggatccgaggcgctgggacccggacctccggctagcctgtttcagccacctcgtcgtcctggccttggaactgttggaaaaccaattcgactgttagccaatcattttcaggttcagattcctaaaatagatgtgtatcactatgatgtggatattaagcctgaaaaacggcctcgtagagtcaacagggaggtagtagatacaatggtgcggcacttcaagatgcaaatatttggtgatcggcagcctgggtatgatggcaaaagaaacatgtacacagcacatccactaccaattggacgggatagggttgatatggaggtgactcttccaggcgagggtaaagaccaaacatttaaagtgtctgttcagtgggtgtcagttgtgagccttcagttgcttttagaagctttggctgggcacttgaatgaagtcccagatgactcagtacaagcacttgatgttatcacaagacaccttccctccatgaggtacaccccagtgggccgttcctttttctcacccccggaaggttactaccaccctctgggagggggcagggaggtctggtttggttttcatcagtctgtgagacctgccatgtggaatatgatgctcaacattgatgtatctgcaactgctttctaccgggctcagcctatcattgagttcatgtgtgaggttttagacattcagaacatcaatgaacagaccaaacctctaacagactcccagcgtgtcaaatttaccaaagaaatcagaggtctcaaagttgaggtgacccactgtggacagatgaaacgaaaataccgagtttgtaatgtgactagacggccagccagtcatcaaacttttcctttgcagctagaaaacggtcaagctatggaatgtacagtagctcaatattttaagcaaaagtatagtctgcaactgaaatacccccatcttccctgtctccaagtgggacaagaacaaaagcatacatacttgccactcgaggtctgtaatatagtggcaggacagcgatgtatcaagaagctcacagacaatcagacttccacaatgatcaaagctacagcaagatctgctcctgacagacaggaagagatcagtagactggtgaagagcaacagtatggtgggtggacctgatccataccttaaagaatttggtattgttgtccacaatgaaatgacagagctcacaggcagggtacttccagcaccaatgctgcaatatggaggccggaataaaacagtagccacacccaaccagggtgtctgggacatgcgaggaaagcagttttatgctggcattgaaattaaagtttgggcagttgcttgttttgcacctcagaaacaatgtagggaagatttactaaagagtttcactgaccagctgcgtaaaatctctaaggatgcaggaatgcccatccagggtcagccatgtttctgcaagtatgcacaaggtgcagacagtgtggagcctatgtttaaacatctgaaaatgacttatgtgggcctacagctaatagtggttatcctgcctggaaagacaccagtatatgcggaggtgaaacgtgttggagatacccttctaggtatggccacacagtgtgtccaggtaaaaaatgtagtgaagacctcacctcaaaccctttccaatctttgcctgaagataaatgcaaaacttggaggaattaacaatgtgcttgtgcctcatcaaaggccctcggtgttccagcagcctgtcatcttcctgggagcggatgtcacacaccccccagcaggggatgggaagaaaccttccattgctgctgtggttggcagtatggatggccaccccagccggtactgtgccaccgttcgggtgcagacttcccggcaggagatctcccaagagctcctctacagtcaagaggtcatccaggacctgactaacatggttcgagagctgctgattcagttctacaaatccacacgcttcaaacccactcggatcatctattaccgtggaggggtatctgagggacaaatgaaacaggtagcttggccagaactaatagcaattcgaaaggcatgtattagcttggaagaagattaccggccaggaataacttatattgtggtgcaaaaaagacatcacacacgactcttctgtgcagataaaacagaaagggtagggaaaagtggcaatgtaccagcaggcactacagtggatagtaccatcacacatccatctgagtttgacttttacctctgtagtcatgcaggaattcagggaaccagccgtccctcacattaccaggtcttgtgggatgacaactgcttcactgcagatgaactccagctactgacttaccagctgtgtcacacctatgtgaggtgcactcgctcagtctctattccagcccctgcatattatgcccggcttgtagcatttagggcaaggtatcatctggtggataaagatcatgacagtgcggaaggcagtcatgtgtcaggacagagcaacggccgggatcctcaggccttggctaaggctgtgcaaatccaccatgatacccagcacacgatgtattttgcctga

SEQ ID NO: 111 - DNA, 3261 bp, Artificial Sequence
  Description of Artificial Sequence: Synthetic polynucleotide
atggactacaaagacgatgacgacaaggcggccgcagaagccagcccagcatccgggcccagacacttgatggatccacacatattcacttccaactttaacaatggcattggaaggcataagacctacctgtgctacgaagtggagcgcctggacaatggcacctcggtcaagatggaccagcacaggggctttctacacaaccaggctaagaatcttctctgtggcttttacggccgccatgcggagctgcgcttcttggacctggttccttctttgcagttggacccggcccagatctacagggtcacttggttcatctcctggagcccctgcttctcctggggctgtgccggggaagtgcgtgcgttccttcaggagaacacacacgtgagactgcgtatcttcgctgcccgcatctatgatgacgaccccctatataaggaggcactgcaaatgctgcgggatgctggggcccaagtctccatcatgacctacgatgaatttaagcactgctgggacacctttgtggaccaccagggatgtcccttccagccctgggatggactagatgagcacagccaagccctgagtgggaggctgcgggccattctccagaatcagggaaacggaggtggcggtagtctgcagctgcctccacttgaaagactgacactgggatcctactcgggagccggccccgcacttgcacctcctgcgccgccgccccccatccaaggatatgccttcaagcctccacctagacccgactttgggacctccgggagaacaatcaaattacaggccaatttcttcgaaatggacatccccaaaattgacatctatcattatgaattggatatcaagccagagaagtgcccgaggagagttaacagggaaatcgtggaacacatggtccagcactttaaaacacagatctttggggatcggaagcccgtgtttgacggcaggaagaatctatacacagccatgccccttccgattgggagggacaaggtggagctggaggtcacgctgccaggagaaggcaaggatcgcatcttcaaggtgtccatcaagtgggtgtcctgcgtgagcttgcaggcgttacacgatgcactttcagggcggctgcccagcgtcccttttgagacgatccaggccctggacgtggtcatgaggcacttgccatccatgaggtacacccccgtgggccgctccttcttcaccgcgtccgaaggctgctctaaccctcttggcgggggccgagaagtgtggtttggcttccatcagtccgtccggccttctctctggaaaatgatgctgaatattgatgtgtcagcaacagcgttttacaaggcacagccagtaatcgagtttgtttgtgaagttttggattttaaaagtattgaagaacaacaaaaacctctgacagattcccaaagggtaaagtttaccaaagaaattaaaggtctaaaggtggagataacgcactgtgggcagatgaagaggaagtaccgcgtctgcaatgtgacccggcggcccgccagtcaccaaacattcccgctgcagcaggagagcgggcagacggtggagtgcacggtggcccagtatttcaaggacaggcacaagttggttctgcgctacccccacctcccatgtttacaagtcggacaggagcagaaacacacctaccttcccctggaggtctgtaacattgtggcaggacaaagatgtattaaaaaattaacggacaatcagacctcaaccatgatcagagcgactgctaggtcggcgcccgatcggcaagaagagattagcaaattgatgcgaagtgcaagtttcaacacagatccatacgtccgtgaatttggaatcatggtcaaagatgagatgacagacgtgactgggcgggtgctgcagccgccctccatcctctacgggggcaggaataaagctattgcgacccctgtccagggcgtctgggacatgcggaacaagcagttccacacgggcatcgagatcaaggtgtgggccattgcgtgcttcgccccccagcgccagtgcacggaagtccatctgaagtccttcacagagcagctcagaaagatctcgagagacgccggcatgcccatccagggccagccgtgcttctgcaaatacgcgcagggggcggacagcgtggagcccatgttccggcacctgaagaacacgtatgcgggcctgcagctggtggtggtcatcctgcccggcaagacgcccgtgtacgccgaggtcaagcgcgtgggagacacggtgctggggatggccacgcagtgcgtgcagatgaagaacgtgcagaggaccacgccacagaccctgtccaacctctgcctgaagatcaacgtcaagctgggaggcgtgaacaacatcctgctgccccagggcaggccgccggtgttccagcagcccgtcatctttctgggagcagccgtcactcacccccccgccggggatgggaagaagccctccattgccgccgtggtgggcagcatggacgcccaccccaatcgctactgcgccaccgtgcgcgtgcagcagcaccggcaggagatcatacaagacctggccgccatggtccgcgagctcctcatccagttctacaagtccacgcgcttcaagcccacccgcatcatcttctaccgcgccggtgtctctgaaggccagttccagcaggttctccaccacgagttgctggccatccgtgaggcctgtatcaagctagaaaaagactaccagcccgggatcaccttcatcgtggtgcagaagaggcaccacacccggctcttctgcactgacaagaacgagcgggttgggaaaagtggaaacattccagcaggcacgactgtggacacgaaaatcacccaccccaccgagttcgacttctacctgtgtagtcacgctggcatccaggggacaagcaggccttcgcactatcacgtcctctgggacgacaatcgtttctcctctgatgagctgcagatcctaacctaccagctgtgtcacacctacgtgcgctgcacacgctccgtgtccatcccagcgccagcatactacgctcacctggtggccttccgggccaggtaccacctggtggataaggaacatgacagtgctgaaggaagccatacctctgggcagagtaacgggcgagaccaccaagcactggccaaggcggtccaggttcaccaagacactctgcgcaccatgtactttgcttga

SEQ ID NO: 112 - DNA, 3261 bp, Artificial Sequence
  Description of Artificial Sequence: Synthetic polynucleotide
atggactacaaagacgatgacgacaaggcggccgcagaagccagcccagcatccgggcccagacacttgatggatccacacatattcacttccaactttaacaatggcattggaaggcataagacctacctgtgctacgaagtggagcgcctggacaatggcacctcggtcaagatggaccagcacaggggctttctacacaaccaggctaagaatcttctctgtggcttttacggccgccatgcggagctgcgcttcttggacctggttccttctttgcagttggacccggcccagatctacagggtcacttggttcatctcctggagcccctgcttctcctggggctgtgccggggaagtgcgtgcgttccttcaggagaacacacacgtgagactgcgtatcttcgctgcccgcatctatgatcgcgaccccctatataaggaggcactgcaaatgctgcgggatgctggggcccaagtctccatcatgacctacgatgaatttaagcactgctgggacacctttgtggaccaccagggatgtcccttccagccctgggatggactagatgagcacagccaagccctgagtgggaggctgcgggccattctccagaatcagggaaacggaggtggcggtagtctgcagctgcctccacttgaaagactgacactgggatcctactcgggagccggccccgcacttgcacctcctgcgccgccgccccccatccaaggatatgccttcaagcctccacctagacccgactttgggacctccgggagaacaatcaaattacaggccaatttcttcgaaatggacatccccaaaattgacatctatcattatgaattggatatcaagccagagaagtgcccgaggagagttaacagggaaatcgtggaacacatggtccagcactttaaaacacagatctttggggatcggaagcccgtgtttgacggcaggaagaatctatacacagccatgccccttccgattgggagggacaaggtggagctggaggtcacgctgccaggagaaggcaaggatcgcatcttcaaggtgtccatcaagtgggtgtcctgcgtgagcttgcaggcgttacacgatgcactttcagggcggctgcccagcgtcccttttgagacgatccaggccctggacgtggtcatgaggcacttgccatccatgaggtacacccccgtgggccgctccttcttcaccgcgtccgaaggctgctctaaccctcttggcgggggccgagaagtgtggtttggcttccatcagtccgtccggccttctctctggaaaatgatgctgaatattgatgtgtcagcaacagcgttttacaaggcacagccagtaatcgagtttgtttgtgaagttttggattttaaaagtattgaagaacaacaaaaacctctgacagattcccaaagggtaaagtttaccaaagaaattaaaggtctaaaggtggagataacgcactgtgggcagatgaagaggaagtaccgcgtctgcaatgtgacccggcggcccgccagtcaccaaacattcccgctgcagcaggagagcgggcagacggtggagtgcacggtggcccagtatttcaaggacaggcacaagttggttctgcgctacccccacctcccatgtttacaagtcggacaggagcagaaacacacctaccttcccctggaggtctgtaacattgtggcaggacaaagatgtattaaaaaattaacggacaatcagacctcaaccatgatcagagcgactgctaggtcggcgcccgatcggcaagaagagattagcaaattgatgcgaagtgcaagtttcaacacagatccatacgtccgtgaatttggaatcatggtcaaagatgagatgacagacgtgactgggcgggtgctgcagccgccctccatcctctacgggggcaggaataaagctattgcgacccctgtccagggcgtctgggacatgcggaacaagcagttccacacgggcatcgagatcaaggtgtgggccattgcgtgcttcgccccccagcgccagtgcacggaagtccatctgaagtccttcacagagcagctcagaaagatctcgagagacgccggcatgcccatccagggccagccgtgcttctgcaaatacgcgcagggggcggacagcgtggagcccatgttccggcacctgaagaacacgtatgcgggcctgcagctggtggtggtcatcctgcccggcaagacgcccgtgtacgccgaggtcaagcgcgtgggagacacggtgctggggatggccacgcagtgcgtgcagatgaagaacgtgcagaggaccacgccacagaccctgtccaacctctgcctgaagatcaacgtcaagctgggaggcgtgaacaacatcctgctgccccagggcaggccgccggtgttccagcagcccgtcatctttctgggagcagccgtcactcacccccccgccggggatgggaagaagccctccattgccgccgtggtgggcagcatggacgcccaccccaatcgctactgcgccaccgtgcgcgtgcagcagcaccggcaggagatcatacaagacctggccgccatggtccgcgagctcctcatccagttctacaagtccacgcgcttcaagcccacccgcatcatcttctaccgcgccggtgtctctgaaggccagttccagcaggttctccaccacgagttgctggccatccgtgaggcctgtatcaagctagaaaaagactaccagcccgggatcaccttcatcgtggtgcagaagaggcaccacacccggctcttctgcactgacaagaacgagcgggttgggaaaagtggaaacattccagcaggcacgactgtggacacgaaaatcacccaccccaccgagttcgacttctacctgtgtagtcacgctggcatccaggggacaagcaggccttcgcactatcacgtcctctgggacgacaatcgtttctcctctgatgagctgcagatcctaacctaccagctgtgtcacacctacgtgcgctgcacacgctccgtgtccatcccagcgccagcatactacgctcacctggtggccttccgggccaggtaccacctggtggataaggaacatgacagtgctgaaggaagccatacctctgggcagagtaacgggcgagaccaccaagcactggccaaggcggtccaggttcaccaagacactctgcgcaccatgtactttgcttga

SEQ ID NO: 113 - protein, 15 aa, Artificial Sequence
  Description of Artificial Sequence: Synthetic peptide
GSLQLPPLERLTLGS

SEQ ID NO: 114 - protein, 202 aa, Homo sapiens
MNPQIRNPMERMYRDTFYDNFENEPILYGRSYTWLCYEVKIKRGRSNLLWDTGVFRGPVLPKRQSNHRQEVDPETHCHAERCFLSWFCDDILSPNTNYEVTWYTSWSPCPECAGEVAEFLARHSNVNLTIFTARLCYFWDTDYQEGLCSLSQEGASVKIMGYKDFVSCWKNFVYSDDEPFKPWKGLQTNFRLLKRRLREILQ

SEQ ID NO: 115 - protein, 8 aa, Artificial Sequence
  Description of Artificial Sequence: Synthetic peptide
DYKDDDDK

SEQ ID NO: 116 - protein, 9 aa, Artificial Sequence
  Description of Artificial Sequence: Synthetic peptide
YPYDVPDYA

SEQ ID NO: 117 - protein, 10 aa, Artificial Sequence
  Description of Artificial Sequence: Synthetic peptide
EQKLISEEDL

SEQ ID NO: 118 - protein, 6 aa, Artificial Sequence
  Description of Artificial Sequence: Synthetic peptide
DTYRYI

SEQ ID NO: 119 - DNA, 36 bp, Artificial Sequence
  Description of Artificial Sequence: Synthetic oligonucleotide
aagctgcccgtgccctggcccaccctcgtgaccacc

SEQ ID NO: 120 - DNA, 36 bp, Artificial Sequence
  Description of Artificial Sequence: Synthetic oligonucleotide
aagctgcccgtgccctagcccaccctcgtgaccacc

SEQ ID NO: 121 - DNA, 36 bp, Artificial Sequence
  Description of Artificial Sequence: Synthetic oligonucleotide
  modified_base (17)..(17): inosine
aagctgcccgtgccctngcccaccctcgtgaccacc

SEQ ID NO: 122 - DNA, 42 bp, Artificial Sequence
  Description of Artificial Sequence: Synthetic oligonucleotide
aagctgcccgtgccctagcccaccctcgtgaccaccctgacc

SEQ ID NO: 123 - RNA, 31 nt, Artificial Sequence; identical to SEQ ID NO: 97
  Description of Artificial Sequence: Synthetic oligonucleotide

SEQ ID NO: 124 - DNA, 29 bp, Artificial Sequence
  Description of Artificial Sequence: Synthetic oligonucleotide
  Description of Combined DNA/RNA Molecule: Synthetic oligonucleotide
ccctggcccacccucgugaccacccugac

SEQ ID NO: 125 - DNA, 20 bp, Artificial Sequence
  Description of Artificial Sequence: Synthetic oligonucleotide
gggtgggctagggcacgggc

SEQ ID NO: 126 - DNA, 19 bp, Artificial Sequence
  Description of Artificial Sequence: Synthetic oligonucleotide
gggtgggctagggcacggg

SEQ ID NO: 127 - DNA, 37 bp, Artificial Sequence
  Description of Artificial Sequence: Synthetic oligonucleotide
gtctcggccgttcattttaatcgtgatggatccttga

SEQ ID NO: 128 - DNA, 21 bp, Artificial Sequence
  Description of Artificial Sequence: Synthetic oligonucleotide
  Description of Combined DNA/RNA Molecule: Synthetic oligonucleotide
gtggucacgagggugggccag

SEQ ID NO: 129 - RNA, 21 nt, Artificial Sequence
  Description of Artificial Sequence: Synthetic oligonucleotide
ggucacgagggugggccaggg

SEQ ID NO: 130 - RNA, 22 nt, Artificial Sequence
  Description of Artificial Sequence: Synthetic oligonucleotide
ucacgaggguugggccagggca

SEQ ID NO: 131 - RNA, 21 nt, Artificial Sequence
  Description of Artificial Sequence: Synthetic oligonucleotide
acgagggugggccagggcacg

SEQ ID NO: 132 - RNA, 21 nt, Artificial Sequence
  Description of Artificial Sequence: Synthetic oligonucleotide
gagggugggccagggcacggg

SEQ ID NO: 133 - RNA, 21 nt, Artificial Sequence
  Description of Artificial Sequence: Synthetic oligonucleotide
gggugggccagggcacgggca

SEQ ID NO: 134 - DNA, 22 bp, Artificial Sequence
  Description of Artificial Sequence: Synthetic oligonucleotide
  Description of Combined DNA/RNA Molecule: Synthetic oligonucleotide
gutgggccagggcacgggcagc

SEQ ID NO: 135 - RNA, 24 nt, Artificial Sequence
  Description of Artificial Sequence: Synthetic oligonucleotide
ggguggucacgagggugggcaggg

SEQ ID NO: 136 - RNA, 24 nt, Artificial Sequence
  Description of Artificial Sequence: Synthetic oligonucleotide
aggguggucacgagggugggcagg

SEQ ID NO: 137 - RNA, 24 nt, Artificial Sequence
  Description of Artificial Sequence: Synthetic oligonucleotide
caggguggucacgagggugggcag

SEQ ID NO: 138 - RNA, 26 nt, Artificial Sequence
  Description of Artificial Sequence: Synthetic oligonucleotide
ucaggguggucacgagggugggcagg

SEQ ID NO: 139 - RNA, 26 nt, Artificial Sequence
  Description of Artificial Sequence: Synthetic oligonucleotide
gucaggguggucacgagggugggcag

SEQ ID NO: 140 - DNA, 37 bp, Artificial Sequence
  Description of Artificial Sequence: Synthetic oligonucleotide
agtctcggccgttcattttaatcgtgatggatccttg

SEQ ID NO: 141 - RNA, 31 nt, Artificial Sequence; identical to SEQ ID NO: 100
  Description of Artificial Sequence: Synthetic oligonucleotide

SEQ ID NO: 142 - DNA, 34 bp, Artificial Sequence
  Description of Artificial Sequence: Synthetic oligonucleotide
tctcggccgttcattttaatcgtgatggatcctt

SEQ ID NO: 143 - DNA, 24 bp, Artificial Sequence
  Description of Artificial Sequence: Synthetic oligonucleotide
gacatattcctggctgctttttgg

SEQ ID NO: 144 - RNA, 23 nt, Artificial Sequence
  Description of Artificial Sequence: Synthetic oligonucleotide
ucaaggauccgaacggccgagac

SEQ ID NO: 145 - RNA, 31 nt, Artificial Sequence; identical to SEQ ID NO: 103
  Description of Artificial Sequence: Synthetic oligonucleotide

SEQ ID NO: 146 - DNA, 29 bp, Artificial Sequence
  Description of Artificial Sequence: Synthetic oligonucleotide
aagtggctatgatggtctccgtcgcatca

SEQ ID NO: 147 - DNA, 24 bp, Artificial Sequence
  Description of Artificial Sequence: Synthetic oligonucleotide
gtttcaagtagctatgatggtctc

(19) United States
(12) Patent Application Publication
(10) Pub. No.: US 2024/0209339 A1
(43) Pub. Date: Jun. 27, 2024

(54) REDIRECTING RISC FOR RNA EDITING

(71) Applicant: ALNYLAM PHARMACEUTICALS, INC., Cambridge, MA (US)

(72) Inventors: Min TAN, Cambridge, MA (US); Christopher BROWN, Cambridge, MA (US); Vasant JADHAV, Cambridge, MA (US)

(73) Assignee: ALNYLAM PHARMACEUTICALS, INC., Cambridge, MA (US)

(21) Appl. No.: 18/556,180

(22) PCT Filed: Apr. 29, 2022
(86) PCT No.: PCT/US2022/026984

§ 371 Date: Oct. 19, 2023

Related U.S. Application Data
(60) Provisional application No. 63/182,241, filed on Apr. 30, 2021.

(51) Int. Cl. C12N 9/78; C12N 15/113

(52) U.S. Cl. CPC C12N 9/78; C12N 15/113; C07K 2319/095; C07K 2319/43; C12N 2310/11; C12N 2310/533; C12Y 305/04004; C12Y 305/04005

(57) ABSTRACT

The disclosure provides systems, compositions, kits, and methods useful for the targeted site-specific modifications of RNA molecules. Generally, the systems, compositions, kits, and methods described herein comprise a polypeptide or a nucleic acid encoding the polypeptide. The polypeptide comprises a first domain comprising a catalytic domain of an RNA modifying enzyme and a second domain comprising a MID domain of an Argonaute (Ago) protein. The systems, compositions, kits, and methods can also comprise an oligonucleotide for targeting the polypeptide to a target RNA. A method for modifying a target RNA, comprises contacting the target RNA with a polypeptide or a nucleic acid encoding the polypeptide and with an oligonucleotide described herein. Some exemplary modifications of the target RNA include, but are not limited to, site-specific deamination of an adenosine, deamination of a cytidine, methylation (e.g., methylation at position 6) of an adenosine, and demethylation of m6-adenosine in the target RNA.

### CROSS-REFERENCE TO RELATED APPLICATIONS

This application a 35 U.S.C. § 371 National Phase Entry Application of International Patent Application No. PCT/US2022/026984 filed on Apr. 30, 2022, which designated the U.S., and which claims benefit under 35 U.S.C. § 119(e) of the U.S. Provisional Application No. 63/182,241, filed Apr. 30, 2021, the contents of both of which are incorporated herein by reference in their entireties.

### SEQUENCE LISTING

The instant application contains a Sequence Listing which has been submitted electronically in ASCII format and is hereby incorporated by reference in its entirety. Said ASCII copy, created on Jun. 13, 2022, is named 051058-099570WOPT_SL.txt and is 157,659 bytes in size.

### TECHNICAL FIELD

The technology described herein relates to systems, compositions, kits, and methods that are useful for the targeted editing of RNA molecules.

### BACKGROUND

Targeted modification of nucleic acid sequences, for example, the targeted introduction of a specific modification into RNA, is a highly promising approach for the study of gene function that has the potential to provide new therapies for genetic diseases. Thus, there is a need in the art for methods and compositions for targeted modification of RNAs. The present disclosure addresses some of these needs.

### SUMMARY

Provided herein are systems, compositions, kits, and methods for modifying a target RNA. Generally, the systems, compositions, kits, and methods described herein comprise a polypeptide or a nucleic acid encoding the polypeptide. The polypeptide comprises a first domain comprising a catalytic domain of an RNA modifying enzyme and a second domain comprising a MID domain of an Argonaute (Ago) protein.

The systems, compositions, kits, and methods can also comprise an oligonucleotide for targeting the polypeptide to a target RNA. The oligonucleotide can be single-stranded or double-stranded. In some embodiments, the oligonucleotide is double-stranded. For example, the oligonucleotide comprises a double-stranded (duplex) region of at least 15, 16, 17, or 18 nucleotide base-pairs.

In another aspect, provided herein is a method for modifying a target RNA. Generally, the method comprises contacting the target RNA with a polypeptide or a nucleic acid encoding the polypeptide and with an oligonucleotide described herein. Some exemplary modifications of the target RNA include, but are not limited to, site-specific deamination of an adenosine, deamination of a cytidine, methylation (e.g., methylation at position 6) of an adenosine demethylation of m⁶-adenosine in the target RNA.

It is noted that contacting with the target cell can be in a cell. Further, contacting with the target RNA can be in vitro or in vivo.

In yet another aspect, provided herein is a cell comprising a polypeptide, a nucleic acid encoding the polypeptide, or an oligonucleotide described herein.

### BRIEF DESCRIPTION OF THE DRAWINGS

This patent or application file contains at least one drawing executed in color. Copies of this patent or patent application publication with color drawing(s) will be provided by the Office upon request and payment of the necessary fee.

FIG. 1 is a schematic representation of RNA editing by Adenosine Deaminases Acting on RNA (ADAR) and exemplary ADAR polypeptides.

FIG. 2 is a schematic representation of an exemplary GFP Reporter Assay for RNA Editing. Selective editing of adenosine to inosine within the STOP codon of the W58X mutant restores translation of eGFP, turning on fluorescence with the successfully affected cells. FIG. 2 discloses SEQ ID NOS 119-121, respectively, in order of appearance.

FIG. 3 is a schematic representation of ADAR-Ago2 fusion polypeptide and exemplary GFP reporter and siRNA sequences for RNA editing according to an embodiment. FIG. 3 discloses SEQ ID NOS 122-124, respectively, in order of appearance.

FIG. 4 are photographs showing RNA Editing by ADAR-Ago2 and an editing siRNA according to an embodiment.

FIG. 5 shows sequencing results confirming ADAR-hAgo2 changes the sequence of reporter RNA. FIG. 5 discloses SEQ ID NOS 125-126, respectively, in order of appearance.

FIG. 6 is a schematic representation of ADAR-hAgo2 fusion polypeptide and exemplary GFP reporter and siRNA antisense sequences for RNA editing according to an embodiment and FIG. 6 discloses SEQ ID NOS 119-120, and 128-134, respectively, in order of appearance.

FIG. 7 is a schematic representation of ADAR-hAgo2 fusion polypeptide and exemplary GFP reporter and siRNA antisense sequences for RNA editing according to an embodiment. FIG. 7 discloses SEQ ID NOS 119, 120, and 135-139, respectively, in order of appearance.

FIG. 8 are photographs showing RNA Editing by ADAR-Ago2. Editing was seen in a small number of cells with editing siRNA7 (see FIG. 6) (C:A mismatch at antisense position 19; 19/21 design)

FIG. 9 are photographs showing RNA editing with some exemplary modifications of hAgo2. The N-terminal amino acids 1˜51 may be important for either siRNA loading or target recognition. FIG. 9 discloses SEQ ID NO: 77.

FIG. 10 is a schematic of workflow for editing endogenous targets with polypeptides described herein.

FIG. 11 shows exemplary sequencing results confirming ADAR-hAgo2 and editing siRNA changes the sequence of LeuRS (LARS1) mRNA. FIG. 11 discloses SEQ ID NOS 98, 141, 99, 143, and 143, respectively, in order of appearance.

FIG. 12 shows sequencing results confirming ADAR-hAgo2 and editing siRNA changes the sequence of WDR5 mRNA. FIG. 12 discloses SEQ ID NOS 101, 145-147, and 147, respectively, in order of appearance.

FIG. 13 are photographs showing RNA editing with exemplary polypeptide comprising different human Agos. As can be seen RNA editing was seen with ADAR-Ago1˜4.

FIG. 14 is a schematic representation of RNA editing by RNA deaminase (APOBEC family proteins and ADAR).

FIG. 15 is a schematic representation of an APOBEC3A-Ago2 fusion polypeptide and exemplary reporter and antisense strand sequences of siRNA for C to U RNA editing according to an embodiment. FIG. 15 discloses SEQ ID NOS 104 and 144, respectively, in order of appearance.

FIG. 16 shows exemplary sequencing results confirming C to U RNA editing using the exemplary APOBEC3A-Ago2 fusion polypeptide and editing siRNA of FIG. 15. FIG. 16 discloses SEQ ID NOS 140, 142, and 127, respectively, in order of appearance.

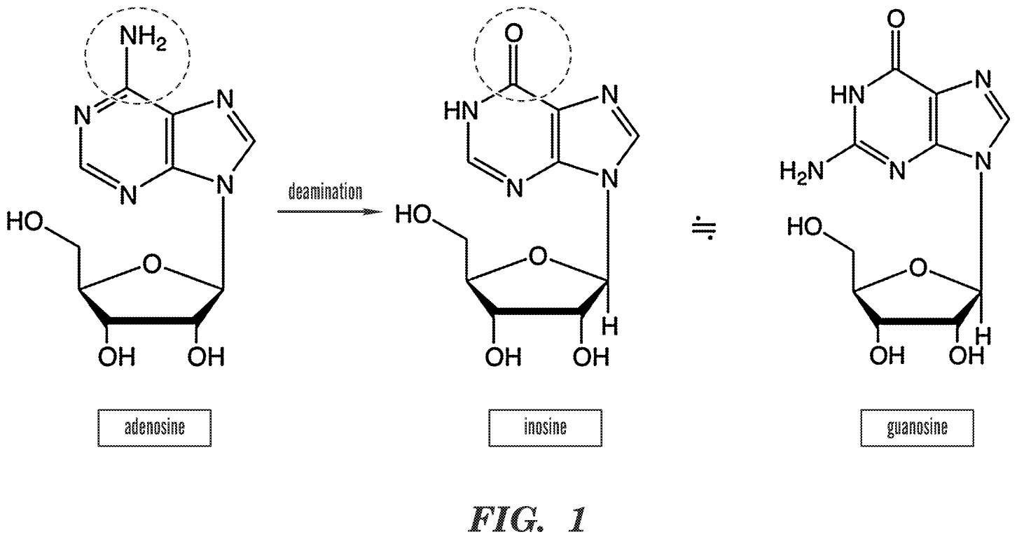

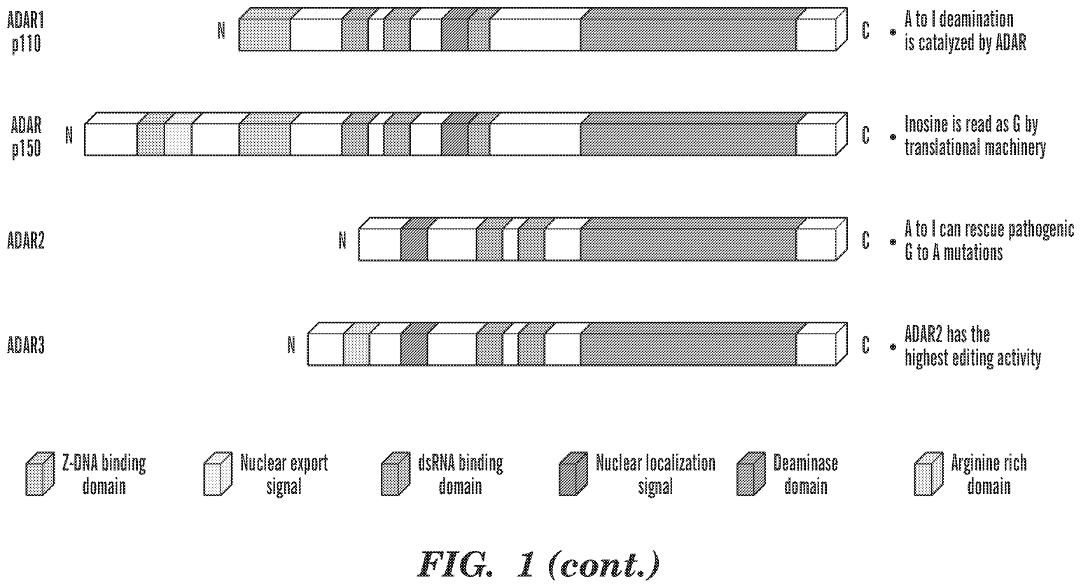

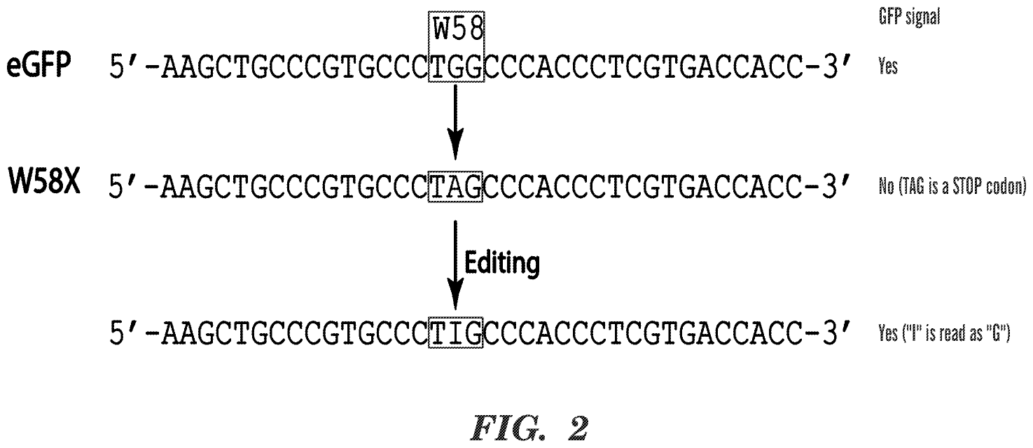

### DETAILED DESCRIPTION

Various aspects described herein include a polypeptide comprising a first domain comprising a catalytic domain of an RNA modifying enzyme and a second domain comprising a MID domain of an Ago protein. Exemplary catalytic domains and Ago protein domains are described herein below.

### Ago Domain

In some embodiments of any one of the aspects described herein, the polypeptide comprises a MID domain of an Argonaute protein. Argonaute proteins are proteins of the PIWI protein superfamily that contain an N-terminal (N), a Piwi-Argonaute-Zwille (PAZ), a middle (MID), and a P-element-induced wimpy testis (PIWI) domain. Ago are capable of binding small RNAs, such as microRNAs, small interfering RNAs (siRNAs), and Piwi-interacting RNAs. Agos can be guided to target sequences with these RNAs in order to cleave mRNA, inhibit translation, or induce mRNA degradation in the target sequence. Generally, the domains are connected in some arrangements by structured linker regions. Agos possessing this structural layout, which include prokaryotic and eukaryotic Agos, are considered “long.” However, there also exists a class of “short” Agos which only possess MID and PIWI domain. The 5′-end of the guide is sequestered in a region of the MID domain. While the residues involved in this binding are somewhat conserved, some marked differences exist between eukaryotic Agos and prokaryotic Agos. The 3′-end of the guide is bound by the PAZ domain. The catalytic region of Agos is an RNase H-like fold located in the PIWI domain, which utilized a conserved DEDX (X=D or H) tetrad for catalysis. Mutations to these residues renders the Argonaute inactive. Also included in the argonaute family of proteins as described herein are the Piwi subfamily of proteins such as Hili, Hiwi, Hiwi 2 and Hiwi3.

The mammalian Ago family comprises eight members, four of which are ubiquitously expressed (Ago subfamily), with the remaining four (Piwi subfamily) being expressed in germ cells. While Ago2 has been shown to be at the core of the RISC complex that carries out oligonucleotide-guided target RNA cleavage in the region of complementarity, Ago1, 3, and 4 are thought to lack this cleavage activity and may therefore function in related oligonucleotide-guided gene silencing pathways that do not involve target RNA cleavage in the region of complementarity. Similarly, Ago2 may function in gene silencing independent of such cleavage activity, such as in translational repression.

In some embodiments of any one of the aspects, Ago protein can be from Anoxybacillus flavithermus, Aquifex aeolicus, Aquifex aeolicus strain VF5, Arabidopsis thaliana, Archaeoglobus fulgidus, Aromatoleum aromaticum, Clostridium bartlettii, D. melanogaster, Exiguobacterium, Halogeometricum borinquense, Halorubrum lacusprofundi, Microsystis aeruginosa, Pyrococcus furiosus, Synechococcus, Synechococcus elongatus, Thermosynechococcus elogatus, Thermus thermophilus, Thermus thermophilus JL-18, or Thermus thermophilus strain HB27.

Exemplary sequences for Agos can be found in Genebank with Accession Numbers as listed: human Ago1 (NP 036331); human Ago2 (NP 036286), human Ago3 (NP 079128), human Ago4 (NP 060099)Hili (NP 060538), Hiwi (NP 0047553), Hiwi2 (NP 689644), Hiwi3 (NP 001008496), Drosophila melanogaster (Dm) Ago 1 (NP 725341), Dm Ago2 (NP 730054), Dm Ago3 (AB027430), Aubergine (CAA64320), PIWI (NP 476875), Arabidopsis thaliana (At) Ago1 (NP 849784), At Ago2 (NP 174413), At Ago3 (NP 174414), At Ago4 (NP 565633), At Ago5 (At2g27880), At Ago6 (At2g32940), At Ago7 (NP 1771033), At Ago8 (NP 1976023), At Ago9 (CAD66636), At Ago 10 (NP 199194), Schizosaccharomyces pombe (Sp) Ago (NP 587782) and Caenorhabditis elegans (Ce) Alg-1 (fNP 5103221).

It is noted that MID domain can from a eukaryotic or prokaryotic Ago. In some embodiments of any one of the aspects, the MID domain is from a mammalian Ago such as a mouse or human Ago. For example, the MID domain is from human Ago (hAgo) 1, hAgo2, hAgo3 or hAgo4. In some preferred embodiments, the MID domain is from hAgo2.

In some embodiments of any one of the aspects, the second domain of the polypeptide further comprises a PAZ domain of an Ago. It is noted that PAZ domain can from a eukaryotic or prokaryotic Ago. In some embodiments of any one of the aspects, the PAZ domain is from a mammalian Ago such as a mouse or human Ago. For example, the PAZ domain is from human Ago (hAgo) 1, hAgo2, hAgo3 or hAgo4. In some preferred embodiments, the PAZ domain is from hAgo2

The MID domain and the PAZ domain and can be from the same Ago or different Agos. For example, one of the MID domain or the PAZ domain can be from Ago1, and the other of the MID domain or the PAZ domain can be from Ago2, Ago3 or Ago4. In another example, one of the MID domain or the PAZ domain can be from Ago2, and the other of the MID domain or the PAZ domain can be from Ago3 or Ago4. In yet another non-limiting example, one of the MID domain or the PAZ domain can be from Ago3, and the other of the MID domain or the PAZ domain can be from Ago4.

In some embodiments of any one of the aspects, the MID domain and the PAZ domain are from the same Ago. For example, both of the MID domain and the PAZ domain are from Ago1, from Ago2, from Ago3, or from Ago4. In some embodiments, both of the MID domain and the PAZ domain are from Ago2.

The MID domain and the PAZ domain and can be from the same Ago or different species. For example, the PAZ domain can be from a eukaryotic Ago or a prokaryotic Ago. In some embodiments, the MID domain and the PAZ domain are from different species. In some embodiments, one of the MID domain or the PAZ domain is from a eukaryotic Ago and the other one is from a prokaryotic Ago. In some other embodiments, both the MID domain and the PAZ domain can be from eukaryote Agos. In some embodiments, both the MID domain and the PAZ domain can be from mammalian Agos. For example, both the MID domain and the PAZ domain are from Ago1, from Ago2, from Ago3 or from Ago4.

In some embodiments of any one of the aspects, the second domain of the polypeptide further comprises a PIWI domain of an Ago. It is noted that PIWI domain can from a eukaryotic or prokaryotic Ago. In some embodiments of any one of the aspects, the PIWI domain is from a mammalian Ago such as a mouse or human Ago. For example, the PIWI domain is from human Ago (hAgo) 1, hAgo2, hAgo3 or hAgo4. In some preferred embodiments, the PIWI domain is from hAgo2.

In some embodiments of any one of the aspects, the PIWI domain lack nuclease activity.

The MID domain and the PIWI domain and can be from the same Ago or different Agos. For example, one of the MID domain or the PIWI domain can be from Ago1, and the other of the MID domain or the PIWI domain can be from Ago2, Ago3 or Ago4. In another example, one of the MID domain or the PIWI domain can be from Ago2, and the other of the MID domain or the PIWI domain can be from Ago3 or Ago4. In yet another non-limiting example, one of the MID domain or the PIWI domain can be from Ago3, and the other of the MID domain or the PIWI domain can be from Ago4.

In some embodiments of any one of the aspects, the MID domain and the PIWI domain are from the same Ago. For example, both of the MID domain and the PIWI domain are from Ago1, from Ago2, from Ago3, or from Ago4. In some embodiments, both of the MID domain and the PIWI domain are from Ago2.

The MID domain and the PIWI domain and can be from the same Ago or different species. For example, the PIWI domain can be from a eukaryotic Ago or a prokaryotic Ago. In some embodiments, the MID domain and the PIWI domain are from different species. In some embodiments, one of the MID domain or the PIWI domain is from a eukaryotic Ago and the other one is from a prokaryotic Ago. In some other embodiments, both the MID domain and the PIWI domain can be from eukaryote Agos. In some embodiments, both the MID domain and the PIWI domain can be from mammalian Agos. For example, both the MID domain and the PIWI domain are from Ago1, from Ago2, from Ago3 or from Ago4.

In some embodiments of any one of the aspects, the second domain of the polypeptide comprises a MID domain of an Ago, a PAZ domain of an Ago and a PIWI domain of an Ago.

In some embodiments of any one of the aspects, the second domain of the polypeptide further comprises a N-terminal domain of an Ago. It is noted that N-terminal domain can from a eukaryotic or prokaryotic Ago. In some embodiments of any one of the aspects, the N-terminal domain is from a mammalian Ago such as a mouse or human Ago. For example, the N-terminal domain is from human Ago (hAgo) 1, hAgo2, hAgo3 or hAgo4. In some preferred embodiments, the N-terminal domain is from hAgo2.

In some embodiments of any one of the aspects, the N-terminal domain lacks nuclease activity.

The MID domain and the N-terminal domain and can be from the same Ago or different Agos. For example, one of the MID domain or the N-terminal domain can be from Ago1, and the other of the MID domain or the N-terminal domain can be from Ago2, Ago3 or Ago4. In another example, one of the MID domain or the N-terminal domain can be from Ago2, and the other of the MID domain or the N-terminal domain can be from Ago3 or Ago4. In yet another non-limiting example, one of the MID domain or the N-terminal domain can be from Ago3, and the other of the MID domain or the N-terminal domain can be from Ago4.

In some embodiments of any one of the aspects, the MID domain and the N-terminal domain are from the same Ago. For example, both of the MID domain and the N-TERMINAL domain are from Ago1, from Ago2, from Ago3, or from Ago4. In some embodiments, both of the MID domain and the N-terminal domain are from Ago2.

The MID domain and the N-terminal domain and can be from the same Ago or different species. For example, the N-terminal domain can be from a eukaryotic Ago or a prokaryotic Ago. In some embodiments, the MID domain and the N-terminal domain are from different species. In some embodiments, one of the MID domain or the N-terminal domain is from a eukaryotic Ago and the other one is from a prokaryotic Ago. In some other embodiments, both the MID domain and the N-terminal domain can be from eukaryote Agos. In some embodiments, both the MID domain and the N-terminal domain can be from mammalian Agos. For example, both the MID domain and the N-terminal domain are from Ago1, from Ago2, from Ago3 or from Ago4.

In some embodiments of any one of the aspects, the second domain of the polypeptide comprises a MID domain of an Ago, a PAZ domain of an Ago and an N-terminal domain of an Ago.

In some embodiments of any one of the aspects, the second domain of the polypeptide comprises a MID domain of an Ago, a PIWI domain of an Ago and an N-terminal domain of an Ago.

In some embodiments of any one of the aspects, the second domain of the polypeptide comprises a MID domain of an Ago, a PAZ domain of an Ago, a PIWI domain of an Ago and an N-terminal domain of an Ago.

It is noted the amino acid sequences for the Ago domains, e.g., MID, PAZ, PIWI and/or N-terminal domain can be altered such that they vary sequences from the naturally occurring or native sequences from which they were derived, while retaining the desired activity of the native sequence. Accordingly, in some embodiments of any one of the aspects, an Ago domain in polypeptide, e.g., MID, PAZ, PIWI and/or N-terminal domain comprises an amino acid sequence having at least 80%, 81%, 82%, 83%, 84%, 85%, 86%, 87%, 88%, 89%, 90%, 91%, 92%, 93%, 94%, 95%, 96%, 97%, 98% or 99% identity to a wild-type sequence of said Ago domain.

In some embodiments of any one of the aspects, the second domain comprises an amino acid sequence having at least 80%, 81%, 82%, 83%, 84%, 85%, 86%, 87%, 88%, 89%, 90%, 91%, 92%, 93%, 94%, 95%, 96%, 97%, 98% or 99% identity to a mammalian Ago. For example, the second domain comprises an amino acid sequence having at least 80%, 81%, 82%, 83%, 84%, 85%, 86%, 87%, 88%, 89%, 90%, 91%, 92%, 93%, 94%, 95%, 96%, 97%, 98% or 99% identity to a hAgo1, hAgo2, hAgo3, hAgo4 or a homologous or orthologous Ago protein.

Exemplary sequences for human Argonaute proteins can be found in Genebank with Accession Numbers as listed: hAgo1 isoform 1 (NP 036331.1), hAgo1 isoform 1× (NP 0011304051.1), hAgo1 isoform 2 (NP 001304052.1), hAgo2 isoform 1 (NP 036286.2), hAgo2 isoform 2 (NP 001158095.1), hAgo3 isoform 3 (NP 079128.2), hAgo3 isoform 3 (NP 803171.1), hAgo4 isoform X1 (XP 005270635.1), hAgo4 isoform X2 (XP 011539185.1), hAgo4 isoform X3 (XP 011549186.1) and hAgo4 isoform X4 (XP 024309563.1).

In some embodiments, the second domain comprises an amino acid sequence having at least 80%, 81%, 82%, 83%, 84%, 85%, 86%, 87%, 88%, 89%, 90%, 91%, 92%, 93%, 94%, 95%, 96%, 97%, 98% or 99% identity to an amino acid sequence selected from the group consisting of:

hAgo1:
(SEQ ID NO: 1)
MEAGPSGAAAGAYLPPLQQVFQAPRRPGIGTVGKPIKLLANYFEVDIPKIDVYHYEVDIK
PDKCPRRVNREVVEYMVQHFKPQIFGDRKPVYDGKKNIYTVTALPIGNERVDFEVTIPGE
GKDRIFKVSIKWLAIVSWRMLHEALVSGQIPVPLESVQALDVAMRHLASMRYTPVGRSF
FSPPEGYYHPLGGGREVWFGFHQSVRPAMWKMMLNIDVSATAFYKAQPVIEFMCEVLD
IRNIDEQPKPLTDSQRVRFTKEIKGLKVEVTHCGQMKRKYRVCNVTRRPASHQTFPLQLE
SGQTVECTVAQYFKQKYNLQLKYPHLPCLQVGQEQKHTYLPLEVCNIVAGQRCIKKLTD
NQTSTMIKATARSAPDRQEEISRLMKNASYNLDPYIQEFGIKVKDDMTEVTGRVLPAPIL
QYGGRNRAIATPNQGVWDMRGKQFYNGIEIKVWAIACFAPQKQCREEVLKNFTDQLRK
ISKDAGMPIQGQPCFCKYAQGADSVEPMFRHLKNTYSGLQLIIVILPGKTPVYAEVKRVG
DTLLGMATQCVQVKNVVKTSPQTLSNLCLKINVKLGGINNILVPHQRSAVFQQPVIFLGA
DVTHPPAGDGKKPSITAVVGSMDAHPSRYCATVRVQRPRQEIIEDLSYMVRELLIQFYKS
TRFKPTRIIFYRDGVPEGQLPQILHYELLAIRDACIKLEKDYQPGITYIVVQKRHHTRLFCA
DKNERIGKSGNIPAGTTVDTNITHPFEFDFYLCSHAGIQGTSRPSHYYVLWDDNRFTADE
LQILTYQLCHTYVRCTRSVSIPAPAYYARLVAFRARYHLVDKEHDSGEGSHISGQSNGRD
PQALAKAVQVHQDTLRTMYFA
hAgo2:
(SEQ ID NO: 2)
MYSGAGPALAPPAPPPPIQGYAFKPPPRPDFGTSGRTIKLQANFFEMDIPKIDIYHYELDIK
PEKCPRRVNREIVEHMVQHFKTQIFGDRKPVFDGRKNLYTAMPLPIGRDKVELEVTLPGE
GKDRIFKVSIKWVSCVSLQALHDALSGRLPSVPFETIQALDVVMRHLPSMRYTPVGRSFF
TASEGCSNPLGGGREVWFGFHQSVRPSLWKMMLNIDVSATAFYKAQPVIEFVCEVLDFK
SIEEQQKPLTDSQRVKFTKEIKGLKVEITHCGQMKRKYRVCNVTRRPASHQTFPLQQESG
QTVECTVAQYFKDRHKLVLRYPHLPCLQVGQEQKHTYLPLEVCNIVAGQRCIKKLTDNQ
TSTMIRATARSAPDRQEEISKLMRSASFNTDPYVREFGIMVKDEMTDVTGRVLQPPSILY
GGRNKAIATPVQGVWDMRNKQFHTGIEIKVWAIACFAPQRQCTEVHLKSFTEQLRKISR
DAGMPIQGQPCFCKYAQGADSVEPMFRHLKNTYAGLQLVVVILPGKTPVYAEVKRVGD
TVLGMATQCVQMKNVQRTTPQTLSNLCLKINVKLGGVNNILLPQGRPPVFQQPVIFLGA
DVTHPPAGDGKKPSIAAVVGSMDAHPNRYCATVRVQQHRQEIIQDLAAMVRELLIQFYK
STRFKPTRIIFYRDGVSEGQFQQVLHHELLAIREACIKLEKDYQPGITFIVVQKRHHTRLFC
TDKNERVGKSGNIPAGTTVDTKITHPTEFDFYLCSHAGIQGTSRPSHYHVLWDDNRFSSD
ELQILTYQLCHTYVRCTRSVSIPAPAYYAHLVAFRARYHLVDKEHDSAEGSHTSGQSNG
RDHQALAKAVQVHQDTLRTMYFA
hAgo3:
(SEQ ID NO: 3)
MEIGSAGPAGAQPLLMVPRRPGYGTMGKPIKLLANCFQVEIPKIDVYLYEVDIKPDKCPR
RVNREVVDSMVQHFKVTIFGDRRPVYDGKRSLYTANPLPVATTGVDLDVTLPGEGGKD
RPFKVSIKFVSRVSWHLLHEVLTGRTLPEPLELDKPISTNPVHAVDVVLRHLPSMKYTPV
GRSFFSAPEGYDHPLGGGREVWFGFHQSVRPAMWKMMLNIDVSATAFYKAQPVIQFMC
EVLDIHNIDEQPRPLTDSHRVKFTKEIKGLKVEVTHCGTMRRKYRVCNVTRRPASHQTFP
LQLENGQTVERTVAQYFREKYTLQLKYPHLPCLQVGQEQKHTYLPLEVCNIVAGQRCIK
KLTDNQTSTMIKATARSAPDRQEEISRLVRSANYETDPFVQEFQFKVRDEMAHVTGRVL
PAPMLQYGGRNRTVATPSHGVWDMRGKQFHTGVEIKMWAIACFATQRQCREEILKGFT
DQLRKISKDAGMPIQGQPCFCKYAQGADSVEPMFRHLKNTYSGLQLIIVILPGKTPVYAE
VKRVGDTLLGMATQCVQVKNVIKTSPQTLSNLCLKINVKLGGINNILVPHQRPSVFQQPV
IFLGADVTHPPAGDGKKPSIAAVVGSMDAHPSRYCATVRVQRPRQEIIQDLASMVRELLI
QFYKSTRFKPTRIIFYRDGVSEGQFRQVLYYELLAIREACISLEKDYQPGITYIVVQKRHHT
RLFCADRTERVGRSGNIPAGTTVDTDITHPYEFDFYLCSHAGIQGTSRPSHYHVLWDDNC
FTADELQLLTYQLCHTYVRCTRSVSIPAPAYYAHLVAFRARYHLVDKEHDSAEGSHVSG
QSNGRDPQALAKAVQIHQDTLRTMYFA
hAgo4:
(SEQ ID NO: 4)
MEALGPGPPASLFQPPRRPGLGTVGKPIRLLANHFQVQIPKIDVYHYDVDIKPEKRPRRV
NREVVDTMVRHFKMQIFGDRQPGYDGKRNMYTAHPLPIGRDRVDMEVTLPGEGKDQT
FKVSVQWVSVVSLQLLLEALAGHLNEVPDDSVQALDVITRHLPSMRYTPVGRSFFSPPEG
YYHPLGGGREVWFGFHQSVRPAMWNMMLNIDVSATAFYRAQPIIEFMCEVLDIQNINEQ
TKPLTDSQRVKFTKEIRGLKVEVTHCGQMKRKYRVCNVTRRPASHQTFPLQLENGQAM
ECTVAQYFKQKYSLQLKYPHLPCLQVGQEQKHTYLPLEVCNIVAGQRCIKKLTDNQTST
MIKATARSAPDRQEEISRLVKSNSMVGGPDPYLKEFGIVVHNEMTELTGRVLPAPMLQY
GGRNKTVATPNQGVWDMRGKQFYAGIEIKVWAVACFAPQKQCREDLLKSFTDQLRKIS
KDAGMPIQGQPCFCKYAQGADSVEPMFKHLKMTYVGLQLIVVILPGKTPVYAEVKRVG
DTLLGMATQCVQVKNVVKTSPQTLSNLCLKINAKLGGINNVLVPHQRPSVFQQPVIFLG
ADVTHPPAGDGKKPSIAAVVGSMDGHPSRYCATVRVQTSRQEISQELLYSQEVIQDLTN
MVRELLIQFYKSTRFKPTRIIYYRGGVSEGQMKQVAWPELIAIRKACISLEEDYRPGITYIV
VQKRHHTRLFCADKTERVGKSGNVPAGTTVDSTITHPSEFDFYLCSHAGIQGTSRPSHYQ
VLWDDNCFTADELQLLTYQLCHTYVRCTRSVSIPAPAYYARLVAFRARYHLVDKDHDS
AEGSHVSGQSNGRDPQALAKAVQIHHDTQHTMYFA

In some embodiments, the second domain comprises an amino acid sequence having at least 90%, 91%, 92%, 93%, 94%, 95%, 96%, 97%, 98% or 99% identity to an amino acid sequence selected from the group consisting of SEQ ID NO: 1-4. In some embodiments, the second domain comprises an amino acid sequence having at least 95%, 96%, 97%, 98% or 99% identity to an amino acid sequence selected from the group consisting of SEQ ID NO: 1-4. In some embodiments, the second domain comprises an amino acid sequence having at least 97%, 98% or 99% identity to an amino acid sequence selected from the group consisting of SEQ ID NO: 1-4. In some embodiments, the second domain comprises an amino acid sequence having 100% identity to an amino acid sequence selected from the group consisting of SEQ ID NO: 1-4.

In some preferred embodiments, the second domain comprises the amino acid sequence of SEQ ID NO: 2.

In some embodiments, the second domain comprises an amino acid sequence having a mutation at one or more positions selected from the group consisting of D597 and D699 of human Ago2 amino acid sequence, e.g., SEQ ID NO: 2, or a corresponding position in a homologous or orthologous Ago protein. For example, the second domain comprises an amino acid sequence having a mutation at one or more positions selected from the group consisting of D597A and D699A of human Ago2 amino acid sequence, e.g., SEQ ID NO: 2, or a corresponding position in a homologous or orthologous Ago protein.

### Catalytic Domain

In some embodiments of any one of the aspects, the polypeptide comprises a catalytic domain from an RNA modifying enzyme. Generally, the catalytic domain lacks nuclease activity. RNA modifying enzymes amenable to the systems, compositions, kits and methods described herein include, but are not limited to, those that can edit a nucleotide or ribonucleotide (e.g., adenosine deaminases, ADAR family proteins, cytidine deaminases, APOBEC family proteins, and PPR proteins), those that can methylate RNA (e.g., domains from m6A methyltransferase factors such as METTL3, METTL4, METTL14, or WTAP), those that can demethylate RNA (e.g., human alkylation repair homolog 5 or ALKBH5), those that can affect splicing (e.g., the RS-rich domain of SRSF1, the Gly-rich domain of hnRNP A1, the alanine-rich motif of RBM4, or the proline-rich motif of DAZAP1), those that can activate translation (e.g., eIF4E, N-terminal domain of the YT521-B homolog domain family protein 1 (YTHDF1, a cytoplasmicm6A reader protein that recruits the translation machinery) and other translation initiation factors, a domain of the yeast poly(A)-binding protein or GLD2), those that can repress translation (e.g., Pumilio or FBF PUF proteins, deadenylases, or CAF1) and those that can affect RNA stability (e.g., tristetraprolin (TTP) or domains from UPF1, EXOSC5, and STAU1).

In some embodiments of any one of the aspects, the catalytic domain of an RNA modifying enzyme is a deaminase domain of a deaminase. For example, the catalytic domain is a deaminase domain of an adenosine deaminase, a cytidine deaminase, an apolipoprotein B mRNA-editing complex (APOBEC) family deaminase, an activation-induced cytidine deaminase (AID), an ACF1/ASE deaminase or an ADAT family deaminase. It should be appreciated that the deaminase may be from any suitable organism (e.g., a human or a rat). In some embodiments, the deaminase is from a human, chimpanzee, gorilla, monkey, cow, dog, rat, or mouse.

Adenosine deaminases that can be used in connection with the present disclosure include, but are not limited to, members of the enzyme family known as adenosine deaminases that act on RNA (ADARs), members of the enzyme family known as adenosine deaminases that act on tRNA (ADATs), and other adenosine deaminase domain-containing (ADAD) family member. In some embodiments of any one of the aspects, the deaminase domain is from ADAR.

ADAR is an adenosine deaminase that specifically recognizes the double-stranded part of RNA and converts adenosine residues into inosine. The ADAR can be a mammalian ADAR. There are three mammalian ADARs denoted as ADAR1, ADAR2, and ADAR3. ADAT1 is another enzyme that converts adenosine residues into inosine. ADAR1 isoforms and ADAR2 are widely expressed in a variety of cells and tissues with the highest expression in the brain and spleen and are the essential ADARs involved in 5HTR2C mRNA editing.

In some embodiments, the adenosine deaminase is a human ADAR, including hADAR1, hADAR2, hADAR3. In some embodiments, the adenosine deaminase is a Caenorhabditis elegans ADAR protein, including ADR-1 and ADR-2. In some embodiments, the adenosine deaminase is a Drosophila ADAR protein, including dAdar. In some embodiments, the adenosine deaminase is a squid Loligo pealeii ADAR protein, including sqADAR2a and sqADAR2b. In some embodiments, the adenosine deaminase is a human ADAT protein. In some embodiments, the adenosine deaminase is a Drosophila ADAT protein. In some embodiments, the adenosine deaminase is a human ADAD protein, including TENR (hADAD1) and TENRL (hADAD2).

In some embodiments, the first domain comprises an amino acid sequence having at least 80%, 81%, 82%, 83%, 84%, 85%, 86%, 87%, 88%, 89%, 90%, 91%, 92%, 93%, 94%, 95%, 96%, 97%, 98% or 99% identity to a wild-type sequence of ADAR. For example, the first domain comprises an amino acid sequence having at least 80%, 81%, 82%, 83%, 84%, 85%, 86%, 87%, 88%, 89%, 90%, 91%, 92%, 93%, 94%, 95%, 96%, 97%, 98% or 99% identity to a human ADAR or a homologous or orthologous ADAR.

Exemplary sequences for human ADAR proteins can be found in Genebank with Accession Numbers as listed: hADAR1 isoform a (NP 001102.3), hADAR1 isoform b (NP 056655.3), hADAR1 isoform c (NP 056656.3), hADAR1 isoform d (NP 001020278.1), hADAR1 isoform e (NP 001351974.1), hADAR1 isoform f (NP 001351978.1), hADAR2 isoform 1 (NP 001103.1), hADAR2 isoform 2 (NP 056648.1), hADAR2 isoform 3 (NP 056649.1), hADAR2 isoform 7 (NP 001153702.1), hADAR2 isoform 8 (NP 001333616.1) and hADAR3 (NP 061172.1).

In some embodiments of any one of the aspects, the catalytic domain is a deaminase domain of hADAR1, hADAR2 or hADAR3. For example, the first domain comprises an amino acid sequence having at least 80%, 81%, 82%, 83%, 84%, 85%, 86%, 87%, 88%, 89%, 90%, 91%, 92%, 93%, 94%, 95%, 96%, 97%, 98% or 99% identity to hADAR1, hADAR2, hADAR3 or a homologous or orthologous ADAR. Preferably, the first domain comprises an amino acid sequence having at least 80%, 81%, 82%, 83%, 84%, 85%, 86%, 87%, 88%, 89%, 90%, 91%, 92%, 93%, 94%, 95%, 96%, 97%, 98% or 99% identity to hADAR2.

A deaminase domain for use in the polypeptide can be from a modified ADAR. Accordingly, in some embodiments, the first domain comprises an amino acid sequence having a mutation at one or more positions selected from the group consisting of T375, and E488 of human ADAR2 (hADAR2) amino acid sequence, or a corresponding position in a homologous or orthologous ADAR protein. For example, the first domain comprises an amino acid sequence having a mutation at one or more positions selected from the group consisting of T375Q, and E488Q of human ADAR2 (hADAR2) amino acid sequence, or a corresponding position in a homologous or orthologous ADAR protein.

In some embodiments, the first domain comprises an amino acid sequence having a mutation at one or more positions selected from the group consisting of G336, T339, R348, A353, V351, V355, T375, K376, E396, S397, E438, F442, H443, L444, Y445, T448, T490, C451, R455, S486, G487, Q488, R510, I520, V525, P539, G593 and K594 of hADAR2 amino acid sequence, or a corresponding position in a homologous or orthologous ADAR protein. For example, the first domain comprises an amino acid sequence having a mutation at one or more positions selected from the group consisting of V351, S370, T375, P462, S486, E488 and N597 of hADAR2 amino acid sequence, or a corresponding position in a homologous or orthologous ADAR protein.

In some embodiments, the first domain comprises an amino acid sequence having a mutation at G336 of hADAR2 amino acid sequence, or a corresponding position in a homologous or orthologous ADAR protein. For example, the first domain comprises an amino acid sequence having a mutation selected from the group consisting of G336D, G487A, G487V, G487R, G487K, G487W, and G487Y of hADAR2 amino acid sequence, or a corresponding position in a homologous or orthologous ADAR protein.

In some embodiments, the first domain comprises an amino acid sequence having a mutation at E488 of hADAR2 amino acid sequence, or a corresponding position in a homologous or orthologous ADAR protein. For example, the first domain comprises an amino acid sequence having a mutation selected from the group consisting of E488Q, E488R, E488R, E488K, E488N, E488A, E488M, E488S, E488F, E488L and E488W of hADAR2 amino acid sequence, or a corresponding position in a homologous or orthologous ADAR protein.

In some embodiments, the first domain comprises an amino acid sequence having a mutation at T490 of hADAR2 amino acid sequence, or a corresponding position in a homologous or orthologous ADAR protein. For example, the first domain comprises an amino acid sequence having a mutation selected from the group consisting of T490C, T490S, T490A, T490F, 490Y, T490R, T490K, T490P and T490E of hADAR2 amino acid sequence, or a corresponding position in a homologous or orthologous ADAR protein.

In some embodiments, the first domain comprises an amino acid sequence having a mutation at V493 of hADAR2 amino acid sequence, or a corresponding position in a homologous or orthologous ADAR protein. For example, the first domain comprises an amino acid sequence having a mutation selected from the group consisting of V493A, V493S, V493T, V493R, V493D, V493P and V493G of hADAR2 amino acid sequence, or a corresponding position in a homologous or orthologous ADAR protein.

In some embodiments, the first domain comprises an amino acid sequence having a mutation at A589 of hADAR2 amino acid sequence, or a corresponding position in a homologous or orthologous ADAR protein. For example, the first domain comprises an amino acid sequence having a A589V mutation in the hADAR2 amino acid sequence, or a corresponding position in a homologous or orthologous ADAR protein.

In some embodiments, the first domain comprises an amino acid sequence having a mutation at N597 of hADAR2 amino acid sequence, or a corresponding position in a homologous or orthologous ADAR protein. For example, the first domain comprises an amino acid sequence having a mutation selected from the group consisting of N597K, N597R, N597A, N597E, N597H, N597G, N597Y and N597F of hADAR2 amino acid sequence, or a corresponding position in a homologous or orthologous ADAR protein.

In some embodiments, the first domain comprises an amino acid sequence having a mutation at S599 of hADAR2 amino acid sequence, or a corresponding position in a homologous or orthologous ADAR protein. For example, the first domain comprises an amino acid sequence having a S599T mutation in the hADAR2 amino acid sequence, or a corresponding position in a homologous or orthologous ADAR protein.

In some embodiments, the first domain comprises an amino acid sequence having a mutation at N613 of hADAR2 amino acid sequence, or a corresponding position in a homologous or orthologous ADAR protein. For example, the first domain comprises an amino acid sequence having a mutation selected from the group consisting of N613K, N613R, N613A and N613E of hADAR2 amino acid sequence, or a corresponding position in a homologous or orthologous ADAR protein.

In some embodiments, to improve the deamination activity of the ADAR, the first domain comprises an amino acid sequence having a mutation at one or more positions selected from the group consisting of G336D, G487A, G487V, E488Q, E488H, E488R, E488N, E488A, E488S, E488M, T490C, T490S, V493T, V493S, V493A, V493R, V493D, V493P, V493G, N597K, N597R, N597A, N597E, N597H, N597G, N597Y, A589V, S599T, N613K, N613R, N613A and N613E of hADAR2 amino acid sequence, or a corresponding position in a homologous or orthologous ADAR protein.

At times, there may be a need, to reduce the deamination activity of the ADAR. Accordingly, in some embodiments, the first domain comprises an amino acid sequence having a mutation at one or more positions selected from the group consisting of E488F, E488L, E488W, T490A, T490F, T490Y, T490R, T490K, T490P, T490E and N597F of hADAR2 amino acid sequence, or a corresponding position in a homologous or orthologous ADAR protein.

In some embodiments, the first domain comprises an amino acid sequence having a mutation at one or more positions selected from the group consisting of R348, V351, T375, K376, E396, C451, R455, N473, R474, K475, R477, R481, S486, E488, T490, S495 and R510 of hADAR2, or a corresponding position in a homologous or orthologous ADAR protein. For example, the first domain comprises an amino acid sequence having a mutation at position E488 and a mutation at one or more positions selected from the group consisting of R348, V351, T375, K376, E396, C451, R455, N473, R474, K475, R477, R481, S486, T490, S495 and R510 of hADAR2, or a corresponding position in a homologous or orthologous ADAR protein.

In some embodiments, the first domain comprises an amino acid sequence having a mutation selected from the group consisting of R348E, V351L, T375G, T375S, R455G, R455S, R455E, N473D, R474E, K475Q, R477E, R481E, S486T, E488Q, T490A, T490S, S495T and R510E of hADAR2, or a corresponding position in a homologous or orthologous ADAR protein. For example, the first domain comprises an amino acid sequence having the mutation E488Q and a mutation selected from the group consisting of R348E, V351L, T375G, T375S, R455G, R455S, R455E, N473D, R474E, K475Q, R477E, R481E, S486T, T490A, T490S, S495T and R510E of hADAR2, or a corresponding position in a homologous or orthologous ADAR protein. In some embodiments, the first domain comprises an amino acid sequence having a mutation at position G1007 of hADAR1, or a corresponding position in a homologous or orthologous ADAR protein. For example, the first domain comprises an amino acid sequence having a mutation selected from the group consisting of G1007A, G1007V, G1007R, G1007K, G1007W, G1007Y, G1007L, G1007T and G1007S of hADAR1, or a corresponding position in a homologous or orthologous ADAR protein.

In some embodiments, the first domain comprises an amino acid sequence having a mutation at position E1008 of hADAR1, or a corresponding position in a homologous or orthologous ADAR protein. For example, the first domain comprises an amino acid sequence having a mutation selected from the group consisting of E1008Q, E1008H, E1008R, E1008K, E1008F, E1008W, E1008G, E1008I, E1008V, E1008P, E1008S, E1008N, E1008A, E1008M and E1008L of hADAR1, or a corresponding position in a homologous or orthologous ADAR protein.

In some embodiments, the first domain comprises an amino acid sequence having at least 80%, 81%, 82%, 83%, 84%, 85%, 86%, 87%, 88%, 89%, 90%, 91%, 92%, 93%, 94%, 95%, 96%, 97%, 98% or 99% identity to an amino acid sequence selected from the group consisting of:

hADAR1:
(SEQ ID NO: 5)
MNPRQGYSLSGYYTHPFQGYEHRQLRYQQPGPGSSPSSFLLKQIEFLKGQLPEAPVIGKQ
TPSLPPSLPGLRPRFPVLLASSTRGRQVDIRGVPRGVHLRSQGLQRGFQHPSPRGRSLPQR
GVDCLSSHFQELSIYQDQEQRILKFLEELGEGKATTAHDLSGKLGTPKKEINRVLYSLAK
KGKLQKEAGTPPLWKIAVSTQAWNQHSGVVRPDGHSQGAPNSDPSLEPEDRNSTSVSED
LLEPFIAVSAQAWNQHSGVVRPDSHSQGSPNSDPGLEPEDSNSTSALEDPLEFLDMAEIKE
KICDYLFNVSDSSALNLAKNIGLTKARDINAVLIDMERQGDVYRQGTTPPIWHLTDKKRE
RMQIKRNTNSVPETAPAAIPETKRNAEFLTCNIPTSNASNNMVTTEKVENGQEPVIKLEN
RQEARPEPARLKPPVHYNGPSKAGYVDFENGQWATDDIPDDLNSIRAAPGEFRAIMEMP
SFYSHGLPRCSPYKKLTECQLKNPISGLLEYAQFASQTCEFNMIEQSGPPHEPRFKFQVVI
NGREFPPAEAGSKKVAKQDAAMKAMTILLEEAKAKDSGKSEESSHYSTEKESEKTAESQ
TPTPSATSFFSGKSPVTTLLECMHKLGNSCEFRLLSKEGPAHEPKFQYCVAVGAQTFPSVS
APSKKVAKQMAAEEAMKALHGEATNSMASDNQPEGMISESLDNLESMMPNKVRKIGEL
VRYLNTNPVGGLLEYARSHGFAAEFKLVDQSGPPHEPKFVYQAKVGGRWFPAVCAHSK
KQGKQEAADAALRVLIGENEKAERMGFTEVTPVTGASLRRTMLLLSRSPEAQPKTLPLT
GSTFHDQIAMLSHRCFNTLTNSFQPSLLGRKILAAIIMKKDSEDMGVVVSLGTGNRCVKG
DSLSLKGETVNDCHAEIISRRGFIRFLYSELMKYNSQTAKDSIFEPAKGGEKLQIKKTVSF
HLYISTAPCGDGALFDKSCSDRAMESTESRHYPVFENPKQGKLRTKVENGEGTIPVESSDI
VPTWDGIRLGERLRTMSCSDKILRWNVLGLQGALLTHFLQPIYLKSVTLGYLFSQGHLTR
AICCRVTRDGSAFEDGLRHPFIVNHPKVGRVSIYDSKRQSGKTKETSVNWCLADGYDLEI
LDGTRGTVDGPRNELSRVSKKNIFLLFKKLCSFRYRRDLLRLSYGEAKKAARDYETAKN
YFKKGLKDMGYGNWISKPQEEKNFYLCPV
hADAR2
(SEQ ID NO: 6)
MDIEDEENMSSSSTDVKENRNLDNVSPKDGSTPGPGEGSQLSNGGGGGPGRKRPLEEGS
NGHSKYRLKKRRKTPGPVLPKNALMQLNEIKPGLQYTLLSQTGPVHAPLFVMSVEVNG
QVFEGSGPTKKKAKLHAAEKALRSFVQFPNASEAHLAMGRTLSVNTDFTSDQADFPDTL
FNGFETPDKAEPPFYVGSNGDDSFSSSGDLSLSASPVPASLAQPPLPVLPPFPPPSGKNPVM
ILNELRPGLKYDELSESGESHAKSFVMSVVVDGQFFEGSGRNKKLAKARAAQSALAAIF
NLHLDQTPSRQPIPSEGLQLHLPQVLADAVSRLVLGKFGDLTDNFSSPHARRKVLAGVV
MTTGTDVKDAKVISVSTGTKCINGEYMSDRGLALNDCHAEIISRRSLLRFLYTQLELYLN
NKDDQKRSIFQKSERGGFRLKENVQFHLYISTSPCGDARIFSPHEPILEEPADRHPNRKAR
GQLRTKIESGEGTIPVRSNASIQTWDGVLQGERLLTMSCSDKIARWNVVGIQGSLLSIFVE
PIYFSSIILGSLYHGDHLSRAMYQRISNIEDLPPLYTLNKPLLSGISNAEARQPGKAPNFSV
NWTVGDSAIEVINATTGKDELGRASRLCKHALYCRWMRVHGKVPSHLLRSKITKPNVY
HESKLAAKEYQAAKARLFTAFIKAGLGAWVEKPTEQDQFSLTP
hADAR3:
(SEQ ID NO: 7)
MASVLGSGRGSGGLSSQLKCKSKRRRRRRSKRKDKVSILSTFLAPFKHLSPGITNTEDDD
TLSTSSAEVKENRNVGNLAARPPPSGDRARGGAPGAKRKRPLEEGNGGHLCKLQLVWK
KLSWSVAPKNALVQLHELRPGLQYRTVSQTGPVHAPVFAVAVEVNGLTFEGTGPTKKK
AKMRAAELALRSFVQFPNACQAHLAMGGGPGPGTDFTSDQADFPDTLFQEFEPPAPRPG
LAGGRPGDAALLSAAYGRRRLLCRALDLVGPTPATPAAPGERNPVVLLNRLRAGLRYV
CLAEPAERRARSFVMAVSVDGRTFEGSGRSKKLARGQAAQAALQELFDIQMPGHAPGR
ARRTPMPQEFADSISQLVTQKFREVTTDLTPMHARHKALAGIVMTKGLDARQAQVVAL
SSGTKCISGEHLSDQGLVVNDCHAEVVARRAFLHFLYTQLELHLSKRREDSERSIFVRLK
EGGYRLRENILFHLYVSTSPCGDARLHSPYEITTDLHSSKHLVRKFRGHLRTKIESGEGTV
PVRGPSAVQTWDGVLLGEQLITMSCTDKIARWNVLGLQGALLSHFVEPVYLQSIVVGSL
HHTGHLARVMSHRMEGVGQLPASYRHNRPLLSGVSDAEARQPGKSPPFSMNWVVGSA
DLEIINATTGRRSCGGPSRLCKHVLSARWARLYGRLSTRTPSPGDTPSMYCEAKLGAHTY
QSVKQQLFKAFQKAGLGTWVRKPPEQQQFLLTL

In some embodiments, the first domain comprises an amino acid sequence having at least 90%, 91%, 92%, 93%, 94%, 95%, 96%, 97%, 98% or 99% identity to an amino acid sequence selected from the group consisting of SEQ ID NO: 5-7. In some embodiments, the first domain comprises an amino acid sequence having at least 95%, 96%, 97%, 98% or 99% identity to an amino acid sequence selected from the group consisting of SEQ ID NO: 5-7. In some embodiments, the first domain comprises an amino acid sequence having at least 97%, 98% or 99% identity to an amino acid sequence selected from the group consisting of SEQ ID NO: 5-7. In some embodiments, the first domain comprises an amino acid sequence having 100% identity to an amino acid sequence selected from the group consisting of SEQ ID NO: 5-7.

In some preferred embodiments, the first domain comprises the amino acid sequence of SEQ ID NO: 6.

In some embodiments, the first domain comprises an amino acid sequence having a mutation at one or more positions selected from the group consisting of G336, T339, R348, A353, V351, V355, T375, K376, E396, S397, E438, F442, H443, L444, Y445, T448, T490, C451, R455, S486, G487, Q488, R510, I520, V525, P539, G593 and K594 of hADAR2 amino acid sequence, e.g., SEQ ID NO: 6, or a corresponding position in a homologous or orthologous ADAR protein. For example, the first domain comprises an amino acid sequence having one or more mutations selected from the group consisting G336D, G487A, G487V, E488Q, E488H, E488R, E488N, E488A, E488S, E488M, T490C, T490S, V493T, V493S, V493A, V493R, V493D, V493P, V493G, N597K, N597R, N597A, N597E, N597H, N597G, N597Y, A589V, S599T, N613K, N613R, N613A and N613E of hADAR2 amino acid sequence, or a corresponding position in a homologous or orthologous ADAR protein.

In some embodiments, the first domain comprises an amino acid sequence having a mutation at one or more positions selected from the group consisting of T375, E448 and E488 of hADAR2 amino acid sequence, e.g., SEQ ID NO: 6 or a corresponding position in a homologous or orthologous ADAR protein. For example, the first domain comprises an amino acid sequence having a mutation selected from the group consisting of T375Q, E448Q and E488Q of hADAR2 amino acid sequence, e.g., SEQ ID NO: 6, or a corresponding position in a homologous or orthologous ADAR protein.

In some embodiments, the first domain comprises an amino acid sequence having a mutation at one or more positions selected from the group consisting of G1007 and E1008 of hADAR1 amino acid sequence, e.g., SEQ ID NO: 5, or a corresponding position in a homologous or orthologous ADAR. For example, the first domain comprises an amino acid sequence having a mutation at one or more positions selected from the group consisting of G1007A, G1007V, G1007R, G1007K, G1007W, G1007Y, G1007L, G1007T and G1007S of hADAR1 amino acid sequence, e.g., SEQ ID NO: 5, or a corresponding position in an ADAR protein. In some embodiments, the first domain comprises an amino acid sequence having a mutation at one or more positions selected from the group consisting of E1008Q, E1008H, E1008R, E1008K, E1008F, E1008W, E1008G, E1008I, E1008V, E1008P, E1008S, E1008N, E1008A, E1008M and E1008L of hADAR1 amino acid sequence, e.g., SEQ ID NO: 5, or a corresponding position in an ADAR protein.

In some embodiments, the deaminase domain is from a cytosine deaminase or a cytidine deaminase. In some embodiments, the deaminase is an apolipoprotein B mRNA-editing complex (APOBEC) family deaminase. For example, the deaminase domain is from APOBEC1, APOBEC2, APOBEC3A, APOBEC3B, APOBEC3C, APOBEC3D, APOBEC3F, APOBEC3G, APOBEC3H, or APOBEC4 deaminase. In some embodiments, the deaminase is an activation-induced deaminase (AID).

In some embodiments, the first domain comprises an amino acid sequence having at least 80%, 81%, 82%, 83%, 84%, 85%, 86%, 87%, 88%, 89%, 90%, 91%, 92%, 93%, 94%, 95%, 96%, 97%, 98% or 99% identity to a wild-type sequence of an APOBEC. For example, the first domain comprises an amino acid sequence having at least 80%, 81%, 82%, 83%, 84%, 85%, 86%, 87%, 88%, 89%, 90%, 91%, 92%, 93%, 94%, 95%, 96%, 97%, 98% or 99% identity to a human APOBEC or a homologous or orthologous APOBEC.

Exemplary sequences for human APOBEC proteins can be found in Genebank with Accession Numbers as listed: APOBEC1 isoform a (NP 001291495.1), APOBEC1 isoform b (NP005880), APOBEC2 (NP 006780.1), APOBEC3A isoform a (NP 663745), APOBEC3A isoform b (NP 001257335), APOBEC3B isoform a (NP 004891.5), APOBEC3B isoform b (NP 001257340.2), APOBEC3C (NP 055323), APOBEC3D isoform 1 (NP 689639.2), APOBEC3D isoform 2 (NP 001350710), APOBEC3F isoform a (NP 660341.2), APOBEC3F (NP 001006667.1), APOBEC3G isoform 1 (NP 068594.1), APOBEC3G isoform 2 (NP 001336365.1), APOBEC3G isoform 3 (NP 001336366.1), APOBEC3G isoform 4 (NP 001336367.1), and APOBECC4 (NP 982279.1).

In some embodiments, the first domain comprises an amino acid sequence having at least 80%, 81%, 82%, 83%, 84%, 85%, 86%, 87%, 88%, 89%, 90%, 91%, 92%, 93%, 94%, 95%, 96%, 97%, 98% or 99% identity to an amino acid sequence selected from the group consisting of:

APOBEC1:
(SEQ ID NO: 8)
MTSEKGPSTGDPTLRRRIEPWEFDVFYDPRELRKEACLLYEIKWGMSRKIWRSSGKNTTN
HVEVNFIKKFTSERDFHPSMSCSITWFLSWSPCWECSQAIREFLSRHPGVTLVIYVARLFW
HMDQQNRQGLRDLVNSGVTIQIMRASEYYHCWRNFVNYPPGDEAHWPQYPPLWMML
YALELHCIILSLPPCLKISRRWQNHLTFFRLHLQNCHYQTIPPHILLATGLIHPSVAWR
APOBEC2:
(SEQ ID NO: 9)
MAQKEEAAVATEAASQNGEDLENLDDPEKLKELIELPPFEIVTGERLPANFFKFQFRNVE
YSSGRNKTFLCYVVEAQGKGGQVQASRGYLEDEHAAAHAEEAFFNTILPAFDPALRYN
VTWYVSSSPCAACADRIIKTLSKTKNLRLLILVGRLFMWEEPEIQAALKKLKEAGCKLRI
MKPQDFEYVWQNFVEQEEGESKAFQPWEDIQENFLYYEEKLADILK
APOBEC3A:
(SEQ ID NO: 10)
MEASPASGPRHLMDPHIFTSNFNNGIGRHKTYLCYEVERLDNGTSVKMDQHRGFLHNQ
AKNLLCGFYGRHAELRFLDLVPSLQLDPAQIYRVTWFISWSPCFSWGCAGEVRAFLQEN
THVRLRIFAARIYDYDPLYKEALQMLRDAGAQVSIMTYDEFKHCWDTFVDHQGCPFQP
WDGLDEHSQALSGRLRAILQNQGN
APOBEC3B:
(SEQ ID NO: 11)
MNPQIRNPMERMYRDTFYDNFENEPILYGRSYTWLCYEVKIKRGRSNLLWDTGVFRGQ
VYFKPQYHAEMCFLSWFCGNQLPAYKCFQITWFVSWTPCPDCVAKLAEFLSEHPNVTLT
ISAARLYYYWERDYRRALCRLSQAGARVTIMDYEEFAYCWENFVYNEGQQFMPWYKF
DENYAFLHRTLKEILRYLMDPDTFTFNFNNDPLVLRRRQTYLCYEVERLDNGTWVLMD
QHMGFLCNEAKNLLCGFYGRHAELRFLDLVPSLQLDPAQIYRVTWFISWSPCFSWGCAG
EVRAFLQENTHVRLRIFAARIYDYDPLYKEALQMLRDAGAQVSIMTYDEFEYCWDTFVY
RQGCPFQPWDGLEEHSQALSGRLRAILQNQGN
APOBEC3C:
(SEQ ID NO: 12)
MNPQIRNPMKAMYPGTFYFQFKNLWEANDRNETWLCFTVEGIKRRSVVSWKTGVFRN
QVDSETHCHAERCFLSWFCDDILSPNTKYQVTWYTSWSPCPDCAGEVAEFLARHSNVNL
TIFTARLYYFQYPCYQEGLRSLSQEGVAVEIMDYEDFKYCWENFVYNDNEPFKPWKGLK
TNFRLLKRRLRESLQ
APOBEC3D:
(SEQ ID NO: 114)
MNPQIRNPMERMYRDTFYDNFENEPILYGRSYTWLCYEVKIKRGRSNLLWDTGVFRGPV
LPKRQSNHRQEVDPETHCHAERCFLSWFCDDILSPNTNYEVTWYTSWSPCPECAGEVAE
FLARHSNVNLTIFTARLCYFWDTDYQEGLCSLSQEGASVKIMGYKDFVSCWKNFVYSDD
EPFKPWKGLQTNFRLLKRRLREILQ
APOBEC3F:
(SEQ ID NO: 13)
MKPHFRNTVERMYRDTFSYNFYNRPILSRRNTVWLCYEVKTKGPSRPRLDAKIFRGQVY
SQPEHHAEMCFLSWFCGNQLPAYKCFQITWFVSWTPCPDCVAKLAEFLAEHPNVTLTIS
AARLYYYWERDYRRALCRLSQAGARVKIMDDEEFAYCWENFVYSEGQPFMPWYKFDD
NYAFLHRTLKEILRNPMEAMYPHIFYFHFKNLRKAYGRNESWLCFTMEVVKHHSPVSW
KRGVFRNQVDPETHCHAERCFLSWFCDDILSPNTNYEVTWYTSWSPCPECAGEVAEFLA
RHSNVNLTIFTARLYYFWDTDYQEGLRSLSQEGASVEIMGYKDFKYCWENFVYNDDEPF
KPWKGLKYNFLFLDSKLQEILE
APOBEC3G:
(SEQ ID NO: 14)
MKPHFRNTVERMYRDTFSYNFYNRPILSRRNTVWLCYEVKTKGPSRPPLDAKIFRGQVY
SELKYHPEMRFFHWFSKWRKLHRDQEYEVTWYISWSPCTKCTRDMATFLAEDPKVTLTI
FVARLYYFWDPDYQEALRSLCQKRDGPRATMKIMNYDEFQHCWSKFVYSQRELFEPW
NNLPKYYILLHIMLGEILRHSMDPPTFTFNFNNEPWVRGRHETYLCYEVERMHNDTWVL
LNQRRGFLCNQAPHKHGFLEGRHAELCFLDVIPFWKLDLDQDYRVTCFTSWSPCFSCAQ
EMAKFISKNKHVSLCIFTARIYDDQGRCQEGLRTLAEAGAKISIMTYSEFKHCWDTFVDH
QGCPFQPWDGLDEHSQDLSGRLRAILQNQEN
APOBEC3H:
(SEQ ID NO: 15)
MALLTAETFRLQFNNKRRLRRPYYPRKALLCYQLTPQNGSTPTRGYFENKKKCHAEICFI
NEIKSMGLDETQCYQVTCYLTWSPCSSCAWELVDFIKAHDHLNLRIFASRLYYHWCKPQ
QDGLRLLCGSQVPVEVMGFPEFADCWENFVDHEKPLSFNPYKMLEELDKNSRAIKRRLD
RIKIPGVRAQGRYMDILCDAEV
APOBEC4:
(SEQ ID NO: 16)
MEPIYEEYLANHGTIVKPYYWLSFSLDCSNCPYHIRTGEEARVSLTEFCQIFGFPYGTTFP
QTKHLTFYELKTSSGSLVQKGHASSCTGNYIHPESMLFEMNGYLDSAIYNNDSIRHIILYS
NNSPCNEANHCCISKMYNFLITYPGITLSIYFSQLYHTEMDFPASAWNREALRSLASLWP
RVVLSPISGGIWHSVLHSFISGVSGSHVFQPILTGRALADRHNAYEINAITGVKPYFTDVLL
QTKRNPNTKAQEALESYPLNNAFPGQFFQMPSGQLQPNLPPDLRAPVVFVLVPLRDLPP
MHMGQNPNKPRNIVRHLNMPQMSFQETKDLGRLPTGRSVEIVEITEQFASSKEADEKKK
KKGKK

In some embodiments, the first domain comprises an amino acid sequence having at least 90%, 91%, 92%, 93%, 94%, 95%, 96%, 97%, 98% or 99% identity to an amino acid sequence selected from the group consisting of SEQ ID NO: 8-16, and 114. In some embodiments, the first domain comprises an amino acid sequence having at least 95%, 96%, 97%, 98% or 99% identity to an amino acid sequence selected from the group consisting of SEQ ID NO: 8-16, and 114. In some embodiments, the first domain comprises an amino acid sequence having at least 97%, 98% or 99% identity to an amino acid sequence selected from the group consisting of SEQ ID NO: 8-16, and 114. In some embodiments, the first domain comprises an amino acid sequence having 100% identity to an amino acid sequence selected from the group consisting of SEQ ID NO: 8-16, and 114.

In some embodiments, the first domain comprises an amino acid sequence having a mutation at position Y132 of human APOBEC3A amino acid sequence, e.g., SEQ ID NO: 10, or a corresponding position in a homologous or orthologous APOBEC protein. For example, the first domain comprises an amino acid sequence having a mutation at position 132 selected from the group consisting of Y312D and Y132R of APOBEC3A amino acid sequence, e.g., SEQ ID NO: 10, or a corresponding position in an APOBEC protein.

In some embodiments, the first domain comprises a catalytic domain is from m⁶A methyltransferase. For example, the first domain comprises a catalytic domain from m⁶A methyltransferase factors such as METTL3, METTL4, METTL14, and/or WTAP.

In some embodiments, the first domain comprises a catalytic domain is from an enzyme that can demethylate RNA. For example, the first domain comprises the catalytic domain from alkylation repair homolog 5 or ALKBH5.

In some embodiments, the first domain comprises an amino acid sequence having at least 80%, 81%, 82%, 83%, 84%, 85%, 86%, 87%, 88%, 89%, 90%, 91%, 92%, 93%, 94%, 95%, 96%, 97%, 98% or 99% identity to a wild-type sequence of a METTL3, METTL4, METTL14, WTAP and ALKBH5. For example, the first domain comprises an amino acid sequence having at least 80%, 81%, 82%, 83%, 84%, 85%, 86%, 87%, 88%, 89%, 90%, 91%, 92%, 93%, 94%, 95%, 96%, 97%, 98% or 99% identity to a human METTL3, METTL4, METTL14, WTAP or ALKBH5, or a homologous or orthologous thereof.

Exemplary sequences for human METTL3, METTL4, METTL14, WTAP and ALKBH5 proteins can be found in Genebank with Accession Numbers as listed: METTL3 (NP 062826.2), METTL4 isoform 1 (NP 073751.3), METTL4 isoform 2 (NP 001295330.1), METTL14 (AAH06565), WTAP isoform 1 (NP 001257460.1), WTAP isoform 2 (NP 690596.1), WTAP isoform 3 (NP 001257461.1), and ALKBH5 (NP 060228.3).

In some embodiments, the first domain comprises an amino acid sequence having at least 80%, 81%, 82%, 83%, 84%, 85%, 86%, 87%, 88%, 89%, 90%, 91%, 92%, 93%, 94%, 95%, 96%, 97%, 98% or 99% identity to an amino acid sequence selected from the group consisting of:

METTL3
(SEQ ID NO: 17)
MSDTWSSIQAHKKQLDSLRERLQRRRKQDSGHLDLRNPEAALSPTFRSDSPVPTAPTSGG
PKPSTASAVPELATDPELEKKLLHHLSDLALTLPTDAVSICLAISTPDAPATQDGVESLLQ
KFAAQELIEVKRGLLQDDAHPTLVTYADHSKLSAMMGAVAEKKGPGEVAGTVTGQKR
RAEQDSTTVAAFASSLVSGLNSSASEPAKEPAKKSRKHAASDVDLEIESLLNQQSTKEQQ
SKKVSQEILELLNTTTAKEQSIVEKFRSRGRAQVQEFCDYGTKEECMKASDADRPCRKL
HFRRIINKHTDESLGDCSFLNTCFHMDTCKYVHYEIDACMDSEAPGSKDHTPSQELALTQ
SVGGDSSADRLFPPQWICCDIRYLDVSILGKFAVVMADPPWDIHMELPYGTLTDDEMRR
LNIPVLQDDGFLFLWVTGRAMELGRECLNLWGYERVDEIIWVKTNQLQRIIRTGRTGHW
LNHGKEHCLVGVKGNPQGFNQGLDCDVIVAEVRSTSHKPDEIYGMIERLSPGTRKIELFG
RPHNVQPNWITLGNQLDGIHLLDPDVVARFKQRYPDGIISKPKNL
METTL4 isoform 1
(SEQ ID NO: 18)
MSVVHQLSAGWLLDHLSFINKINYQLHQHHEPCCRKKEFTTSVHFESLQMDSVSSSGVC
AAFIASDSSTKPENDDGGNYEMFTRKFVFRPELFDVTKPYITPAVHKECQQSNEKEDLMN
GVKKEISISIIGKKRKRCVVFNQGELDAMEYHTKIRELILDGSLQLIQEGLKSGFLYPLFEK
QDKGSKPITLPLDACSLSELCEMAKHLPSLNEMEHQTLQLVEEDTSVTEQDLFLRVVENN
SSFTKVITLMGQKYLLPPKSSFLLSDISCMQPLLNYRKTFDVIVIDPPWQNKSVKRSNRYS
YLSPLQIQQIPIPKLAAPNCLLVTWVTNRQKHLRFIKEELYPSWSVEVVAEWHWVKITNS
GEFVFPLDSPHKKPYEGLILGRVQEKTALPLRNADVNVLPIPDHKLIVSVPCTLHSHKPPL
AEVLKDYIKPDGEYLELFARNLQPGWTSWGNEVLKFQHVDYFIAVESGS
METTL4 isoform 2
(SEQ ID NO: 19)
MSVVHQLSAGWLLDHLSFINKINYQLHQHHEPCCRKKEFTTSVHFESLQMDSVSSSGVC
AAFIASDSSTKPENDDGGNYEMFTRKFVFRPELFDVTKPYITPAVHKECQQSNEKEDLMN
GVKKEISISIIGKKRKRCVVFNQGELDAMEYHTKIRELILDGSLQLIQEGLKSGFLYPLFEK
QDKGSKPITLPLDACSLSELCEMAKHLPSLNEMEHQTLQLVEEDTSVTEQDLFLRVVENN
SSFTKVITLMGQKYLLPPKSSFLLSDISCMQPLLNYRKTFDVIVIDPPWQNKSVKRSNRYS
YLSPLQIQQIPIPKLAAPNCLLVTWVTNRQKHLRFIKEELYPSWSVEVVAEWHWVKITNS
GEFVFPLDSPHKKPYEGLILGRVQEKTALPLRGFKRLHQARWGIFGVVCSKFTARLD
METTL 14
(SEQ ID NO: 20)
MDSRLQEIRERQKLRRQLLAQQLGAESADSIGAVLNSKDEQREIAETRETCRASYDTSAP
NAKRKYLDEGETDEDKMEEYKDELEMQQDEENLPYEEEIYKDSSTFLKGTQSLNPHND
YCQHFVDTGHRPQNFIRDVGLADRFEEYPKLRELIRLKDELIAKSNTPPMYLQADIEAFDI
RELTPKFDVILLEPPLEEYYRETGITANEKCWTWDDIMKLEIDEIAAPRSFIFLWCGSGEG
LDLGRVCLRKWGYRRCEDICWIKTNKNNPGKTKTLDPKAVFQRTKEHCLMGIKGTVKR
STDGDFIHANVDIDLIITEEPEIGNIEKPVEIFHIIEHFCLGRRRLHLFGRDSTIRPGWLTVGP
TLTNSNYNAETYASYFSAPNSYLTGCTEEIERLRPKSPPPKSKSDRGGGAPRGGGRGGTS
AGRGRERNRSNFRGERGGFRGGRGGAHRGGFPPR
WTAP isoform 1
(SEQ ID NO: 21)
MTNEEPLPKKVRLSETDFKVMARDELILRWKQYEAYVQALEGKYTDLNSNDVTGLRES
EEKLKQQQQESARRENILVMRLATKEQEMQECTTQIQYLKQVQQPSVAQLRSTMVDPAI
NLFFLKMKGELEQTKDKLEQAQNELSAWKFTPDSQTGKKLMAKCRMLIQENQELGRQL
SQGRIAQLEAELALQKKYSEELKSSQDELNDFIIQLDEEVEGMQSTILVLQQQLKETRQQL
AQYQQQQSQASAPSTSRTTASEPVEQSEATSKDCSRLTNGPSNGSSSRQRTSGSGFHREG
NTTEDDFPSSPGNGNKSSNSSEERTGRGGSGYVNQLSAGYESVDSPTGSENSLTHQSNDT
DSSHDPQEEKAVSGKGNRTVGSRHVQNGLDSSVNVQGSVL
WTAP isoform 2.
(SEQ ID NO: 22)
MTNEEPLPKKVRLSETDFKVMARDELILRWKQYEAYVQALEGKYTDLNSNDVTGLRES
EEKLKQQQQESARRENILVMRLATKEQEMQECTTQIQYLKQVQQPSVAQLRSTMVDPAI
NLFFLKMKGELEQTKDKLEQAQNELSAWKFTPDR
WTAP isoform 3
(SEQ ID NO: 23)
MTNEEPLPKKVRLSETDFKVMARDELILRWKQYEAYVQALEGKYTDLNSNDVTGLRES
EEKLKQQQQESARRENILVMRLATKEQEMQECTTQIQYLKQVQQPSVAQLRSTMVDPAI
NLFFLKMKGELEQTKDKLEQAQNELSAWKFTPDRGLMASDYSEEVATSEKFPF
ALKBH5
(SEQ ID NO: 24)
MAAASGYTDLREKLKSMTSRDNYKAGSREAAAAAAAAVAAAAAAAAAAEPYPVSGA
KRKYQEDSDPERSDYEEQQLQKEEEARKVKSGIRQMRLFSQDECAKIEARIDEVVSRAE
KGLYNEHTVDRAPLRNKYFFGEGYTYGAQLQKRGPGQERLYPPGDVDEIPEWVHQLVI
QKLVEHRVIPEGFVNSAVINDYQPGGCIVSHVDPIHIFERPIVSVSFFSDSALCFGCKFQFK
PIRVSEPVLSLPVRRGSVTVLSGYAADEITHCIRPQDIKERRAVIILRKTRLDAPRLETKSLS
SSVLPPSYASDRLSGNNRDPALKPKRSHRKADPDAAHRPRILEMDKEENRRSVLLPTHRR
RGSFSSENYWRKSYESSEDCSEAAGSPARKVKMRRH.

### Linkers

In some embodiments of any of the aspects, the polypeptide comprises a linker between the first domain and the second domain. The linker can be a chemical linker, a single peptide bond (e.g., linked directly to each other) or a peptide linker containing one or more amino acid residues (e.g., with an intervening amino acid or amino acid sequence between the first and second domains).

In some embodiments of any of the aspects, the linker used to link the two domains is a flexible linker. As used herein, a “flexible linker” is a linker which does not have a fixed structure (secondary or tertiary structure) in solution and is therefore free to adopt a variety of conformations. Generally, a flexible linker has a plurality of freely rotating bonds along its backbone. In contrast, a rigid linker is a linker which adopts a relatively well-defined conformation when in solution. Rigid linkers are therefore those which have a particular secondary and/or tertiary structure in solution.

In some embodiments of the various aspects described herein, the first domain and the second domain are linked via a peptide linker. The term “peptide linker” as used herein denotes a peptide with amino acid sequences, which is in some embodiments of synthetic origin. It is noted that peptide linkers may affect folding of a given fusion protein, and may also react/bind with other proteins, and these properties can be screened for by known techniques. A peptide linker can comprise 1 amino acid or more, 5 amino acids or more, 10 amino acids or more, 15 amino acids or more, 20 amino acids or more, 25 amino acids or more, 30 amino acids or more, 35 amino acids or more, 40 amino acids or more, 45 amino acids or more, 50 amino acids or more and beyond. Conversely, a peptide linker can comprise less than 50 amino acids, less than 45 amino acids, less than 40 amino acids, less than 35 amino acids, less than 30 amino acids, less than 30 amino acids, less than 25 amino acids, less than 20 amino acids, less than 15 amino acids or less than 10 amino acids.

In some embodiments of the various aspects described herein, the peptide linker comprises from about 5 amino acids to about 40 amino acids. For example, the peptide linker can comprise from about 5 amino acids to about 35 amino acids, from about 10 amino acids to 30 amino acids, or from about 10 amino acids to about 25 amino acids.

In some embodiments of the various aspects described herein, the linker comprises 10, 11, 12, 13, 14, 15, 16, 17, 18, 19, 20, 21, 22, 23, 24 or 25 amino acids. For example, the linker comprises 10, 11, 12, 13, 14, 15, 16, 17, 18, 19 or 20 amino acids. Preferably, the linker comprises 12, 13, 14, 15, 16, 17 or 18 amino acids. More preferably, the linker comprises 14, 15 or 16 amino acids. In some embodiments of the various aspects described herein, the linker comprises 15 amino acids.

Some exemplary peptide linkers include those that consist of glycine and serine residues, the so-called Gly-Ser polypeptide linkers. As used herein, the term “Gly-Ser polypeptide linker” refers to a peptide that consists of glycine and serine residues. In some embodiments of the various aspects described herein, the peptide linker comprises the amino acid sequence (GlyxSer)ₙ, where x is 2, 3, 4, 5 or 6, and n is 1, 2, 3, 4, 5, 6, 7, 8, 9 or 10 (SEQ ID NO: 25). In some embodiments of the various aspects described herein, x is 3 and n is 3, 4, 5 or 6. In some embodiments of the various aspects described herein, x is 3 and n is 4 or 5. In some embodiments of the various aspects described herein, x is 4 and n is 3, 4, 5 or 6. In some embodiments of the various aspects described herein, x is 4 and n is 4 or 5. In some embodiments of the various aspects described herein, x is 3 and n is 2. In some embodiments of the various aspects described herein, x is 3 or 4 and n is 1.

More exemplary linkers, in addition to those described herein, include a string of histidine residues, e.g., His6 (HHHHHH (SEQ ID NO: 75)); sequences made up of Ala and Pro, varying the number of Ala-Pro pairs to modulate the flexibility of the linker; and sequences made up of charged amino acid residues e.g., mixing Glu and Lys. Flexibility can be controlled by the types and numbers of residues in the linker. See, e.g., Perham, 30 Biochem. 8501 (1991); Wriggers et al., 80 Biopolymers 736 (2005).

In some embodiments, the linker comprises the amino acid sequence GGGGSLQLPPLERLTLGS (SEQ ID NO: 76), GSLQLPPLERLTLGS (SEQ ID NO: 113) or GGSGGGSSGAAAGSGG (SEQ ID NO: 77), NES sequence (SEQ ID NO: 78) in bold.

In some embodiments of the various aspects described herein, the linker can be a chemical linker. Chemical linkers can comprise a direct bond or an atom such as oxygen or sulfur, a unit such as NH, C(O), C(O)NH, SO, SO₂, SO₂NH, or a chain of atoms, such as substituted or unsubstituted C₁-C₆ alkyl, substituted or unsubstituted C₂-C₆ alkenyl, substituted or unsubstituted C₂-C₆ alkynyl, substituted or unsubstituted C₆-C₁₂ aryl, substituted or unsubstituted C₅-C₁₂ heteroaryl, substituted or unsubstituted C₅-C₁₂ heterocyclyl, substituted or unsubstituted C₃-C₁₂ cycloalkyl, where one or more methylenes can be interrupted or terminated by O, S, S(O), SO₂, NH, or C(O). The linker can be 1 amino acid or more, 5 amino acids or more, 10 amino acids or more, 15 amino acids or more, 20 amino acids or more, 25 amino acids or more, 30 amino acids or more, 35 amino acids or more, 40 amino acids or more, 45 amino acids or more, 50 amino acids or more and beyond.

In some embodiments, the polypeptide comprises a nuclear export signal. A nuclear export signal (NES) refers to a short amino acid sequence of 4 hydrophobic residues in a protein that targets it for export from the cell nucleus to the cytoplasm through the nuclear pore complex using nuclear transport. The NES is recognized and bound by exportins. The most common spacing of the hydrophobic residues to be LxxKLxxLxLX (SEQ ID NO. 79), where X is any naturally occurring amino acid. Some exemplary NES sequences include, but are not limited to, LQKKLEELELA (SEQ ID NO: 71), CIQQQLGQLTLENTL (SEQ ID NO: 80), ELALKLAGLDI (SEQ ID NO: 73), LQLPPLERLTL (SEQ ID NO: 74), ALQKKLEELELD (SEQ ID NO: 81) and TLWQFLLHLLLD (SEQ ID NO: 82). In some embodiments, the NES comprises the amino acid sequence SLQLPPLERLTL (SEQ ID NO: 78).

When present, the NES can be located anywhere in the polypeptide. For example, the NES can be at the N-terminal, C-terminal or at an internal position of the polypeptide. In some embodiments, the NES is at a position N-terminal of the first domain. In some embodiments the NES is at a position C-terminal of the first domain. In some embodiments, the NES is at a position N-terminal of the second domain. In some embodiments the NES is at a position C-terminal of the second domain.

In some embodiments, the NES is between the first and second domain. In other words, the NES is part of the linker linking the first and second domain. When the NES is between the first and second domain, there can be a linker between the first domain and the NES. Similarly, there can also be a linker between the second domain and the NES.

In some embodiments of any one of the aspects, the polypeptide can comprise an epitope or affinity tag, which can provide a convenient means for isolating or purifying the polypeptide. A number of epitope or affinity tags are known in the art. These are usually divided into 3 classes according to their size: small tags have a maximum of 12 amino acids, medium-sized ones have a maximum of 60 and large ones have more than 60. The small tags include the Arg-tag, the His-tag, the avidin biotin, or streptavidin (Strep)-tag, the Flag-tag, the T7-tag, the V5-peptide-tag and the c-Myc-tag, the medium-sized ones include the S-tag, the HAT-tag, the calmodulin-binding peptide, the chitin-binding peptide, and some cellulose-binding domains. The latter can contain up to 189 amino acids and are then regarded, like the glutathione-S-transferase (GST)- and maltose binding protein (MBP)-tag, as large affinity tags.

In some embodiments of any one of the aspects, the polypeptide comprises a Flag-tag (DYKDDDDK, SEQ ID NO: 115), a HA tag (YPYDVPDYA, SEQ ID NO: 116), ac-Myc epitope EQKLISEEDL, SEQ ID NO: 117), an AU1 tag (DTYRYI, SEQ ID NO: 118), and/or a 6-HIS tag (HHHHHH, SEQ ID NO: 75).

When present, the epitope or affinity tag can be located anywhere in the polypeptide. For example, the epitope or affinity tag can be at the N-terminal, C-terminal or at an internal position of the polypeptide. In some embodiments, the epitope or affinity tag is at a position N-terminal of the first domain. In some embodiments the epitope or affinity tag is at a position C-terminal of the first domain. In some embodiments, the epitope or affinity tag is at a position N-terminal of the second domain. In some embodiments the epitope or affinity tag is at a position C-terminal of the second domain.

In some embodiments, the epitope or affinity tag is between the first and second domain. In other words, the epitope or affinity tag is part of the linker linking the first and second domain. When the epitope or affinity tag is between the first and second domain, there can be a linker between the first domain and the epitope or affinity tag. Similarly, there can also be a linker between the second domain and the epitope or affinity tag.

In some preferred embodiments, the epitope or affinity tag is at the N-terminal of the polypeptide.

Oligonucleotide (“siRNA”)

Various aspects described herein include an oligonucleotide. In some embodiments, the oligonucleotide is double-stranded. For example, the oligonucleotide comprises a double-stranded (duplex) region. In some embodiments, the oligonucleotide comprises a first strand and second strand. For convenience, the strand having complementarity to the target RNA is also referred to as an antisense or guide strand. The other strand, i.e., the strand having complementarity to the antisense strand is also referred to as a sense or passenger strand herein.

When the oligonucleotide comprises a first and second strand, each strand can range from 12-40 nucleotides in length. For example, each strand independently can be between 14-40 nucleotides in length, 17-37 nucleotides in length, 25-37 nucleotides in length, 27-35 nucleotides in length, 17-23 nucleotides in length, 17-21 nucleotides in length, 17-19 nucleotides in length, 19-25 nucleotides in length, 19-23 nucleotides in length, 19-21 nucleotides in length, 21-25 nucleotides in length, 21-23 nucleotides in length, 25-35 nucleotides in length, 26-35 nucleotides in length, 27-34 nucleotides in length, 28-32 nucleotides in length or 29-31 nucleotides in length. Without limitations, the sense and antisense strands can be equal length or unequal length. In some embodiments, the antisense strand is longer, e.g., by 1, 2, 3, 4, or 5 nucleotides than the sense strand.

In some embodiments, the antisense strand is of length 18 to 35 nucleotides. In some embodiments, the antisense strand is 21-25, 19-25, 19-21, 21-23 nucleotides in length. In some embodiments, the antisense strand is 23, 24, 25, 26, 27, 28, 29, 30, 31 or 32 nucleotides in length. In some embodiments, the antisense strand is 23 or 31 nucleotides in length.

Similar to the antisense strand, the sense strand can be, in some embodiments, 18-35 nucleotides in length. In some embodiments, the sense strand is 21-25, 19-25, 19-21 or 21-23 nucleotides in length. In some embodiments, the antisense strand is 21, 22, 23, 24, 25, 26, 27, 28 or 29 nucleotides in length. In some preferred embodiments, the antisense strand is 21 or 29 nucleotides in length.

In some embodiments, sense strand is 21 nucleotides in length and the antisense strand is 23 nucleotides in length. In some other embodiments, sense strand is 29 nucleotides in length and the antisense strand is 31 nucleotides in length.

It is known in the art that short double-stranded oligonucleotides can induce RISC medicated cleavage of complementary target RNAs. To reduce or inhibit RISC mediated cleavage, an oligonucleotide described herein can comprise at least one modification that inhibits RISC mediated cleavage.

One way of inhibiting or reducing RISC mediated cleavage is to introduce a mismatch with the target RNA at position 8, 9, 10, 11 or 12 (counting from 5′-end) of the strand complementary to the target RNA. Accordingly, in some embodiments, the antisense strand comprises a mismatch with the target RNA at position 9, 10 or 11 (counting from 5′-end) of the antisense strand. In some particular embodiments, the antisense strand comprises a mismatch with the target RNA at position 10 (counting from 5′-end) of the antisense strand.

### A:C Mismatch

In some embodiments, the strand complementary to a target RNA, e.g., the antisense strand, comprises a mismatch with the target RNA at position 21, 22, 23, 24, 25, 26 or 27 (counting from 5′-end of said strand). For example, the antisense strand comprises a C at position 21, 22, 23, 24, 25, 26 or 27 (counting from 5′-end) and the target RNA comprises an A at the position complimentary to said C. In some particular embodiments, the antisense strand comprises a C at position 24, 25, or 26 (counting from 5′-end) and the target RNA comprises an A at the position complimentary to said C. In some particular embodiments, the antisense strand comprises a C at position 25 (counting from 5′-end) and the target RNA comprises an A at the position complimentary to said C.

In another non-limiting example, the antisense strand comprises a C at position 4, 5, 6, 7, 8, 9 or 10 (counting from 3′-end) and the target RNA comprises an A at the position complimentary to said C. In some particular embodiments, the antisense strand comprises a C at position 7 (counting from 3′-end) and the target RNA comprises an A at the position complimentary to said C in the antisense strand.

### Target Loop

Without wishing to be bound by a theory, RNA editing, e.g., C deamination with APOBEC proteins, e.g., APOBEC3A may require a loop structure in the target RNA. Thus, in some embodiments of the various aspects described herein, the oligonucleotide of the system is double-stranded and comprises a strand, e.g., the antisense strand, having a nucleotide sequence substantially complementary to a target RNA, and wherein said target RNA forms loop structure comprising a single-stranded C nucleotide when the target RNA hybridizes to said strand. It is noted that the loop structure can be from 5 to 20 nucleotides in length. In some embodiments of the various aspects described herein, the loop structure is from 10-20 nucleotides in length. For example, the loop structure is 11, 12, 13, 14, 15, 16 or 17 nucleotides in length. In some preferred embodiments, the loop structure is 13, 14 or 15 nucleotides in length. For example, the loop structure is 14 nucleotides in length.

The single stranded C nucleotide can be present at any position of the loop structure. For example, the single stranded C nucleotide can be present at position 6, 7, 8, 9, 10, 11 or 12, counting from the 5′-end of the loop structure. In some embodiments, the single stranded C nucleotide can be present at position 7, 8, 9, 10 or 11, counting from the 5′-end of the loop structure. For example, the single stranded C nucleotide can be present at position 8, 9 or 10, counting from the 5′-end of the loop structure. In some embodiments, the single stranded C nucleotide is present at position 9, counting from the 5′-end of the loop structure.

In some embodiments of the various aspects described herein, the loop structure comprises a U nucleotide 5′ to C nucleotide. For example, the loop structure comprises the dinucleotide 5′-UC-3′.

In some embodiments of the various aspects described herein, the loop structure comprises a nucleotide sequence selected from the group consisting of 5′-AAUC-3′, 5′-CAUC-3′, 5′-CCUC-3′, 5′-CUUC-3′, 5′-UAUC-3′ and 5′-CACC-3′.

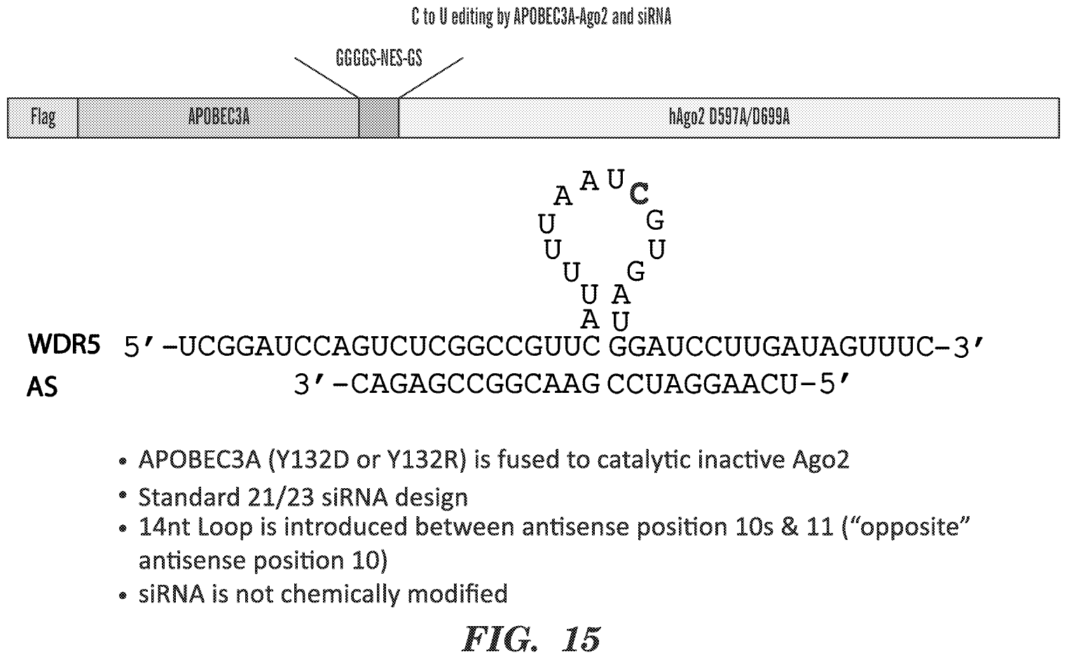

In some embodiments of the various aspects described herein, the target RNA forms the loop structure at a position opposite of position 8, 9, 10, 11, 12 or 13, counting from 5′-end, or the 3′-end, of said strand having a nucleotide sequence substantially complementary to the target RNA. “Opposite” as used in this context means that the nucleotides of the target sequence that form the loop structure begin immediately after the basepair formed between the target RNA and said strand. For example, in FIG. 15, the exemplified target RNA (for WDR5) forms a loop structure “opposite” position 10 of the exemplified antisense strand, counting from 5′-end of the antisense strand.

In some embodiments of the various aspects described herein, the target RNA forms the loop structure at a position opposite of position 8, 9, 10, 11, 12 or 13, counting from the 5′-end of said strand having a nucleotide sequence substantially complementary to the target RNA. For example, the target RNA forms a hairpin structure at a position opposite of position 9, 10, 11, or 12, counting from the 5′-end of said strand having a nucleotide sequence substantially complementary to the target nucleic acid. In some preferred embodiments, the target nucleic forms a hairpin structure at a position opposite of position 10 or 11, counting from the 5′-end of said strand having a nucleotide sequence substantially complementary to the target RNA.

In some preferred embodiments, the target nucleic forms a hairpin structure at a position opposite of position 9, counting from the 5′-end of said strand having a nucleotide sequence substantially complementary to the target RNA. In some preferred embodiments, the target nucleic forms a hairpin structure at a position opposite of position 10, counting from the 5′-end of said strand having a nucleotide sequence substantially complementary to the target RNA. In some preferred embodiments, the target nucleic forms a hairpin structure at a position opposite of position 11, counting from the 5′-end of said strand having a nucleotide sequence substantially complementary to the target RNA.

In other examples, the target RNA forms a hairpin structure at a position opposite of position 9, 10, 11, or 12, counting from the 3′-end of said strand having a nucleotide sequence substantially complementary to the target nucleic acid. In some preferred embodiments, the target nucleic forms a hairpin structure at a position opposite of position 10 or 11, counting from the 3′-end of said strand having a nucleotide sequence substantially complementary to the target RNA.

In some preferred embodiments, the target nucleic forms a hairpin structure at a position opposite of position 9, counting from the 3′-end of said strand having a nucleotide sequence substantially complementary to the target RNA. In some preferred embodiments, the target nucleic forms a hairpin structure at a position opposite of position 10, counting from the 3′-end of said strand having a nucleotide sequence substantially complementary to the target RNA. In some preferred embodiments, the target nucleic forms a hairpin structure at a position opposite of position 11, counting from the 3′-end of said strand having a nucleotide sequence substantially complementary to the target RNA.

In some embodiments of any one of the aspects, the loop structure is in the form of a hairpin comprising a single-stranded region and a double-stranded region (stem). In some embodiments of the various aspects described herein, the hairpin comprises a single-stranded region of 3-15 nucleotides in length. For example, the single-stranded region of the hairpin is 5, 6, 7, 8, 9, 10, 11, 12 or 13 nucleotides in length. In some embodiments, the single-stranded region of the hairpin is 7, 8, 9, 10, 11, 12 or 13 nucleotides in length. For example, the single-stranded region of the hairpin is 8, 9, 10, 11 or nucleotides in length. In some preferred embodiments, the single-stranded region of the hairpin is 10 nucleotides in length.

In some embodiments of the various aspects described herein, the stem of the hairpin comprises a double-stranded region of 2-10 basepairs in length. For example, the stem of the hairpin comprises a double-stranded region of 2, 3, 4, 5, 6, 7, 8 or 9 basepairs in length. In some embodiments, the stem of the hairpin comprises a double-stranded region of 2, 3, 4, 5, 6 or 7 basepairs in length. For example, the stem of the hairpin comprises a double-stranded region of 2, 3, 4, 5, 6 or 7 basepairs in length. In some embodiments, the hairpin comprises a double-stranded region of 2 or 3 basepairs in length.

In some embodiments of any one of the aspects described herein, target RNA sequence forming the single-stranded region of the hairpin structure is flanked by palindromic sequences. For example, the sequence forming single-stranded region of the hairpin is flanked by palindromic sequences and wherein the palindromic sequences hybridize to form the double-stranded region of the hairpin when the target RNA is hybridized with the strand having a nucleotide sequence substantially complementary to the target RNA.

As described herein, the single-stranded region of the hairpin comprises a C nucleotide. Said C nucleotide can be present anywhere in the single-stranded region. For example, the C nucleotide can be at position 1, 2, 3, 4, 5, 6, 7, 8, 9 or 10, counting from either end, of the single-stranded region.

In some embodiments of any one of the aspects described herein, the single-stranded region of the hairpin structure comprises a nucleotide sequence 5′-UC-3′, where the C is the editing location. For example, the single-stranded region of the hairpin structure comprises a nucleotide sequence selected from the group consisting of 5′-AAUC-3′, 5′-CAUC-3′, 5′-CCUC-3′, 5′-CUUC-3′, 5′-UAUC-3′ and 5′-CACC-3′, where the 3′-end C is the editing location.

In some embodiments, the antisense strand is phosphorylated at the 5′-end.

The double-stranded oligonucleotide has a double-stranded or duplex region. Generally, the duplex region (double-stranded region) is 12-40 nucleotide base pairs in length. For example, the dsRNA has a duplex region of 15-35 nucleotide pairs in length. In some embodiments, the double-stranded oligonucleotide has a duplex region of 18, 19, 20, 21, 22, 22, 23, 24, 25, 26, 27, 28, 29, 30 or 31 nucleotide base pairs in length. In some particular embodiments, the double-stranded oligonucleotide has a duplex region of 19, 20, 21 or 22 nucleotide base pairs in length. In some other particular embodiments, the double-stranded oligonucleotide has a duplex region of 28, 29, 30 or 31 nucleotide base pairs in length.

In some embodiments, the double-stranded oligonucleotide comprises one or more overhang regions (i.e., single-stranded region) and/or capping groups of oligonucleotides at the 3′-end, or 5′-end, or both ends of a strand. Without limitations, the overhang can be 1-10 nucleotides in length, 1-6 nucleotides in length, 1-5 nucleotides in length, 1-4 nucleotides in length, 1-3 nucleotides in length, 2-6 nucleotides in length, 2-5 nucleotides in length 2-4 nucleotides in length, 2-3 nucleotides in length, or 1-2 nucleotides in length. The overhangs can be the result of one strand being longer than the other, or the result of two strands of the same length being staggered. The overhang can form a mismatch with the sequence being targeted or it can be complementary to the sequence being targeted or can be other sequence. The first and second strands can also be joined, e.g., by additional bases to form a hairpin, or by other non-base linkers. Without limitations the overhang can be present at the 3′-end of the sense strand, antisense strand or both strands.

In some embodiments, the double-stranded oligonucleotide comprises a single overhang. For example, the double-stranded oligonucleotide has a single overhang and the overhang is at least two, three, four, five, six, seven, eight, nine, or ten nucleotides in length. In some embodiments, the overhang is present at the 3′-end of the antisense strand. In some particular embodiments, the double-stranded oligonucleotide comprises a two nucleotide overhang at the 3′-end of the antisense strand.

The double-stranded oligonucleotide can also have a blunt end. For example, one end of the double-stranded oligonucleotide is a blunt end and the other end has an overhang. Without limitations, the blunt end can be located at the 5′-end of the antisense strand (or the 3′-end of the sense strand) or vice versa. Generally, the antisense strand of the double-stranded oligonucleotide has a nucleotide overhang at the 3′-end, and the 5′-end is blunt. In some embodiments, the double-stranded oligonucleotide has a 2 nucleotide overhang on the 3′-end of the antisense strand and a blunt end at the 5′-end of the antisense strand.

In some other embodiments, the double-stranded oligonucleotide has two blunt ends, i.e., at both ends of the double-stranded oligonucleotide.

The nucleotides in the overhang region can each independently be a modified or unmodified nucleotide including, but not limited to 2′-sugar modified, such as, 2′-Fluoro, 2′-O-methyl, thymidine (T), 2′-O-methoxyethyl-5-methyluridine, 2′-O-methoxyethyladenosine, 2′-O-methoxyethyl-5-methylcytidine, GNA, SNA, hGNA, hhGNA, mGNA, TNA, h'GNA, and any combinations thereof. For example, TT (or UU) can be an overhang sequence for either end on either strand. The 5′- or 3′—overhangs at the sense strand, antisense strand or both strands can be phosphorylated. In some embodiments, the overhang region contains two nucleotides having a phosphorothioate internucleotide linkage between the two nucleotides, where the two nucleotides in the overhang region can be the same or different.

### Modifications to the Oligonucleotides

In some embodiments of any one of the aspects, the oligonucleotide can comprise one or more nucleic acid modifications. For example, the oligonucleotide can comprise at least one, e.g., e.g., two, three, four, five, six, seven, eight, nine, ten, eleven, twelve, thirteen, fourteen, fifteen or more nucleic acid modifications. It is noted that when two are more modifications are present, they can be same, different or some combination of same and different. Further, when the oligonucleotide is double-stranded, the modifications all can be present in one strand. In some embodiments, both strands comprise at least one nucleic acid modification. When both strands comprise at least one modification, the modifications can be same, different or some combination of same and different.

In some embodiments, the oligonucleotide can comprise 2′-fluoro nucleotides, i.e., 2′-fluoro modifications. For example, the oligonucleotide can comprise at least four, e.g., five, six, seven, eight, nine, ten, eleven, twelve, thirteen, fourteen, fifteen or more 2′-fluoro nucleotides. When the oligonucleotide is double-stranded, the 2′-fluoro nucleotides all can be present in one strand.

In some embodiments, both the sense and the antisense strands comprise at least two 2′-fluoro nucleotides. The 2′-fluoro modification can occur on any nucleotide of the sense strand or antisense strand. For instance, the 2′-fluoro modification can occur on every nucleotide on the sense strand and/or antisense strand; each 2′-fluoro modification can occur in an alternating pattern on the sense strand or antisense strand; or the sense strand and antisense strand both comprise 2′-fluoro modifications in an alternating pattern. The alternating pattern of the 2′-fluoro modifications on the sense strand can be the same or different from the antisense strand, and the alternating pattern of the 2′-fluoro modifications on the sense strand can have a shift relative to the alternating pattern of the 2′-fluoro modifications on the antisense strand.

The antisense strand can comprise at least two (e.g., two, three, four, five, six, seven, eight, nine, ten or more) 2′-fluoro nucleotides. In some embodiments, the antisense strand comprises two, three, four, five or six 2′-fluoro nucleotides. Without limitations, a 2′-fluoro modification in the antisense strand can be present at any position. In some embodiments, the antisense strand comprises at least three 2′-fluoro nucleotides. For example, the antisense strand comprises 2′-fluoro nucleotides at least at positions 2, 14 and 16 from the 5′-end. In some other embodiments, the antisense comprises at least four 2′-fluoro nucleotides. For example, the antisense comprises 2′-fluoro nucleotides at least at positions 2, 6, 14 and 16 from the 5′-end. In some further embodiments, the antisense strand comprises at least five 2′-fluoro nucleotides. For example, the antisense strand comprises 2′-fluoro nucleotides at least at positions 2, 6, 9, 14 and 16 from the 5′-end. In still some further embodiments, the antisense strand comprises at least six 2′-fluoro nucleotides. For example, the antisense strand comprises 2′-fluoro nucleotides at least at positions 2, 6, 8, 9, 14 and 16 from the 5′-end.

The sense strand can comprise at least two (e.g., two, three, four, five, six, seven, eight, nine, ten or more) 2′-fluoro nucleotides. In some embodiments, the sense strand comprises two, three, four, or five 2′-fluoro nucleotides. For example, the sense strand comprises three or four 2′-fluoro nucleotides. Without limitations, a 2′-fluoro modification in the sense strand can be present at any positions. In some embodiments, the sense strand comprises at least three 2′-fluoro nucleotides. For example, the sense comprises 2′-fluoro nucleotides at least at positions 7, 10 and 11 from the 5′-end. In some other embodiments, the sense strand comprises at least four 2′-fluoro nucleotides. For example, the sense comprises 2′-fluoro nucleotides at least at positions 7, 9, 10 and 11 from the 5′-end.

In some embodiments, the sense strand comprises 2′-fluoro nucleotides at positions opposite or complimentary to positions 11, 12 and 15 of the antisense strand, counting from the 5′-end of the antisense strand. In some other embodiments, the sense strand comprises 2′-fluoro nucleotides at positions opposite or complimentary to positions 11, 12, 13, and 15 of the antisense strand, counting from the 5′-end of the antisense strand. In some embodiments, the sense strand comprises a block of two, three or four 2′-fluoro nucleotides.

In some embodiments, the sense strand comprises 2′-fluoro nucleotides at least at positions 7, 9, and 11 from the 5′-end, and the antisense strand comprises 2′-fluoro nucleotides at least at positions 2, 14 and 16 from the 5′-end. In some other embodiments, the sense strand comprises 2′-fluoro nucleotides at least at positions 7, 9, and 11 from the 5′-end, and the antisense strand comprises 2′-fluoro nucleotides at least at positions 2, 6, 9, 14 and 16 from the 5′-end. In yet some other embodiments, the sense strand comprises 2′-fluoro nucleotides at least at positions 7, 9, and 11 from the 5′-end, and the antisense strand comprises 2′-fluoro nucleotides at least at positions 2, 6, 8, 9, 14 and 16 from the 5′-end.

In some embodiments, the sense strand comprises 2′-fluoro nucleotides at least at positions 7, 9, 10, and 11 from the 5′-end, and the antisense strand comprises 2′-fluoro nucleotides at least at positions 2, 14 and 16 from the 5′-end. In some other embodiments, the sense strand comprises 2′-fluoro nucleotides at least at positions 7, 9, 10, and 11 from the 5′-end, and the antisense strand comprises 2′-fluoro nucleotides at least at positions 2, 6, 9, 14 and 16 from the 5′-end. In yet some other embodiments, the sense strand comprises 2′-fluoro nucleotides at least at positions 7, 9, 10, and 11 from the 5′-end, and the antisense strand comprises 2′-fluoro nucleotides at least at positions 2, 6, 8, 9, 14 and 16 from the 5′-end.

In some embodiments, the antisense strand does not comprise a 2′-fluoro nucleotide at positions 3-9, counting from 5′-end.

In some embodiments, the oligonucleotide can comprise at least one, e.g., one, two, three, four, five, six, seven, eight, nine, ten, eleven, twelve, thirteen, fourteen, fifteen, sixteen, seventeen, eighteen, nineteen, twenty or more 2′-OMe nucleotides.

When the oligonucleotide is double-stranded, the 2′-OMe nucleotides all can be present in one strand. In some embodiments, both the sense and the antisense strands comprise at least one 2′-OMe nucleotide. The 2′-OMe modification can occur on any nucleotide of the sense strand or antisense strand. For instance, the 2′-OMe modification can occur on every nucleotide on the sense strand and/or antisense strand; each thermally stabilizing modification can occur in an alternating pattern on the sense strand or antisense strand; or the sense strand and antisense strand both comprise 2′-OMe modifications in an alternating pattern. The alternating pattern of the thermally stabilizing modifications on the sense strand can be the same or different from the antisense strand, and the alternating pattern of the thermally stabilizing modifications on the sense strand can have a shift relative to the alternating pattern of the 2′-OMe modifications on the antisense strand.

The antisense strand can comprise at least one, e.g., two, three, four, five, six, seven, eight, nine, ten, eleven, twelve, thirteen, fourteen, fifteen, sixteen, seventeen or more 2′-OMe modifications. Without limitations, a thermally stabilizing modification in the antisense strand can be present at any position. In some embodiments, the antisense strand comprises at least three thermally stabilizing modifications.

For example, the antisense strand does not comprise 2′-OMe modifications at least at positions 2, 14 and 16 from the 5′-end. In some other embodiments, the antisense does not comprise 2′-OMe modifications at least at positions 2, 6, 14 and 16 from the 5′-end. In some further embodiments, the antisense strand does not comprise 2′-OMe modifications at least at positions 2, 6, 9, 14 and 16 from the 5′-end. In still some further embodiments, the antisense strand does not comprise 2′-OMe modifications at least at positions 2, 6, 8, 9, 14 and 16 from the 5′-end.

The sense strand can comprise at least one, e.g., two, three, four, five, six, seven, eight, nine, ten, eleven, twelve, thirteen, fourteen, fifteen, sixteen or more 2′-OMe modifications. Without limitations, a 2′-OMe modification in the sense strand can be present at any positions. In some embodiments, the sense does not comprise 2′-OMe modifications at least at positions 7, 10 and 11 from the 5′-end. In some other embodiments, the sense does not comprise 2′-OMe modifications at least at positions 7, 9, 10 and 11 from the 5′-end.

In some embodiments, the oligonucleotide can comprise locked nucleic acid (LNA). For example, the oligonucleotide can comprise at least one, e.g., one, two, three, four, five, six, seven, eight, nine, ten, eleven, twelve, thirteen, fourteen, fifteen, sixteen, seventeen, eighteen, nineteen, twenty or more LNA modifications.

When the oligonucleotide is double-stranded, the LNA nucleotides all can be present in one strand. In some embodiments, both the sense and the antisense strands comprise at least LNA modifications. The LNA modification can occur on any nucleotide of the sense strand or antisense strand. For instance, the LNA modification can occur on every nucleotide on the sense strand and/or antisense strand; each LNA modification can occur in an alternating pattern on the sense strand or antisense strand; or the sense strand and antisense strand both comprise LNA modifications in an alternating pattern. The alternating pattern of the LNA modifications on the sense strand can be the same or different from the antisense strand, and the alternating pattern of the LNA modifications on the sense strand can have a shift relative to the alternating pattern of the 2′-fluoro modifications on the antisense strand.

The antisense strand can comprise at least one, e.g., two, three, four, five, six, seven, eight, nine, ten or more LNA modifications. Without limitations, a LNA modification in the antisense strand can be present at any position.

The sense strand can comprise at least one, e.g., two, three, four, five, six, seven, eight, nine, ten or more LNA modifications. Without limitations, a LNA modification in the sense strand can be present at any position. In some embodiments, the sense strand comprises at least one, e.g., two, three, four, five, six, seven, eight, nine, ten or more LNA modifications and the antisense strand does not comprise a 2′-fluoro nucleotide at positions 3-9, counting from 5′-end.

The oligonucleotide can comprise bridged nucleic acid (BNA). For example, the oligonucleotide can comprise at least one, e.g., one, two, three, four, five, six, seven, eight, nine, ten, eleven, twelve, thirteen, fourteen, fifteen, sixteen, seventeen, eighteen, nineteen, twenty or more BNA modifications. Without limitations, the BNA nucleotides all can be present in one strand. In some embodiments, both the sense and the antisense strands comprise at least BNA modifications. The BNA modification can occur on any nucleotide of the sense strand or antisense strand. For instance, the BNA modification can occur on every nucleotide on the sense strand and/or antisense strand; each BNA modification can occur in an alternating pattern on the sense strand or antisense strand; or the sense strand and antisense strand both comprise BNA modifications in an alternating pattern. The alternating pattern of the BNA modifications on the sense strand can be the same or different from the antisense strand, and the alternating pattern of the BNA modifications on the sense strand can have a shift relative to the alternating pattern of the 2′-fluoro modifications on the antisense strand.

The antisense strand can comprise at least one, e.g., two, three, four, five, six, seven, eight, nine, ten or more BNA modifications. Without limitations, a BNA modification in the antisense strand can be present at any position.

The sense strand can comprise at least one, e.g., two, three, four, five, six, seven, eight, nine, ten or more BNA modifications. Without limitations, a BNA modification in the sense strand can be present at any position. In some embodiments, the sense strand comprises at least one, e.g., two, three, four, five, six, seven, eight, nine, ten or more BNA modifications and the antisense strand does not comprise a 2′-fluoro nucleotide at positions 3-9, counting from 5′-end.

The oligonucleotide can comprise cyclohexene nucleic acid (CeNA). For example, the oligonucleotide can comprise at least one, e.g., one, two, three, four, five, six, seven, eight, nine, ten, eleven, twelve, thirteen, fourteen, fifteen, sixteen, seventeen, eighteen, nineteen, twenty or more CeNA modifications. Without limitations, the CeNA nucleotides all can be present in one strand. In some embodiments, both the sense and the antisense strands comprise at least CeNA modifications. The CeNA modification can occur on any nucleotide of the sense strand or antisense strand. For instance, the CeNA modification can occur on every nucleotide on the sense strand and/or antisense strand; each CeNA modification can occur in an alternating pattern on the sense strand or antisense strand; or the sense strand and antisense strand both comprise CeNA modifications in an alternating pattern. The alternating pattern of the CeNA modifications on the sense strand can be the same or different from the antisense strand, and the alternating pattern of the CeNA modifications on the sense strand can have a shift relative to the alternating pattern of the 2′-fluoro modifications on the antisense strand.

The antisense strand can comprise at least one, e.g., two, three, four, five, six, seven, eight, nine, ten or more CeNA modifications. Without limitations, a CeNA modification in the antisense strand can be present at any position.

The sense strand can comprise at least one, e.g., two, three, four, five, six, seven, eight, nine, ten or more CeNA modifications. Without limitations, a CeNA modification in the sense strand can be present at any position. In some embodiments, the sense strand comprises at least one, e.g., two, three, four, five, six, seven, eight, nine, ten or more CeNA modifications and the antisense strand does not comprise a 2′-fluoro nucleotide at positions 3-9, counting from 5′-end.

The oligonucleotide can comprise thermally stabilizing modifications. For example, the oligonucleotide can comprise at least four, e.g., five, six, seven, eight, nine, ten, eleven, twelve, thirteen, fourteen, fifteen or more thermally stabilizing modifications. When the oligonucleotide is double-stranded, the thermally stabilizing modifications all can be present in one strand. In some embodiments, both the sense and the antisense strands comprise at least one, e.g., two, three, four or more thermally stabilizing modifications. The thermally stabilizing modification can occur on any nucleotide of the sense strand or antisense strand. For instance, the thermally stabilizing modification can occur on every nucleotide on the sense strand and/or antisense strand; each thermally stabilizing modification can occur in an alternating pattern on the sense strand or antisense strand; or the sense strand and antisense strand both comprise thermally stabilizing modifications in an alternating pattern. The alternating pattern of the thermally stabilizing modifications on the sense strand can be the same or different from the antisense strand, and the alternating pattern of the thermally stabilizing modifications on the sense strand can have a shift relative to the alternating pattern of the thermally stabilizing modifications on the antisense strand.

The antisense strand can comprise at least one, e.g., two, three, four, five, six, seven, eight, nine, ten or more thermally stabilizing modifications. In some embodiments, the antisense strand comprises two, three, four, five or six thermally stabilizing modifications. Without limitations, a thermally stabilizing modification in the antisense strand can be present at any position. In some embodiments, the antisense strand comprises at least three thermally stabilizing modifications. For example, the antisense strand comprises thermally stabilizing modifications at least at positions 2, 14 and 16 from the 5′-end. In some other embodiments, the antisense comprises at least four thermally stabilizing modifications. For example, the antisense comprises thermally stabilizing modifications at least at positions 2, 6, 14 and 16 from the 5′-end. In some further embodiments, the antisense strand comprises at least five thermally stabilizing modifications. For example, the antisense strand comprises thermally stabilizing modifications at least at positions 2, 6, 9, 14 and 16 from the 5′-end. In still some further embodiments, the antisense strand comprises at least six thermally stabilizing modifications. For example, the antisense strand comprises thermally stabilizing modifications at least at positions 2, 6, 8, 9, 14 and 16 from the 5′-end.

The sense strand can comprise at least one, e.g., two, three, four, five, six, seven, eight, nine, ten or more thermally stabilizing modifications. In some embodiments, the sense strand comprises two, three, four, or five thermally stabilizing modifications. For example, the sense strand comprises three or four thermally stabilizing modifications. Without limitations, a thermally stabilizing modification in the sense strand can be present at any positions. In some embodiments, the sense strand comprises at least three thermally stabilizing modifications. For example, the sense comprises thermally stabilizing modification at least at positions 7, 10 and 11 from the 5′-end. In some other embodiments, the sense strand comprises at least four thermally stabilizing modifications. For example, the sense comprises thermally stabilizing modification at least at positions 7, 9, 10 and 11 from the 5′-end.

In some embodiments, the sense strand comprises thermally stabilizing modifications at positions opposite or complimentary to positions 11, 12 and 15 of the antisense strand, counting from the 5′-end of the antisense strand. In some other embodiments, the sense strand comprises thermally stabilizing modifications at positions opposite or complimentary to positions 11, 12, 13 and 15 of the antisense strand, counting from the 5′-end of the antisense strand. In some embodiments, the sense strand comprises a block of two, three or four thermally stabilizing modification.

In some embodiments, the sense strand comprises thermally stabilizing modifications at least at positions 7, 9, and 11 from the 5′-end, and the antisense strand comprises thermally stabilizing modifications at least at positions 2, 14 and 16 from the 5′-end. In some other embodiments, the sense strand comprises thermally stabilizing modifications at least at positions 7, 9, and 11 from the 5′-end, and the antisense strand comprises thermally stabilizing modifications at least at positions 2, 6, 9, 14 and 16 from the 5′-end. In yet some other embodiments, the sense strand comprises thermally stabilizing modifications at least at positions 7, 9, and 11 from the 5′-end, and the antisense strand comprises thermally stabilizing modifications at least at positions 2, 6, 8, 9, 14 and 16 from the 5′-end.

In some embodiments, the sense strand comprises thermally stabilizing modifications at least at positions 7, 9, 10, and 11 from the 5′-end, and the antisense strand comprises thermally stabilizing modifications at least at positions 2, 14 and 16 from the 5′-end. In some other embodiments, the sense strand comprises thermally stabilizing modifications at least at positions 7, 9, 10, and 11 from the 5′-end, and the antisense strand comprises thermally stabilizing modifications at least at positions 2, 6, 9, 14 and 16 from the 5′-end. In yet some other embodiments, the sense strand comprises thermally stabilizing modifications at least at positions 7, 9, 10, and 11 from the 5′-end, and the antisense strand comprises thermally stabilizing modifications at least at positions 2, 6, 8, 9, 14 and 16 from the 5′-end.

In some embodiments, the sense strand does not comprise a thermally stabilizing modification in position opposite or complimentary to the thermally destabilizing modification of the duplex in the antisense strand.

Exemplary thermally stabilizing modifications include, but are not limited to, 2′-fluoro modifications and locked nucleic acid (LNA).

The oligonucleotide can comprise at least one, e.g., two, three, four, five, six, seven, eight, nine, ten or more phosphorothioate or methylphosphonate internucleotide linkage. The phosphorothioate or methylphosphonate internucleotide linkage modification can occur on any nucleotide of the oligonucleotide. When the oligonucleotide is double-stranded, the phosphorothioate or methylphosphonate internucleotide linkage modification can occur in the sense strand or antisense strand or both in any position of the strand. For instance, the internucleotide linkage modification can occur on every nucleotide on the sense strand and/or antisense strand; each internucleotide linkage modification can occur in an alternating pattern on the sense strand or antisense strand; or the sense strand or antisense strand comprises both internucleotide linkage modifications in an alternating pattern. The alternating pattern of the internucleotide linkage modification on the sense strand can be the same or different from the antisense strand, and the alternating pattern of the internucleotide linkage modification on the sense strand can have a shift relative to the alternating pattern of the internucleotide linkage modification on the antisense strand.

In some embodiments, the double-stranded oligonucleotide comprises the phosphorothioate or methylphosphonate internucleotide linkage modification in the overhang region. For example, the overhang region comprises two nucleotides having a phosphorothioate or methylphosphonate internucleotide linkage between the two nucleotides. Internucleotide linkage modifications also may be made to link the overhang nucleotides with the terminal paired nucleotides within duplex region. For example, at least 2, 3, 4, or all the overhang nucleotides can be linked through phosphorothioate or methylphosphonate internucleotide linkage, and optionally, there may be additional phosphorothioate or methylphosphonate internucleotide linkages linking the overhang nucleotide with a paired nucleotide that is next to the overhang nucleotide. For instance, there may be at least two phosphorothioate internucleotide linkages between the terminal three nucleotides, in which two of the three nucleotides are overhang nucleotides, and the third is a paired nucleotide next to the overhang nucleotide. Preferably, these terminal three nucleotides can be at the 3′-end of the antisense strand.

In some embodiments, the sense strand comprises 1-10 blocks of two to ten phosphorothioate or methylphosphonate internucleotide linkages separated by 1, 2, 3, 4, 5, 6, 7, 8, 9, 10, 11, 12, 13, 14, 15 or 16 phosphate internucleotide linkages, wherein one of the phosphorothioate or methylphosphonate internucleotide linkages is placed at any position in the oligonucleotide sequence and the said sense strand is paired with an antisense strand comprising any combination of phosphorothioate, methylphosphonate and phosphate internucleotide linkages or an antisense strand comprising either phosphorothioate or methylphosphonate or phosphate linkage.

In some embodiments, the antisense strand comprises two blocks of two phosphorothioate or methylphosphonate internucleotide linkages separated by 1, 2, 3, 4, 5, 6, 7, 8, 9, 10, 11, 12, 13, 14, 15, 16, 17, or 18 phosphate internucleotide linkages, wherein one of the phosphorothioate or methylphosphonate internucleotide linkages is placed at any position in the oligonucleotide sequence and the said antisense strand is paired with a sense strand comprising any combination of phosphorothioate, methylphosphonate and phosphate internucleotide linkages or an antisense strand comprising either phosphorothioate or methylphosphonate or phosphate linkage.

In some embodiments, the antisense strand comprises two blocks of three phosphorothioate or methylphosphonate internucleotide linkages separated by 1, 2, 3, 4, 5, 6, 7, 8, 9, 10, 11, 12, 13, 14, 15 or 16 phosphate internucleotide linkages, wherein one of the phosphorothioate or methylphosphonate internucleotide linkages is placed at any position in the oligonucleotide sequence and the said antisense strand is paired with a sense strand comprising any combination of phosphorothioate, methylphosphonate and phosphate internucleotide linkages or an antisense strand comprising either phosphorothioate or methylphosphonate or phosphate linkage.

In some embodiments, the antisense strand comprises two blocks of four phosphorothioate or methylphosphonate internucleotide linkages separated by 1, 2, 3, 4, 5, 6, 7, 8, 9, 10, 11, 12, 13 or 14 phosphate internucleotide linkages, wherein one of the phosphorothioate or methylphosphonate internucleotide linkages is placed at any position in the oligonucleotide sequence and the said antisense strand is paired with a sense strand comprising any combination of phosphorothioate, methylphosphonate and phosphate internucleotide linkages or an antisense strand comprising either phosphorothioate or methylphosphonate or phosphate linkage.

In some embodiments, the antisense strand comprises two blocks of five phosphorothioate or methylphosphonate internucleotide linkages separated by 1, 2, 3, 4, 5, 6, 7, 8, 9, 10, 11 or 12 phosphate internucleotide linkages, wherein one of the phosphorothioate or methylphosphonate internucleotide linkages is placed at any position in the oligonucleotide sequence and the said antisense strand is paired with a sense strand comprising any combination of phosphorothioate, methylphosphonate and phosphate internucleotide linkages or an antisense strand comprising either phosphorothioate or methylphosphonate or phosphate linkage.

In some embodiments, the antisense strand comprises two blocks of six phosphorothioate or methylphosphonate internucleotide linkages separated by 1, 2, 3, 4, 5, 6, 7, 8, 9 or 10 phosphate internucleotide linkages, wherein one of the phosphorothioate or methylphosphonate internucleotide linkages is placed at any position in the oligonucleotide sequence and the said antisense strand is paired with a sense strand comprising any combination of phosphorothioate, methylphosphonate and phosphate internucleotide linkages or an antisense strand comprising either phosphorothioate or methylphosphonate or phosphate linkage.

In some embodiments, the antisense strand comprises two blocks of seven phosphorothioate or methylphosphonate internucleotide linkages separated by 1, 2, 3, 4, 5, 6, 7 or 8 phosphate internucleotide linkages, wherein one of the phosphorothioate or methylphosphonate internucleotide linkages is placed at any position in the oligonucleotide sequence and the said antisense strand is paired with a sense strand comprising any combination of phosphorothioate, methylphosphonate and phosphate internucleotide linkages or an antisense strand comprising either phosphorothioate or methylphosphonate or phosphate linkage.

In some embodiments, the antisense strand comprises two blocks of eight phosphorothioate or methylphosphonate internucleotide linkages separated by 1, 2, 3, 4, 5 or 6 phosphate internucleotide linkages, wherein one of the phosphorothioate or methylphosphonate internucleotide linkages is placed at any position in the oligonucleotide sequence and the said antisense strand is paired with a sense strand comprising any combination of phosphorothioate, methylphosphonate and phosphate internucleotide linkages or an antisense strand comprising either phosphorothioate or methylphosphonate or phosphate linkage.

In some embodiments, the antisense strand comprises two blocks of nine phosphorothioate or methylphosphonate internucleotide linkages separated by 1, 2, 3 or 4 phosphate internucleotide linkages, wherein one of the phosphorothioate or methylphosphonate internucleotide linkages is placed at any position in the oligonucleotide sequence and the said antisense strand is paired with a sense strand comprising any combination of phosphorothioate, methylphosphonate and phosphate internucleotide linkages or an antisense strand comprising either phosphorothioate or methylphosphonate or phosphate linkage.

In some embodiments, the double-stranded oligonucleotide comprises one or more phosphorothioate or methylphosphonate internucleotide linkage modification within 1-10 of the termini position(s) of the sense and/or antisense strand. For example, at least 2, 3, 4, 5, 6, 7, 8, 9 or 10 nucleotides may be linked through phosphorothioate or methylphosphonate internucleotide linkage at one end or both ends of the sense and/or antisense strand.

In some embodiments, the double-stranded oligonucleotide comprises one or more phosphorothioate or methylphosphonate internucleotide linkage modification within 1-10 of the internal region of the duplex of each of the sense and/or antisense strand. For example, at least 2, 3, 4, 5, 6, 7, 8, 9 or 10 nucleotides may be linked through phosphorothioate methylphosphonate internucleotide linkage at position 8-16 of the duplex region counting from the 5′-end of the sense strand; the oligonucleotide can optionally further comprise one or more phosphorothioate or methylphosphonate internucleotide linkage modification within 1-10 of the termini position(s).

In some embodiments, the double-stranded oligonucleotide comprises one to five phosphorothioate or methylphosphonate internucleotide linkage modification(s) within position 1-5 (counting from the 5′-end) and one to five phosphorothioate or methylphosphonate internucleotide linkage modification(s) within position 1-5 (counting from the 3′-end) of the sense strand, and one to five phosphorothioate or methylphosphonate internucleotide linkage modification at positions 1 and 2 (counting from the 5′-end) and one to five within positions 1-5 (counting from the 3′-end) of the antisense strand.

In some embodiments, the oligonucleotide comprises one phosphorothioate internucleotide linkage modification within position 1-5 (counting from the 5′-end) and one phosphorothioate or methylphosphonate internucleotide linkage modification within position 1-5 (counting from the 3′-end) of the sense strand, and one phosphorothioate internucleotide linkage modification at positions 1 and 2 (counting from the 5′-end) and two phosphorothioate or methylphosphonate internucleotide linkage modifications within positions 1-5 (counting from the 3′-end) of the antisense strand.

In some embodiments, the oligonucleotide comprises two phosphorothioate internucleotide linkage modifications within position 1-5 (counting from the 5′-end) and one phosphorothioate internucleotide linkage modification within position 1-5 (counting from the 3′-end) of the sense strand, and one phosphorothioate internucleotide linkage modification at positions 1 and 2 (counting from the 5′-end) and two phosphorothioate internucleotide linkage modifications within positions 18-23 (counting from the 3′-end) of the antisense strand.

In some embodiments, the oligonucleotide comprises two phosphorothioate internucleotide linkage modifications within position 1-5 (counting from the 5′-end) and two phosphorothioate internucleotide linkage modifications within position 1-5 (counting from the 3′-end) of the sense strand, and one phosphorothioate internucleotide linkage modification at positions 1 and 2 (counting from the 5′-end) and two phosphorothioate internucleotide linkage modifications within positions 1-5 (counting from the 3′-end) of the antisense strand.

In some embodiments, the oligonucleotide comprises two phosphorothioate internucleotide linkage modifications within position 1-5 (counting from the 5′-end) and two phosphorothioate internucleotide linkage modifications within position 1-5 (counting from the 3′-end) of the sense strand, and one phosphorothioate internucleotide linkage modification at positions 1 and 2 (counting from the 5′-end) and one phosphorothioate internucleotide linkage modification within positions 1-5 (counting from the 3′-end) of the antisense strand.

In some embodiments, the oligonucleotide comprises one phosphorothioate internucleotide linkage modification within position 1-5 (counting from the 5′-end) and one phosphorothioate internucleotide linkage modification within position 1-5 (counting from the 3′-end) of the sense strand, and two phosphorothioate internucleotide linkage modifications at positions 1 and 2 (counting from the 5′-end) and two phosphorothioate internucleotide linkage modifications within positions 1-5 (counting from the 3′-end) of the antisense strand.

In some embodiments, the oligonucleotide comprises one phosphorothioate internucleotide linkage modification within position 1-5 (counting from the 5′-end) and one within position 1-5 (counting from the 3′-end) of the sense strand, and two phosphorothioate internucleotide linkage modification at positions 1 and 2 (counting from the 5′-end) and one phosphorothioate internucleotide linkage modification within positions 1-5 (counting from the 3′-end) of the antisense strand.

In some embodiments, the oligonucleotide comprises one phosphorothioate internucleotide linkage modification within position 1-5 (counting from the 5′-end) of the sense strand, and two phosphorothioate internucleotide linkage modifications at positions 1 and 2 (counting from the 5′-end) and one phosphorothioate internucleotide linkage modification within positions 1-5 (counting from the 3′-end) of the antisense strand.

In some embodiments, the oligonucleotide comprises two phosphorothioate internucleotide linkage modifications within position 1-5 (counting from the 5′-end) of the sense strand, and one phosphorothioate internucleotide linkage modification at positions 1 and 2 (counting from the 5′-end) and two phosphorothioate internucleotide linkage modifications within positions 1-5 (counting from the 3′-end) of the antisense strand.

In some embodiments, the oligonucleotide comprises two phosphorothioate internucleotide linkage modifications within position 1-5 (counting from the 5′-end) and one within position 1-5 (counting from the 3′-end) of the sense strand, and two phosphorothioate internucleotide linkage modifications at positions 1 and 2 (counting from the 5′-end) and one phosphorothioate internucleotide linkage modification within positions 1-5 (counting from the 3′-end) of the antisense strand.

In some embodiments, the oligonucleotide comprises two phosphorothioate internucleotide linkage modifications within position 1-5 (counting from the 5′-end) and one phosphorothioate internucleotide linkage modification within position 1-5 (counting from the 3′-end) of the sense strand, and two phosphorothioate internucleotide linkage modifications at positions 1 and 2 (counting from the 5′-end) and two phosphorothioate internucleotide linkage modifications within positions 1-5 (counting from the 3′-end) of the antisense strand.

In some embodiments, the oligonucleotide comprises two phosphorothioate internucleotide linkage modifications within position 1-5 (counting from the 5′-end) and one phosphorothioate internucleotide linkage modification within position 1-5 (counting from the 3′-end) of the sense strand, and one phosphorothioate internucleotide linkage modification at positions 1 and 2 (counting from the 5′-end) and two phosphorothioate internucleotide linkage modifications within positions 1-5 (counting from the 3′-end) of the antisense strand.

In some embodiments, the oligonucleotide comprises two phosphorothioate internucleotide linkage modifications at position 1 and 2 (counting from the 5′-end), and two phosphorothioate internucleotide linkage modifications at position 1 and 2 (counting from the 3′-end) of the sense strand (counting from the 5′-end), and one phosphorothioate internucleotide linkage modification at positions 1 (counting from the 5′-end) and one at position 1 or 2 (counting from the 3′-end) of the antisense strand.

In some embodiments, the oligonucleotide comprises one phosphorothioate internucleotide linkage modification at position 1 (counting from the 5′-end), and one phosphorothioate internucleotide linkage modification at position 1 (counting from the 3′-end) of the sense strand, and two phosphorothioate internucleotide linkage modifications at positions 1 and 2 (counting from the 5′-end) and two phosphorothioate internucleotide linkage modifications at positions 1 and 2 (counting from the 3′-end) the antisense strand.

In some embodiments, the oligonucleotide comprises two phosphorothioate internucleotide linkage modifications at position 1 and 2 (counting from the 5′-end), and two phosphorothioate internucleotide linkage modifications at position 1 and 2 (counting from the 3′-end) of the sense strand, and one phosphorothioate internucleotide linkage modification at positions 1 (counting from the 5′-end) and one phosphorothioate internucleotide linkage modification at position 1 (counting from the 3′-end) of the antisense strand.

In some embodiments, the oligonucleotide comprises one phosphorothioate internucleotide linkage modification at position 1 (counting from the 5′-end), and one phosphorothioate internucleotide linkage modification at position 1 (counting from the 3′-end) of the sense strand, and two phosphorothioate internucleotide linkage modifications at positions 1 and 2 (counting from the 5′-end) and two phosphorothioate internucleotide linkage modifications at positions 1 and 2 (counting from the 5′-end) the antisense strand.

In some embodiments, the oligonucleotide comprises two phosphorothioate internucleotide linkage modifications at position 1 and 2 (counting from the 5′-end), and two phosphorothioate internucleotide linkage modifications at position 1 and 2 (counting from the 3′-end) of the sense strand, and one phosphorothioate internucleotide linkage modification at positions 1 (counting from the 5′-end) and one phosphorothioate internucleotide linkage modification at position 1 (counting from the 3′-end) of the antisense strand.

In some embodiments, the oligonucleotide one phosphorothioate internucleotide linkage modification at position 1 (counting from the 5′-end), and one phosphorothioate internucleotide linkage modification at position 1 (counting from the 3′-end) of the sense strand, and two phosphorothioate internucleotide linkage modifications at positions 1 and 2 (counting from the 5′-end) and two phosphorothioate internucleotide linkage modifications at positions 1 and 2 (counting from the 3′-end) of the antisense strand.

In some exemplary oligonucleotides, the sense strand can comprise 0, 1, 2, 3 or 4 phosphorothioate internucleotide linkages. For example, the sense strand comprises phosphorothioate internucleotide linkages between nucleotide positions 1 and 2, and between nucleotide positions 2 and 3 (counting from the 5′-end).

In some exemplary oligonucleotides, the antisense strand can comprise 1, 2, 3 or 4 phosphorothioate internucleotide linkages. For example, the sense strand comprises phosphorothioate internucleotide linkages between nucleotide positions 1 and 2, and between nucleotide positions 2 and 3 (counting from the 3′-end). In an additional example, the antisense strand comprises phosphorothioate internucleotide linkages between nucleotide positions 1 and 2 (counting from the 5′-end), between nucleotide positions 2 and 3 (counting from the 5′-end), between nucleotide positions 1 and 2 (counting from the 3′-end), and between nucleotide positions 2 and 3 (counting from the 3′-end).

In some embodiments, the sense strand comprises phosphorothioate internucleotide linkages between nucleotide positions 1 and 2 (counting from the 5′-end), and between nucleotide positions 2 and 3 (counting from the 5′-end), and the antisense strand comprises phosphorothioate internucleotide linkages between nucleotide positions 1 and 2 (counting from the 3′-end), and between nucleotide positions 2 and 3 (counting from the 5′-end). For example, the sense strand comprises phosphorothioate internucleotide linkages between nucleotide positions 1 and 2 (counting from the 5′-end), and between nucleotide positions 2 and 3 (counting from the 5′-end), and the antisense strand comprises phosphorothioate internucleotide linkages between nucleotide positions 1 and 2 (counting from the 5′-end), between nucleotide positions 2 and 3 (counting from the 5′-end), between nucleotide positions 1 and 2 (counting from the 3′-end), and between nucleotide positions 2 and 3 (counting from the 5′-end).

### 5′-Modifications

In some embodiments, the oligonucleotide can be 5′ phosphorylated or include a phosphoryl analog at the 5′ terminus. Exemplary 5′-phosphate modifications include those which are compatible with RISC mediated gene silencing. Suitable modifications include: 5′-monophosphate ((HO)₂(O)P—O-5′); 5′-diphosphate ((HO)₂(O)P—O—P(HO)(O)—O-5′); 5′-triphosphate ((HO)₂(O)P—O—(HO)(O)P—O—P(HO)(O)—O-5′); 5′-guanosine cap (7-methylated or non-methylated) (7m-G-O-5′-(HO)(O)P—O—(HO)(O)P—O—P(HO)(O)—O-5′); 5′-adenosine cap (Appp), and any modified or unmodified nucleotide cap structure (N—O-5′-(HO)(O)P—O—(HO)(O)P—O—P(HO)(O)—O-5′); 5′-monothiophosphate (phosphorothioate; (HO)₂(S)P—O-5′); 5′-monodithiophosphate (phosphorodithioate; (HO)(HS)(S)P—O-5′), 5′-phosphorothiolate ((HO)₂(O)P—S-5′); any additional combination of oxygen/sulfur replaced monophosphate, diphosphate and triphosphates (e.g. 5′-alpha-thiotriphosphate, 5′-gamma-thiotriphosphate, etc.), 5′-phosphoramidates ((HO)₂(O)P—NH-5′, (HO)(NH₂)(O)P—O-5′), 5′-alkylphosphonates (R=alkyl=methyl, ethyl, isopropyl, propyl, etc., e.g. RP(OH)(O)—O-5′-, 5′-alkenylphosphonates (i.e. vinyl, substituted vinyl), (OH)₂(O)P-5′-CH2—), 5′-alkyletherphosphonates (R=alkylether=methoxymethyl (MeOCH2—), ethoxymethyl, etc., e.g. RP(OH)(O)—O-5′—). The modification can in placed in the antisense strand of an oligonucleotide. For example, the antisense strand can comprise a 5′-vinylphosphonate nucleotide at 5′-end.

In some embodiments, the antisense comprises 5′-E-vinylphosphonate. In some embodiments, the antisense strand comprises 5′-E-vinylphosphonate and a nucleoside at position N−1 that reduces or inhibits activity of siRNA relative to a siRNA having the same antisense strand sequence, but unmodified N−1 position and a nucleoside at position N−1 that reduces or inhibits activity of siRNA relative to a siRNA having the same antisense strand sequence, but unmodified N−1 position

In some embodiments, the sense strand comprises a 5′-morpholino, a 5′-dimethylamino, a 5′-deoxy, an inverted abasic, or an inverted abasic locked nucleic acid modification at the 5′-end.

The linker between the Ig and the oligonucleotide can be attached to the sense strand, antisense strand or both strands. Further, the linker can be conjugated at the 3′-end, 5′-end or both ends of a strand. For instance, the linker can be conjugated to the sense strand. In some embodiments, the linker is conjugated to the 3′-end of the sense strand. In some other embodiments, the linker is conjugated to the 3′-end of the sense strand.

Generally, the double-stranded oligonucleotide has a melting temperature in the range from about 40° C. to about 80° C. For example, the double-stranded oligonucleotide has a melting temperature with a lower end of the range from about 40° C., 45° C., 50° C., 55° C., 60° C. or 65° C., and upper end of the range from about 70° C., 75° C. or 80° C. In some embodiments, the double-stranded oligonucleotide has a melting temperature in the range from about 55° C. to about 70° C. or in the range from about 60° C. to about 75° C. In some embodiments, the double-stranded oligonucleotide has a melting temperature in the range from about 57° C. to about 67° C. In some particular embodiments, the double-stranded oligonucleotide has a melting temperature in the range from about 60° C. to about 67° C. In some additional embodiments, the double-stranded oligonucleotide has a melting temperature in the range from about 62° C. to about 66° C.

Without wishing to be bound by a theory, thermally destabilizing modifications in the seed region of the antisense strand (i.e., at positions 2-9 from the 5′-end of the antisense strand) can reduce or inhibit off-target gene silencing. Accordingly, in some embodiments, the antisense strand comprises at least one (e.g., one, two, three, four, five or more) thermally destabilizing modification of the duplex within the first 9 nucleotide positions of the 5′ region of the antisense strand. The term “thermally destabilizing modification(s)” includes modification(s) that would result with a dsRNA with a lower overall melting temperature (Tm) (preferably a Tm with one, two, three or four degrees lower than the Tm of the dsRNA without having such modification(s).

In some embodiments, thermally destabilizing modification is located at position 2, 3, 4, 5, 6, 7, 8 or 9, or preferably at position 4, 5, 6, 7, or 8, from the 5′-end of the antisense strand. In some embodiments, the thermally destabilizing modification is located at position 2, 3, 4, 5 or 9 from the 5′-end of the antisense strand. In some other embodiments, the thermally destabilizing modification is located at position 6, 7 or 8 from the 5′-end of the antisense strand. In some particular embodiments, the thermally destabilizing modification is located at position 7 from the 5′-end of the antisense strand.

The thermally destabilizing modifications can include, but are not limited to, abasic modifications; mismatch with the opposing nucleotide in the opposing strand; and sugar modification such as 2′-deoxy modification or acyclic nucleotide, e.g., unlocked nucleic acids (UNA) or glycol nucleic acid (GNA).

Exemplary abasic modifications include, but are not limited to, the following:

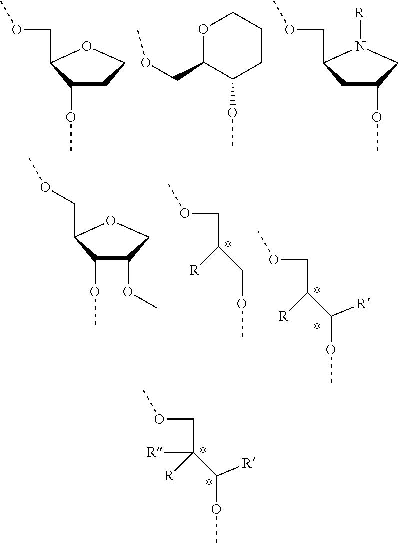

wherein R is H, Me, Et or OMe; R′ is H, Me, Et or OMe; R″ is H, Me, Et or OMe; and * represents either R, S or racemic.

Exemplary destabilizing sugar modifications include, but are not limited to the following:

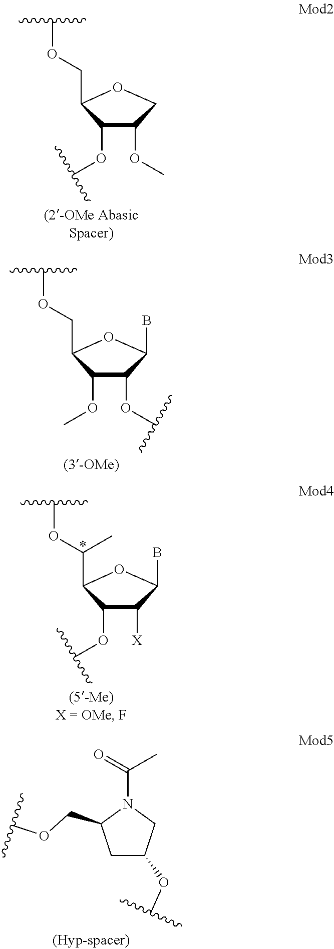

wherein B is a modified or unmodified nucleobase.

Additional sugar modifications include, but are not limited to the following:

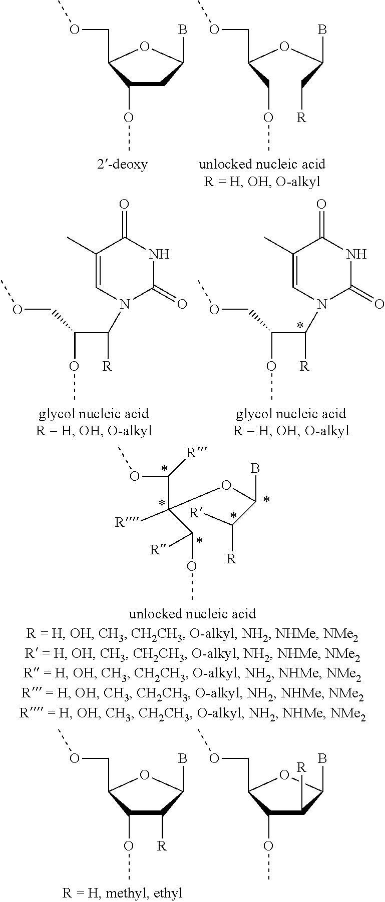

wherein B is a modified or unmodified nucleobase.

In some embodiments the thermally destabilizing modification is selected from the group consisting of:

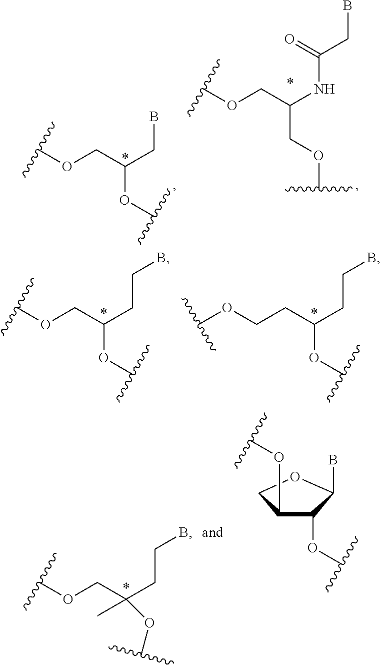

wherein B is a modified or unmodified nucleobase and the asterisk on each structure represents either R, S or racemic.

The term “acyclic nucleotide” refers to any nucleotide having an acyclic ribose sugar, for example, where any of bonds between the ribose carbons (e.g., C1′-C2′, C2′-C3′, C3′-C4′, C4′—O4′, or C1′—O4′) is absent and/or at least one of ribose carbons or oxygen (e.g., C1′, C2′, C3′, C4′ or O4′) are independently or in combination absent from the nucleotide. In some embodiments, acyclic nucleotide is

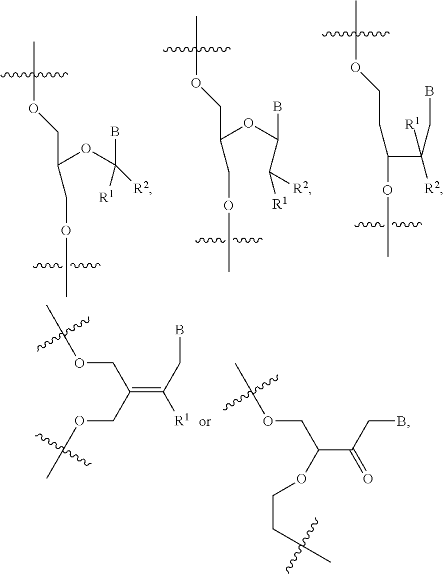

wherein B is a modified or unmodified nucleobase, R¹ and R² independently are H, halogen, OR₃, or alkyl; and R₃ is H, alkyl, cycloalkyl, aryl, aralkyl, heteroaryl or sugar). The term “UNA” refers to unlocked acyclic nucleic acid, wherein any of the bonds of the sugar has been removed, forming an unlocked “sugar” residue. In one example, UNA also encompasses monomers with bonds between C1′-C4′ being removed (i.e., the covalent carbon-oxygen-carbon bond between the C1′ and C4′ carbons). In another example, the C2′-C3′ bond (i.e., the covalent carbon-carbon bond between the C2′ and C3′ carbons) of the sugar is removed (see Mikhailov et. al., Tetrahedron Letters, 26 (17): 2059 (1985); and Fluiter et al., Mol. Biosyst., 10: 1039 (2009), which are hereby incorporated by reference in their entirety). The acyclic derivative provides greater backbone flexibility without affecting the Watson-Crick pairings. The acyclic nucleotide can be linked via 2′-5′ or 3′-5′ linkage.

The term ‘GNA’ refers to glycol nucleic acid which is a polymer similar to DNA or RNA but differing in the composition of its “backbone” in that is composed of repeating glycerol units linked by phosphodiester bonds:

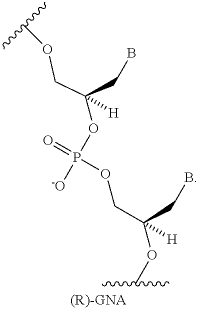

The thermally destabilizing modification of the duplex can be mismatches (i.e., noncomplementary base pairs) between the thermally destabilizing nucleotide and the opposing nucleotide in the opposite strand within the dsRNA duplex. Exemplary mismatch base pairs include G:G, G:A, G:U, G:T, A:A, A:C, C:C, C:U, C:T, U:U, T:T, U:T, or a combination thereof. Other mismatch base pairings known in the art are also amenable to the present invention. A mismatch can occur between nucleotides that are either naturally occurring nucleotides or modified nucleotides, i.e., the mismatch base pairing can occur between the nucleobases from respective nucleotides independent of the modifications on the ribose sugars of the nucleotides. In certain embodiments, the oligonucleotide comprises at least one nucleobase in the mismatch pairing that is a 2′-deoxy nucleobase; e.g., the 2′-deoxy nucleobase is in the sense strand.

In some embodiments, the thermally destabilizing modification in the seed region of the antisense strand includes nucleotides with impaired W—C H-bonding to complementary base on the target mRNA. Exemplary, nucleotides with impaired W—C H-bonding to complementary base on the target mRNA include, but are not limited to, nucleotides comprising a nucleobase independently selected from the following:

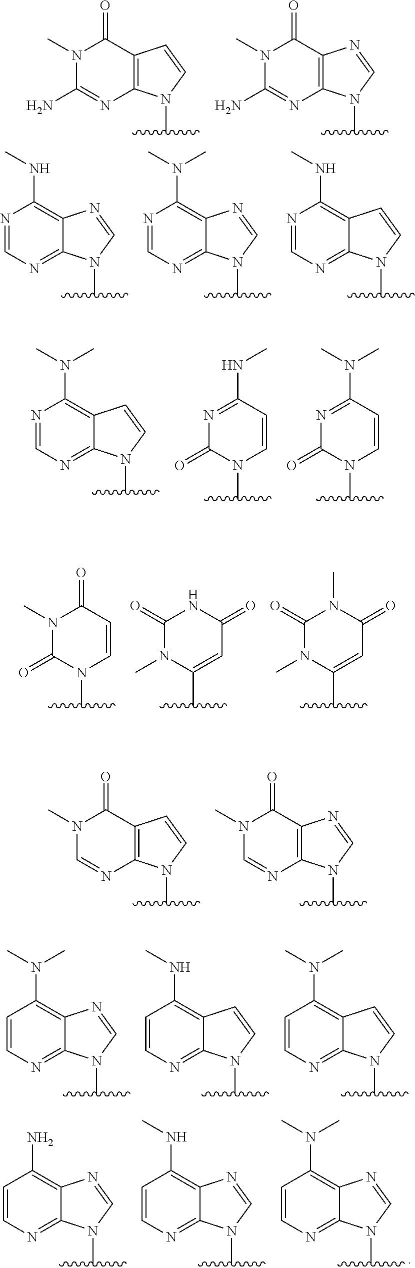

Additional examples of abasic nucleotide, acyclic nucleotide modifications (including UNA and GNA), and mismatch modifications have been described in detail in WO 2011/133876, which is herein incorporated by reference in its entirety.

The thermally destabilizing modifications can also include a universal nucleobase with reduced or abolished capability to form hydrogen bonds with the opposing bases, and phosphate modifications.

In some embodiments, the thermally destabilizing modification includes nucleotides with non-canonical bases such as, but not limited to, nucleobase modifications with impaired or completely abolished capability to form hydrogen bonds with bases in the opposite strand. These nucleobase modifications have been evaluated for destabilization of the central region of the dsRNA duplex as described in WO 2010/0011895, which is herein incorporated by reference in its entirety. Exemplary such nucleobase modifications are:

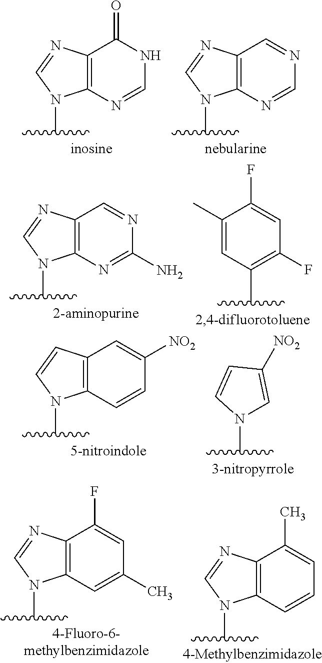

In some embodiments, the thermally destabilizing modification includes one or more □-nucleotide complementary to the base on the target mRNA, such as:

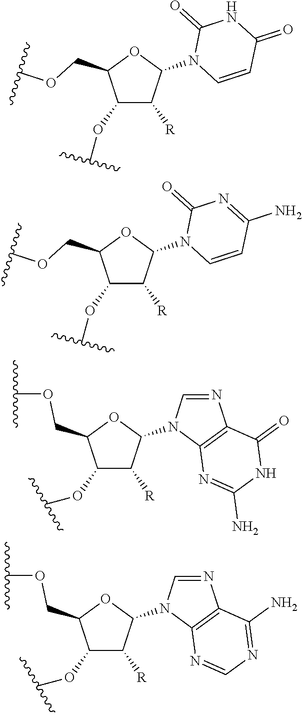

wherein R is H, OH, OCH₃, F, NH₂, NHMe, NMe₂ or O-alkyl

Exemplary phosphate modifications known to decrease the thermal stability of dsRNA duplexes compared to natural phosphodiester linkages include, but are not limited to, the following:

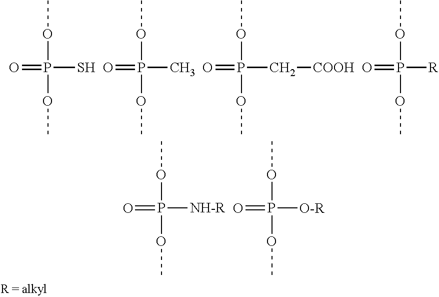

The alkyl for the R group can be a C₁-C₆alkyl. Specific alkyls for the R group include, but are not limited to methyl, ethyl, propyl, isopropyl, butyl, pentyl and hexyl.

In some embodiments, the destabilizing modification is selected from the following:

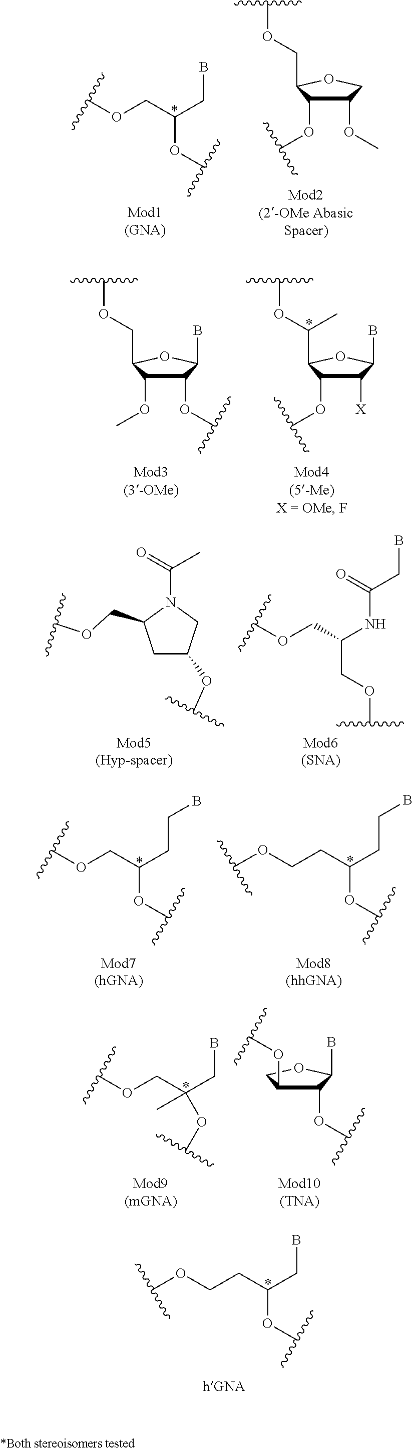

In some embodiments, the antisense strand comprises at least one stabilizing modification adjacent to the destabilizing modification. For example, the stabilizing modification can be the nucleotide at the 5′-end or the 3′-end of the destabilizing modification, i.e., at position −1 or +1 from the position of the destabilizing modification. In some embodiments, the antisense strand comprises a stabilizing modification at each of the 5′-end and the 3′-end of the destabilizing modification, i.e., positions −1 and +1 from the position of the destabilizing modification.

In some embodiments, the antisense strand comprises at least two stabilizing modifications at the 3′-end of the destabilizing modification, i.e., at positions +1 and +2 from the position of the destabilizing modification.

In some embodiments, the sense strand does not comprise a thermally stabilizing modification in position opposite or complimentary to the thermally destabilizing modification of the duplex in the antisense strand.

In some embodiments, the antisense strand comprises at least one 2′-fluoro nucleotide adjacent to the destabilizing modification. For example, the 2′-fluoro nucleotide can be the nucleotide at the 5′-end or the 3′-end of the destabilizing modification, i.e., at position −1 or +1 from the position of the destabilizing modification. In some embodiments, the antisense strand comprises a 2′-fluoro nucleotide at each of the 5′-end and the 3′-end of the destabilizing modification, i.e., positions −1 and +1 from the position of the destabilizing modification.

In some embodiments, the antisense strand comprises at least two 2′-fluoro nucleotides at the 3′-end of the destabilizing modification, i.e., at positions +1 and +2 from the position of the destabilizing modification.

In some embodiments, the sense strand does not comprise a 2′-fluoro nucleotide in position opposite or complimentary to the thermally destabilizing modification of the duplex in the antisense strand.

In some embodiments, every nucleotide in the sense strand and/or the antisense strand can be modified. Each nucleotide can be modified with the same or different modification which can include one or more alteration of one or both of the non-linking phosphate oxygens and/or of one or more of the linking phosphate oxygens; alteration of a constituent of the ribose sugar, e.g., of the 2′ hydroxyl on the ribose sugar; wholesale replacement of the phosphate moiety with “dephospho” linkers; modification or replacement of a naturally occurring base; and replacement or modification of the ribose-phosphate backbone.

As nucleic acids are polymers of monomers, many of the modifications occur at a position which is repeated within a nucleic acid, e.g., a modification of a base, or a phosphate moiety, or a non-linking O of a phosphate moiety. In some cases, the modification will occur at all of the subject positions in the nucleic acid but in many cases it will not. By way of example, a modification may only occur at a 3′ or 5′ terminal position, may only occur in a terminal region, e.g., at a position on a terminal nucleotide or in the last 2, 3, 4, 5, or 10 nucleotides of a strand. A modification may occur in a double strand region, a single strand region, or in both. A modification may occur only in the double strand region of an RNA or may only occur in a single strand region of an RNA. For example, a phosphorothioate modification at a non-linking O position may only occur at one or both termini, may only occur in a terminal region, e.g., at a position on a terminal nucleotide or in the last 2, 3, 4, 5, or 10 nucleotides of a strand, or may occur in double strand and single strand regions, particularly at termini. The 5′ end or ends can be phosphorylated.

It may be possible, e.g., to enhance stability, to include particular bases in overhangs, or to include modified nucleotides or nucleotide surrogates, in single strand overhangs, e.g., in a 5′ or 3′ overhang, or in both. E.g., it can be desirable to include purine nucleotides in overhangs. In some embodiments all or some of the bases in a 3′ or 5′ overhang may be modified, e.g., with a modification described herein. Modifications can include, e.g., the use of modifications at the 2′ position of the ribose sugar with modifications that are known in the art, e.g., the use of deoxyribonucleotides, 2′-deoxy-2′-fluoro (2′-F) or 2′-O-methyl modified instead of the ribosugar of the nucleobase, and modifications in the phosphate group, e.g., phosphorothioate modifications. Overhangs need not be homologous or orthologous with the target sequence.

In some embodiments, each residue of the sense strand and antisense strand is independently modified with LNA, HNA, CeNA, 2′-methoxyethyl, 2′-O-methyl, 2′-O-allyl, 2′-C-allyl, 2′-deoxy, or 2′-fluoro. The strands can contain more than one modification. In some embodiments, each residue of the sense strand and antisense strand is independently modified with 2′-O-methyl or 2′-fluoro. It is to be understood that these modifications are in addition to the at least one thermally destabilizing modification of the duplex present in the antisense strand.

At least two different modifications are typically present on the sense strand and antisense strand. Those two modifications may be the 2′-deoxy, 2′-O-methyl or 2′-fluoro modifications, acyclic nucleotides or others. In some embodiments, the sense strand and antisense strand each comprises two differently modified nucleotides selected from 2′-O-methyl or 2′-deoxy. In some embodiments, each residue of the sense strand and antisense strand is independently modified with a 2′-O-methyl nucleotide, 2′-deoxy nucleotide, 2′-deoxy-2′-fluoro nucleotide, 2′-O—N-methylacetamido (2′-O-NMA) nucleotide, a 2′-O-dimethylaminoethoxyethyl (2′-O-DMAEOE) nucleotide, 2′-O-aminopropyl (2′-O-AP) nucleotide, or 2′-ara-F nucleotide. Again, it is to be understood that these modifications are in addition to the at least one thermally destabilizing modification of the duplex present in the antisense strand.

In some embodiments, the oligonucleotide comprises modifications of an alternating pattern, particular in the B1, B2, B3, B1′, B2′, B3′, B4′ regions. The term “alternating motif” or “alternative pattern” as used herein refers to a motif having one or more modifications, each modification occurring on alternating nucleotides of one strand. The alternating nucleotide may refer to one per every other nucleotide or one per every three nucleotides, or a similar pattern. For example, if A, B and C each represent one type of modification to the nucleotide, the alternating motif can be “ABABABABABAB . . . ,” “AABBAABBAABB . . . ,” “AABAABAABAAB . . . ,” “AAABAAABAAAB . . . ,” “AAABBBAAABBB . . . ,” or “ABCABCABCABC . . . ,” etc.

The type of modifications contained in the alternating motif may be the same or different. For example, if A, B, C, D each represent one type of modification on the nucleotide, the alternating pattern, i.e., modifications on every other nucleotide, may be the same, but each of the sense strand or antisense strand can be selected from several possibilities of modifications within the alternating motif such as “ABABAB . . . ”, “ACACAC . . . ” “BDBDBD . . . ” or “CDCDCD . . . ,” etc.

In some embodiments, the oligonucleotide comprises the modification pattern for the alternating motif on the sense strand relative to the modification pattern for the alternating motif on the antisense strand is shifted. The shift may be such that the modified group of nucleotides of the sense strand corresponds to a differently modified group of nucleotides of the antisense strand and vice versa. For example, the sense strand when paired with the antisense strand in the dsRNA duplex, the alternating motif in the sense strand may start with “ABABAB” from 5′-3′ of the strand and the alternating motif in the antisense strand may start with “BABABA” from 3′-5′ of the strand within the duplex region. As another example, the alternating motif in the sense strand may start with “AABBAABB” from 5′-3′ of the strand and the alternating motif in the antisense strand may start with “BBAABBAA” from 3′-5′ of the strand within the duplex region, so that there is a complete or partial shift of the modification patterns between the sense strand and the antisense strand.

In some embodiments, the oligonucleotide comprises mismatch(es) with the target, within the duplex, or combinations thereof. The mismatch can occur in the overhang region or the duplex region. The base pair can be ranked on the basis of their propensity to promote dissociation or melting (e.g., on the free energy of association or dissociation of a particular pairing, the simplest approach is to examine the pairs on an individual pair basis, though next neighbor or similar analysis can also be used). In terms of promoting dissociation: A:U is preferred over G:C; G:U is preferred over G:C; and I:C is preferred over G:C (I=inosine). Mismatches, e.g., non-canonical or other than canonical pairings (as described elsewhere herein) are preferred over canonical (A:T, A:U, G:C) pairings; and pairings which include a universal base are preferred over canonical pairings.

In some embodiments, the oligonucleotide comprises at least one of the first 1, 2, 3, 4, or 5 base pairs within the duplex regions from the 5′-end of the antisense strand can be chosen independently from the group of: A:U, G:U, J:C, and mismatched pairs, e.g., non-canonical or other than canonical pairings or pairings which include a universal base, to promote the dissociation of the antisense strand at the 5′-end of the duplex.

In some embodiments, the nucleotide at the 1 position within the duplex region from the 5′-end in the antisense strand is selected from the group consisting of A, dA, dU, U, and dT. Alternatively, at least one of the first 1, 2 or 3 base pair within the duplex region from the 5′-end of the antisense strand is an AU base pair. For example, the first base pair within the duplex region from the 5′-end of the antisense strand is an AU base pair.

Without wishing to be bound by a theory, introducing 4′-modified and/or 5′-modified nucleotides to the 3′-end of a phosphodiester (PO), phosphorothioate (PS), and/or phosphorodithioate (PS2) linkage of a dinucleotide at any position of single stranded or double stranded oligonucleotide can exert steric effect to the internucleotide linkage and, hence, protecting or stabilizing it against nucleases.

In some embodiments, 5′-modified nucleoside is introduced at the 3′-end of a dinucleotide at any position of the oligonucleotide. For instance, a 5′-alkylated nucleoside can be introduced at the 3′-end of a dinucleotide at any position of the dsRNA. The alkyl group at the 5′ position of the ribose sugar can be a racemic or enantiomerically pure R or S isomer. An exemplary 5′-alkylated nucleoside is a 5′-methyl nucleoside. The 5′-methyl can be either a racemic or enantiomerically pure R or S isomer.

In some embodiments, a 4′-modified nucleoside is introduced at the 3′-end of a dinucleotide at any position of the dsRNA. For instance, a 4′-alkylated nucleoside may be introduced at the 3′-end of a dinucleotide at any position of dsRNA. The alkyl group at the 4′ position of the ribose sugar can be a racemic or enantiomerically pure R or S isomer. An exemplary 4′-alkylated nucleoside is a 4′-methyl nucleoside. The 4′-methyl can be either racemic or enantiomerically pure R or S isomer. Alternatively, a 4′-O-alkylated nucleoside may be introduced at the 3′-end of a dinucleotide at any position of single stranded or double stranded siRNA. The 4′-O-alkyl of the ribose sugar can be a racemic or enantiomerically pure R or S isomer. An exemplary 4′-O-alkylated nucleoside is a 4′-O-methyl nucleoside. The 4′-O-methyl can be either a racemic or enantiomerically pure R or S isomer.

In some embodiments, a 5′-alkylated nucleoside is introduced at any position on the sense strand or antisense strand of the dsRNA, and such modification maintains or improves potency of the dsRNA. The 5′-alkyl can be either a racemic or enantiomerically pure R or S isomer. An exemplary 5′-alkylated nucleoside is a 5′-methyl nucleoside. The 5′-methyl can be either a racemic or enantiomerically pure R or S isomer.

In some embodiments, a 4′-alkylated nucleoside is introduced at any position on the sense strand or antisense strand of the dsRNA, and such modification maintains or improves potency of the dsRNA. The 4′-alkyl can be either a racemic or enantiomerically pure R or S isomer. An exemplary 4′-alkylated nucleoside is a 4′-methyl nucleoside. The 4′-methyl can be either a racemic or enantiomerically pure R or S isomer.

In some embodiments, a 4′-O-alkylated nucleoside is introduced at any position on the sense strand or antisense strand of the dsRNA, and such modification maintains or improves potency of the dsRNA. The 5′-alkyl can be either a racemic or enantiomerically pure R or S isomer. An exemplary 4′-O-alkylated nucleoside is a 4′-O-methyl nucleoside. The 4′-O-methyl can be either a racemic or enantiomerically pure R or S isomer.

In some embodiments, the oligonucleotide can comprise 2′-5′ linkages (with 2′-H, 2′-OH and 2′-OMe and with P═O or P═S). For example, the 2′-5′ linkages modifications can be used to promote nuclease resistance or to inhibit binding of the sense to the antisense strand, or can be used at the 5′ end of the sense strand to avoid sense strand activation by RISC. In some embodiments, the sense strand comprises a 2′-5′-linkage between positions N−1 and N-2, counting from 5′-end.

In some embodiments, the oligonucleotide can comprise L sugars (e.g., L ribose, L-arabinose with 2′-H, 2′-OH and 2′-OMe). For example, these L sugars modifications can be used to promote nuclease resistance or to inhibit binding of the sense to the antisense strand, or can be used at the 5′ end of the sense strand to avoid sense strand activation by RISC. In some embodiments, the sense strand comprises a L sugar nucleotide at the 5′-end.

### Methods

In another aspect, the disclosure provides methods of using the polypeptides, polynucleotides and/or the oligonucleotides described herein. For example, provided herein is a method for modifying a target RNA. Generally, the method comprises contacting a target molecule (a) with a polypeptide describe herein and an oligonucleotide described herein; or (b) with a polypeptide describe herein complexed with an oligonucleotide described herein. At least a portion (e.g., 15-30 nucleotides long) of the oligonucleotide comprises a nucleotide sequence that is substantially complementary to a target sequence. In some embodiments, said portion of the oligonucleotide comprises a mismatch (e.g., an A:C mismatch) with the target sequence. For example, the oligonucleotide comprises a C at a position complementary to an A in the target sequence.

The target RNA can be any desired RNA molecule, including, but not limited to, messenger RNA (mRNA), transfer RNA (tRNA), ribosomal RNA (rRNA) and microRNA (miRNA). In some preferred embodiments, the target RNA is a mRNA.

In some embodiments, the target RNA sequence comprises a sequence associated with a disease or disorder. In some embodiments, the target RNA sequence comprises a point mutation associated with a disease or disorder. For example, the target RNA sequence comprises a G→A point mutation associate with a disease or disorder. In another example, the target RNA sequence comprises a C→T point mutation associate with a disease or disorder

In some embodiments, the target RNA sequence encodes a protein and wherein the point mutation is in a codon and results in a change in the amino acid encoded by the mutant codon as compared to the wild-type codon. In some embodiments, the modification of the target RNA results in a change of the amino acid encoded by the mutant codon. In some embodiments, the modification of the target RNA results in the codon encoding the wild-type amino acid. In some embodiments, the disease or disorder is cystic fibrosis, phenylketonuria, epidermolytic hyperkeratosis (EHK), Charcot-Marie-Toot disease type 4J, neuroblastoma (NB), von Willebrand disease (vWD), myotonia congenital, hereditary renal amyloidosis, dilated cardiomyopathy (DCM), hereditary lymphedema, familial Alzheimer's disease, HIV, Prion disease, chronic infantile neurologic cutaneous articular syndrome (CINCA), desmin-related myopathy (DRM), a neoplastic disease associated with a mutant PI3KCA protein, a mutant CTNNB1 protein, a mutant HRAS protein, or a mutant p53 protein.

In some embodiments, the contacting is in vitro. In some other embodiments, the contacting is in vivo in a subject. In some embodiments, the subject has or has been diagnosed with a disease or disorder.

The methods described herein can also be used to introduce a point mutation into a target RNA. In some embodiments, the modification of the target RNA results in the correction of a genetic defect, e.g., in the correction of a point mutation that leads to a loss of function in a gene product. In some embodiments, the genetic defect is associated with a disease or disorder, e.g., a lysosomal storage disorder or a metabolic disease, such as, for example, type I diabetes. In some embodiments, the methods provided herein are used to introduce a deactivating point mutation into a gene or allele that encodes a gene product that is associated with a disease or disorder. For example, methods described herein can be used to introduce a deactivating point mutation into an oncogene mRNA (e.g., in the treatment of a proliferative disease). A deactivating mutation may, in some embodiments, generate a premature stop codon in a coding sequence, which results in the expression of a truncated gene product, e.g., a truncated protein lacking the function of the full-length protein.

### Polynucleotide Encoding the Polypeptide

The disclosure also provides a polynucleotide encoding a polypeptide described herein. The skilled person will understand that, due to the degeneracy of the genetic code, a given polypeptide can be encoded by different polynucleotides. These “variants” are encompassed herein.

In some embodiments, a polynucleotide encoding a polypeptide described herein is comprised in a vector. In some embodiments, a nucleic acid sequence encoding a polypeptide described herein is operably linked to a vector. The term “vector”, as used herein, refers to a nucleic acid construct designed for delivery to a host cell or for transfer between different host cells. As used herein, a vector can be viral or non-viral. The term “vector” encompasses any genetic element that is capable of replication when associated with the proper control elements and that can transfer gene sequences to cells. A vector can include, but is not limited to, a cloning vector, an expression vector, a plasmid, phage, transposon, cosmid, chromosome, virus, virion, etc.

In some embodiments, the vector is recombinant, e.g., it comprises sequences originating from at least two different sources. In some embodiments of any of the aspects, the vector comprises sequences originating from at least two different species. In some embodiments of any of the aspects, the vector comprises sequences originating from at least two different genes, e.g., it comprises a fusion protein or a nucleic acid encoding an expression product which is operably linked to at least one non-native (e.g., heterologous) genetic control element (e.g., a promoter, suppressor, activator, enhancer, response element, or the like).

In some embodiments, the vector or polynucleotide described herein is codon-optimized, e.g., the native or wild-type sequence of the nucleic acid sequence has been altered or engineered to include alternative codons such that altered or engineered nucleic acid encodes the same polypeptide expression product as the native/wild-type sequence, but will be transcribed and/or translated at an improved efficiency in a desired expression system. In some embodiments, the expression system is an organism other than the source of the native/wild-type sequence (or a cell obtained from such organism). In some embodiments, the vector and/or nucleic acid sequence described herein is codon-optimized for expression in a mammal or mammalian cell, e.g., a mouse, a murine cell, or a human cell. In some embodiments, the vector and/or nucleic acid sequence described herein is codon-optimized for expression in a human cell. In some embodiments, the vector and/or nucleic acid sequence described herein is codon-optimized for expression in a yeast or yeast cell. In some embodiments, the vector and/or nucleic acid sequence described herein is codon-optimized for expression in a bacterial cell. In some embodiments, the vector and/or nucleic acid sequence described herein is codon-optimized for expression in an E. coli cell.

As used herein, the term “expression vector” refers to a vector that directs expression of an RNA or polypeptide from sequences linked to transcriptional regulatory sequences on the vector. The sequences expressed will often, but not necessarily, be heterologous to the cell. An expression vector may comprise additional elements, for example, the expression vector may have two replication systems, thus allowing it to be maintained in two organisms, for example in human cells for expression and in a prokaryotic host for cloning and amplification.

As used herein, the term “viral vector” refers to a nucleic acid vector construct that includes at least one element of viral origin and has the capacity to be packaged into a viral vector particle. The viral vector can contain the nucleic acid encoding an antibody or antigen-binding fragment thereof as described herein in place of non-essential viral genes. The vector and/or particle may be utilized for the purpose of transferring any nucleic acids into cells either in vitro or in vivo. Numerous forms of viral vectors are known in the art.

### Cells

The disclosure also provides a cell comprising a polypeptide described herein. The disclosure further provides a host cell comprising a polynucleotide described herein or a plasmid or vector described herein. As used herein, the term “cell” refers to a single cell as well as to a population of (i.e., more than one) cells. In some embodiments, the cell can also comprise an oligonucleotide described herein.

A host cell can be a prokaryotic or eukaryotic host cell. Exemplary host cells include, but are not limited to, bacterial cells, yeast cells, plant cell, animal (including insect) or human cells. The host cells can be employed in a method of producing a polypeptide described herein. Generally, the method comprises: culturing a host cell comprising a polynucleotide described herein or a plasmid or vector described herein under conditions such that the antibody or antigen-binding fragment thereof is expressed; and optionally recovering the polypeptide from the culture medium. The polypeptide can be concentrated and purified by a variety of biochemical and chromatographic methods, including methods utilizing differences in size, charge, hydrophobicity, solubility, specific affinity, etc. between the antibody or antigen-binding fragment thereof and other substances in the cell culture medium. In some embodiments, the polypeptide is secreted from the host cells.

The polypeptide described herein can be produced as recombinant molecules in prokaryotic or eukaryotic host cells, such as bacteria, yeast, plant, animal (including insect) or human cell lines or in transgenic animals. Recombinant methods of producing a polypeptide through the introduction of a vector including nucleic acid encoding the polypeptide into a suitable host cell is well known in the art, such as is described in Sambrook et al., Molecular Cloning: A Laboratory Manual, 2d Ed, Vols 1 to 8, Cold Spring Harbor, N Y (1989); M. W. Pennington and B. M. Dunn, Methods in Molecular Biology: Peptide Synthesis Protocols, Vol 35, Humana Press, Totawa, NJ (1994), contents of both of which are herein incorporated by reference.

### Kits

A polypeptide or polynucleotide described herein can be provided in a kit, e.g., as a component of a kit. For example, the kit includes (a) a polypeptide or polynucleotide, and optionally (b) informational material. In some embodiments, the kit further comprises an oligonucleotide, e.g., a double-stranded oligonucleotide described herein.

The informational material can be descriptive, instructional, marketing, or other material that relates to the methods described herein and/or the use of a polypeptide or polynucleotide described herein for the methods described herein. The informational material of the kits is not limited in its form. In some embodiments, the informational material can include information about production of the antibody, antigen binding fragment or the polynucleotide encoding the antibody or the antigen binding fragment, their molecular weight, concentration, date of expiration, batch, or production site information, and so forth. In some embodiments, the informational material relates to using the polypeptide or the polynucleotide to treat, prevent, or diagnosis of disorders and conditions.

In some embodiments, the informational material can include instructions to administer the polypeptide or the polynucleotide in a suitable manner to perform the methods described herein, e.g., in a suitable dose, dosage form, or mode of administration (e.g., a dose, dosage form, or mode of administration described herein). In another embodiment, the informational material can include instructions to administer the polypeptide or the polynucleotide to a suitable subject, e.g., a human, e.g., a human having, or at risk for, a disorder or condition needing treatment

The informational material of the kits is not limited in its form. In many cases, the informational material, e.g., instructions, is provided in print but can also be in other formats, such as computer readable material.

Components of the kit, e.g., the polypeptide, the polynucleotide and/or the oligonucleotide can be provided in any form, e.g., liquid, dried or lyophilized form. It is preferred that the polypeptide, the polynucleotide, or the oligonucleotide be substantially pure and/or sterile. When the polypeptide, the polynucleotide and/or the oligonucleotide is provided in a liquid solution, the liquid solution preferably is an aqueous solution, with a sterile aqueous solution being preferred. When the polypeptide, the polynucleotide and/or the oligonucleotide is provided as a dried form, reconstitution generally is by the addition of a suitable solvent. The solvent, e.g., sterile water or buffer, can optionally be provided in the kit.

The kit can include one or more containers for the components of the kit. In some embodiments, the kit contains separate containers, dividers, or compartments for the different components of the kit. For example, the polypeptide, the polynucleotide and/or the oligonucleotide can be contained in a bottle, vial, or syringe, and the informational material can be contained association with the container. In other embodiments, the separate elements of the kit are contained within a single, undivided container. For example, the polypeptide, the polynucleotide and/or the oligonucleotide is contained in a bottle, vial or syringe that has attached thereto the informational material in the form of a label. In some embodiments, the kit includes a plurality (e.g., a pack) of individual containers, each containing one or more-unit dosage forms of the polypeptide, the polynucleotide and/or the oligonucleotide. For example, the kit includes a plurality of syringes, ampules, foil packets, or blister packs, each containing a single unit dose of the polypeptide, the polynucleotide and/or the oligonucleotide. The containers of the kits can be airtight, waterproof (e.g., impermeable to changes in moisture or evaporation), and/or light-tight.

The kit optionally includes a device suitable for administration of the polypeptide, the polynucleotide and/or the oligonucleotide, e.g., a syringe, inhalant, dropper (e.g., eye dropper), swab (e.g., a cotton swab or wooden swab), or any such delivery device. In some embodiments, the device is an implantable device that dispenses metered doses of the polypeptide, the polynucleotide and/or the oligonucleotide. The disclosure also features a method of providing a kit, e.g., by combining components described herein.

In some embodiments, the kit can further comprise additional components and/or reagents for practicing the methods described herein using the polypeptide, the polynucleotide and/or the oligonucleotide described herein.

### Compositions

Polypeptides, polynucleotides and/or oligonucleotides described herein can be formulated in compositions. For example, polypeptides, polynucleotides and/or oligonucleotides described herein can be formulated into pharmaceutical compositions for therapeutic use. Accordingly, in another aspect, the invention provides a pharmaceutical composition comprising a polypeptide, polynucleotide and/or oligonucleotide described herein. Pharmaceutically acceptable compositions comprise a therapeutically-effective amount of one or more of the polypeptides, polynucleotides and/or oligonucleotides described herein, taken alone, or formulated together with one or more pharmaceutically acceptable carriers (additives), excipient and/or diluents.

The pharmaceutical compositions can be specially formulated for administration in solid or liquid form, including those adapted for the following: (1) oral administration, for example, drenches (aqueous or non-aqueous solutions or suspensions), tablets, e.g., those targeted for buccal, sublingual, and systemic absorption, boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; or (8) nasally. Delivery using subcutaneous or intravenous methods can be particularly advantageous.

The phrase “therapeutically-effective amount” as used herein means that amount of a compound, material, or composition comprising a conjugate described herein which is effective for producing some desired therapeutic effect in at least a sub-population of cells in an animal at a reasonable benefit/risk ratio applicable to any medical treatment.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically-acceptable material, composition, or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) lubricating agents, such as magnesium state, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; and (22) other non-toxic compatible substances employed in pharmaceutical formulations.

As used herein, a “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions. Pharmaceutical carriers include sterile aqueous solutions or dispersions and sterile powders for the extemporaneous preparation of sterile injectable solutions or dispersion. The use of such media and agents for pharmaceutically active substances is known in the art.

The formulations can conveniently be presented in unit dosage form and can be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect. Generally, out of one hundred percent, this amount will range from about 0.1 percent to about ninety-nine percent of active ingredient, preferably from about 5 percent to about 70 percent, most preferably from about 10 percent to about 30 percent.

Pharmaceutical compositions for use with the methods described herein can be formulated in a conventional manner using one or more physiologically acceptable carriers or excipients. For example, a polypeptide, polynucleotide and/or oligonucleotide described herein can be formulated for administration by, for example, by aerosol, intravenous, oral, or topical route. The compositions can be formulated for intralesional, intratumoral, intraperitoneal, subcutaneous, intramuscular, or intravenous injection; infusion; liposome-mediated delivery; topical, intrathecal, gingival pocket, per rectum, intrabronchial, nasal, transmucosal, intestinal, oral, ocular, or otic delivery.

Techniques and formulations generally can be found in Remington's Pharmaceutical Sciences, Meade Publishing Co., Easton, PA. For systemic administration, injection is preferred, including intramuscular, intravenous, intraperitoneal, and subcutaneous. For injection, polypeptide, polynucleotide and/or oligonucleotide described herein can be formulated in liquid solutions, preferably in physiologically compatible buffers such as Hank's solution or Ringer's solution. In addition, the polypeptide, polynucleotide and/or oligonucleotide can be formulated in solid form and redissolved or suspended immediately prior to use. Lyophilized forms are also included.

For oral administration, the pharmaceutical composition can take the form of, for example, tablets or capsules prepared by conventional means with pharmaceutically acceptable excipients such as binding agents (e.g., pregelatinized maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets can be coated by methods well known in the art. Liquid preparations for oral administration can take the form of, for example, solutions, syrups, or suspensions, or they may be presented as a dry product for constitution with water or other suitable vehicle before use. Such liquid preparations can be prepared by conventional means with pharmaceutically acceptable additives such as suspending agents (e.g., sorbitol syrup, cellulose derivatives, or hydrogenated edible fats); emulsifying agents (e.g., lecithin or acacia); non-aqueous vehicles (e.g., pharmaceutically acceptable oils, oily esters, ethyl alcohol or fractionated vegetable oils); and preservatives (e.g., methyl or propyl-p-hydroxybenzoates or sorbic acid). The preparations can also contain buffer salts, flavoring, coloring, and sweetening agents as appropriate.

Preparations for oral administration can be suitably formulated to give controlled release of the active compound. For buccal administration the compositions can take the form of tablets or lozenges formulated in conventional manner. For administration by inhalation, the compounds for use as described herein are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of e.g., gelatin for use in an inhaler or insufflator can be formulated containing a powder mix of the compound and a suitable powder base such as lactose or starch.

The polypeptide, polynucleotide and/or oligonucleotide can be formulated for parenteral administration by injection, e.g., by bolus injection or continuous infusion. Formulations for injection can be presented in unit dosage form, e.g., in ampoules or in multi-dose containers, with an added preservative. The compositions can take such forms as suspensions, solutions, or emulsions in oily or aqueous vehicles, and can contain formulatory agents such as suspending, stabilizing and/or dispersing agents. Alternatively, the active ingredient can be in powder form for constitution with a suitable vehicle, e.g., sterile pyrogen-free water, before use.

In addition to the formulations described previously, the polypeptide, polynucleotide and/or oligonucleotide can also be formulated as a depot preparation. Such long-acting formulations can be administered by implantation (for example subcutaneously or intramuscularly) or by intramuscular injection. Thus, for example, the antibodies can be formulated with suitable polymeric or hydrophobic materials (for example as an emulsion in an acceptable oil) or ion exchange resins, or as sparingly soluble derivatives, for example, as a sparingly soluble salt.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration bile salts and fusidic acid derivatives. In addition, detergents can be used to facilitate permeation. Transmucosal administration can be through nasal sprays or using suppositories. For topical administration, the polypeptide, polynucleotide and/or oligonucleotide can be formulated into ointments, salves, gels, or creams as generally known in the art. A wash solution can be used locally to treat an injury or inflammation to accelerate healing.

The compositions can, if desired, be presented in a pack or dispenser device which can contain one or more-unit dosage forms containing the active ingredient. The pack can for example comprise metal or plastic foil, such as a blister pack. The pack or dispenser device can be accompanied by instructions for administration.

### Liposomes and Lipid Formulations

The polypeptides, polynucleotides and/or oligonucleotides described herein can be formulated for delivery in a membranous molecular assembly, e.g., a liposome or a micelle. As used herein, the term “liposome” refers to a vesicle composed of amphiphilic lipids arranged in at least one bilayer, e.g., one bilayer or a plurality of bilayers. Liposomes include unilamellar and multilamellar vesicles that have a membrane formed from a lipophilic material and an aqueous interior. The aqueous portion contains the polypeptide, polynucleotide and/or oligonucleotide. The lipophilic material isolates the aqueous interior from an aqueous exterior, which typically does not include the polypeptide, polynucleotide and/or oligonucleotide, although in some examples, it may. Liposomes are useful for the transfer and delivery of active ingredients to the site of action. Because the liposomal membrane is structurally similar to biological membranes, when liposomes are applied to a tissue, the liposomal bilayer fuses with bilayer of the cellular membranes. As the merging of the liposome and cell progresses, the internal aqueous contents that include a polypeptide, polynucleotide or oligonucleotide described herein are delivered into the cell. In some cases, the liposomes are also specifically targeted, e.g., to direct the conjugate to particular cell types.

A liposome containing a polypeptide, polynucleotide or oligonucleotide described herein can be prepared by a variety of methods. In one example, the lipid component of a liposome is dissolved in a detergent so that micelles are formed with the lipid component. For example, the lipid component can be an amphipathic cationic lipid or lipid conjugate. The detergent can have a high critical micelle concentration and may be nonionic. Exemplary detergents include cholate, CHAPS, octylglucoside, deoxycholate, and lauroyl sarcosine. The polypeptide, polynucleotide or oligonucleotide is then added to the micelles that include the lipid component. After condensation, the detergent is removed, e.g., by dialysis, to yield a liposomal preparation.

If necessary, a carrier compound that assists in condensation can be added during the condensation reaction, e.g., by controlled addition. For example, the carrier compound can be a polymer other than a nucleic acid (e.g., spermine or spermidine). pH can also be adjusted to favor condensation.

Further description of methods for producing stable polynucleotide or oligonucleotide delivery vehicles, which incorporate a polynucleotide/cationic lipid complex as structural components of the delivery vehicle, are described in, e.g., WO 96/37194. Liposome formation can also include one or more aspects of exemplary methods described in Felgner, P. L. et al., Proc. Natl. Acad. Sci., USA 8:7413-7417, 1987; U.S. Pat. Nos. 4,897,355; 5,171,678; Bangham, et al. M. Mol. Biol. 23:238, 1965; Olson, et al. Biochim. Biophys. Acta 557:9, 1979; Szoka, et al. Proc. Natl. Acad. Sci. 75: 4194, 1978; Mayhew, et al. Biochim. Biophys. Acta 775:169, 1984; Kim, et al. Biochim. Biophys. Acta 728:339, 1983; and Fukunaga, et al. Endocrinol. 115:757, 1984, which are incorporated by reference in their entirety. Commonly used techniques for preparing lipid aggregates of appropriate size for use as delivery vehicles include sonication and freeze-thaw plus extrusion (see, e.g., Mayer, et al. Biochim. Biophys. Acta 858:161, 1986, which is incorporated by reference in its entirety). Microfluidization can be used when consistently small (50 to 200 nm) and relatively uniform aggregates are desired (Mayhew, et al. Biochim. Biophys. Acta 775:169, 1984, which is incorporated by reference in its entirety).

Liposomes that are pH-sensitive or negatively-charged entrap nucleic acid molecules rather than complex with them. Since both the nucleic acid molecules and the lipid are similarly charged, repulsion rather than complex formation occurs. Nevertheless, some nucleic acid molecules are entrapped within the aqueous interior of these liposomes. pH-sensitive liposomes have been used to deliver DNA encoding the thymidine kinase gene to cell monolayers in culture. Expression of the exogenous gene was detected in the target cells (Zhou et al., Journal of Controlled Release, 19, (1992) 269-274, which is incorporated by reference in its entirety).

One major type of liposomal composition includes phospholipids other than naturally-derived phosphatidylcholine. Neutral liposome compositions, for example, can be formed from dimyristoyl phosphatidylcholine (DMPC) or dipalmitoyl phosphatidylcholine (DPPC). Anionic liposome compositions generally are formed from dimyristoyl phosphatidylglycerol, while anionic fusogenic liposomes are formed primarily from dioleoyl phosphatidylethanolamine (DOPE). Another type of liposomal composition is formed from phosphatidylcholine (PC) such as, for example, soybean PC, and egg PC. Another type is formed from mixtures of phospholipid and/or phosphatidylcholine and/or cholesterol.

Examples of other methods to introduce liposomes into cells in vitro and include U.S. Pat. Nos. 5,283,185; 5,171,678; WO 94/00569; WO 93/24640; WO 91/16024; Felgner, J. Biol. Chem. 269:2550, 1994; Nabel, Proc. Natl. Acad. Sci. 90:11307, 1993; Nabel, Human Gene Ther. 3:649, 1992; Gershon, Biochem. 32:7143, 1993; and Strauss EMBO J. 11:417, 1992.

In some embodiments, cationic liposomes are used. Cationic liposomes possess the advantage of being able to fuse to the cell membrane.

Further advantages of liposomes include: liposomes obtained from natural phospholipids are biocompatible and biodegradable; liposomes can incorporate a wide range of water and lipid soluble drugs; liposomes can protect encapsulated polypeptides, polynucleotides, or oligonucleotides in their internal compartments from metabolism and degradation (Rosoff, in “Pharmaceutical Dosage Forms,” Lieberman, Rieger and Banker (Eds.), 1988, volume 1, p. 245). Important considerations in the preparation of liposome formulations are the lipid surface charge, vesicle size and the aqueous volume of the liposomes.

A positively charged synthetic cationic lipid, N-[1-(2,3-dioleyloxy)propyl]-N,N,N-trimethylammonium chloride (DOTMA) can be used to form small liposomes that interact spontaneously with nucleic acid to form lipid-nucleic acid complexes which are capable of fusing with the negatively charged lipids of the cell membranes of tissue culture cells.

A DOTMA analogue, 1,2-bis(oleoyloxy)-3-(trimethylammonium)propane (DOTAP) can be used in combination with a phospholipid to form DNA-complexing vesicles. Lipofectin™ Bethesda Research Laboratories, Gaithersburg, Md.) is an effective agent for the delivery of highly anionic nucleic acids into living tissue culture cells that comprise positively charged DOTMA liposomes which interact spontaneously with negatively charged polynucleotides to form complexes. When enough positively charged liposomes are used, the net charge on the resulting complexes is also positive. Positively charged complexes prepared in this way spontaneously attach to negatively charged cell surfaces, fuse with the plasma membrane, and efficiently deliver functional nucleic acids into, for example, tissue culture cells. Another commercially available cationic lipid, 1,2-bis(oleoyloxy)-3,3-(trimethylammonium)propane (“DOTAP”) (Boehringer Mannheim, Indianapolis, Indiana) differs from DOTMA in that the oleoyl moieties are linked by ester, rather than ether linkages.

Other reported cationic lipid compounds include those that have been conjugated to a variety of moieties including, for example, carboxyspermine which has been conjugated to one of two types of lipids and includes compounds such as 5-carboxyspermylglycine dioctaoleoylamide (“DOGS”) (Transfectam™, Promega, Madison, Wisconsin) and dipalmitoylphosphatidylethanolamine 5-carboxyspermyl-amide (“DPPES”) (see, e.g., U.S. Pat. No. 5,171,678).

Another cationic lipid conjugate includes derivatization of the lipid with cholesterol (“DC-Chol”) which has been formulated into liposomes in combination with DOPE (See, Gao, X. and Huang, L., Biochim. Biophys. Res. Commun. 179:280, 1991). Lipopolylysine, made by conjugating polylysine to DOPE, has been reported to be effective for transfection in the presence of serum (Zhou, X. et al., Biochim. Biophys. Acta 1065:8, 1991, which is incorporated by reference in its entirety). For certain cell lines, these liposomes containing conjugated cationic lipids, are said to exhibit lower toxicity and provide more efficient transfection than the DOTMA-containing compositions. Other commercially available cationic lipid products include DMRIE and DMRIE-HP (Vical, La Jolla, California) and Lipofectamine (DOSPA) (Life Technology, Inc., Gaithersburg, Maryland). Other cationic lipids suitable for the delivery of oligonucleotides are described in WO 98/39359 and WO 96/37194.

Liposomal formulations are particularly suited for topical administration, liposomes present several advantages over other formulations. Such advantages include reduced side effects related to high systemic absorption of the administered drug, increased accumulation of the administered drug at the desired target, and the ability to administer the polypeptide, polynucleotide and/or oligonucleotide, into the skin. In some implementations, liposomes are used for delivering polypeptide, polynucleotide and/or oligonucleotide to epidermal cells and also to enhance the penetration of polypeptide, polynucleotide and/or oligonucleotide into dermal tissues, e.g., into skin. For example, the liposomes can be applied topically. Topical delivery of drugs formulated as liposomes to the skin has been documented (see, e.g., Weiner et al., Journal of Drug Targeting, 1992, vol. 2,405-410 and du Plessis et al., Antiviral Research, 18, 1992, 259-265; Mannino, R. J. and Fould-Fogerite, S., Biotechniques 6:682-690, 1988; Itani, T. et al. Gene 56:267-276. 1987; Nicolau, C. et al. Meth. Enz. 149:157-176, 1987; Straubinger, R. M. and Papahadjopoulos, D. Meth. Enz. 101:512-527, 1983; Wang, C. Y. and Huang, L., Proc. Natl. Acad. Sci. USA 84:7851-7855, 1987, which are incorporated by reference in their entirety).

Non-ionic liposomal systems have also been examined to determine their utility in the delivery of drugs to the skin, in particular systems comprising non-ionic surfactant and cholesterol. Non-ionic liposomal formulations comprising Novasome I (glyceryl dilaurate/cholesterol/polyoxyethylene-10-stearyl ether) and Novasome II (glyceryl distearate/cholesterol/polyoxyethylene-10-stearyl ether) were used to deliver a drug into the dermis of mouse skin.

Liposomes that include a conjugate described herein can be made highly deformable. Such deformability can enable the liposomes to penetrate through pore that are smaller than the average radius of the liposome. For example, transfersomes are a type of deformable liposomes. Transfersomes can be made by adding surface edge activators, usually surfactants, to a standard liposomal composition. Transfersomes that include polypeptide, polynucleotide and/or oligonucleotide can be delivered, for example, subcutaneously by infection. In order to cross intact mammalian skin, lipid vesicles must pass through a series of fine pores, each with a diameter less than 50 nm, under the influence of a suitable transdermal gradient. In addition, due to the lipid properties, these transfersomes can be self-optimizing (adaptive to the shape of pores, e.g., in the skin), self-repairing, and can frequently reach their targets without fragmenting, and often self-loading.

Other formulations amenable to the present invention are described in U.S. provisional application Ser. No. 61/018,616, filed Jan. 2, 2008; 61/018,611, filed Jan. 2, 2008; 61/039,748, filed Mar. 26, 2008; 61/047,087, filed Apr. 22, 2008, and 61/051,528, filed May 8, 2008. PCT application no PCT/US2007/080331, filed Oct. 3, 2007, also describes formulations that are amenable to the present invention.

Surfactants. Surfactants find wide application in formulations such as emulsions (including microemulsions) and liposomes (see above). A conjugate formulation can include a surfactant. In some embodiments, a conjugate described herein is formulated as an emulsion that includes a surfactant. The most common way of classifying and ranking the properties of the many different types of surfactants, both natural and synthetic, is by the use of the hydrophile/lipophile balance (HLB). The nature of the hydrophilic group provides the most useful means for categorizing the different surfactants used in formulations (Rieger, in “Pharmaceutical Dosage Forms,” Marcel Dekker, Inc., New York, NY, 1988, p. 285).

If the surfactant molecule is not ionized, it is classified as a nonionic surfactant. Nonionic surfactants find wide application in pharmaceutical products and are usable over a wide range of pH values. In general, their HLB values range from 2 to about 18 depending on their structure. Nonionic surfactants include nonionic esters such as ethylene glycol esters, propylene glycol esters, glyceryl esters, polyglyceryl esters, sorbitan esters, sucrose esters, and ethoxylated esters. Nonionic alkanolamides and ethers such as fatty alcohol ethoxylates, propoxylated alcohols, and ethoxylated/propoxylated block polymers are also included in this class. The polyoxyethylene surfactants are the most popular members of the nonionic surfactant class.

If the surfactant molecule carries a negative charge when it is dissolved or dispersed in water, the surfactant is classified as anionic. Anionic surfactants include carboxylates such as soaps, acyl lactylates, acyl amides of amino acids, esters of sulfuric acid such as alkyl sulfates and ethoxylated alkyl sulfates, sulfonates such as alkyl benzene sulfonates, acyl isethionates, acyl taurates and sulfosuccinates, and phosphates. The most important members of the anionic surfactant class are the alkyl sulfates and the soaps.

If the surfactant molecule carries a positive charge when it is dissolved or dispersed in water, the surfactant is classified as cationic. Cationic surfactants include quaternary ammonium salts and ethoxylated amines. The quaternary ammonium salts are the most used members of this class.

If the surfactant molecule has the ability to carry either a positive or negative charge, the surfactant is classified as amphoteric. Amphoteric surfactants include acrylic acid derivatives, substituted alkylamides, N-alkylbetaines and phosphatides.

The use of surfactants in drug products, formulations and in emulsions has been reviewed (Rieger, in “Pharmaceutical Dosage Forms,” Marcel Dekker, Inc., New York, NY, 1988, p. 285).

Micelles and other Membranous Formulations. Formulations comprising a conjugate described herein can be provided as a micellar formulation. “Micelles” are defined herein as a particular type of molecular assembly in which amphipathic molecules are arranged in a spherical structure such that all the hydrophobic portions of the molecules are directed inward, leaving the hydrophilic portions in contact with the surrounding aqueous phase. The converse arrangement exists if the environment is hydrophobic.

A mixed micellar formulation suitable for delivery through transdermal membranes may be prepared by mixing an aqueous solution of the polypeptide, polynucleotide and/or oligonucleotide, an alkali metal C₈ to C₂₂ alkyl sulphate, and a micelle forming compounds. Exemplary micelle forming compounds include lecithin, hyaluronic acid, pharmaceutically acceptable salts of hyaluronic acid, glycolic acid, lactic acid, chamomile extract, cucumber extract, oleic acid, linoleic acid, linolenic acid, monoolein, monooleates, monolaurates, borage oil, evening of primrose oil, menthol, trihydroxy oxo cholanyl glycine and pharmaceutically acceptable salts thereof, glycerin, polyglycerin, lysine, polylysine, triolein, polyoxyethylene ethers and analogues thereof, polidocanol alkyl ethers and analogues thereof, chenodeoxycholate, deoxycholate, and mixtures thereof. The micelle forming compounds may be added at the same time or after addition of the alkali metal alkyl sulphate. Mixed micelles will form with substantially any kind of mixing of the ingredients but vigorous mixing in order to provide smaller size micelles.

In one method a first micellar composition is prepared which contains conjugate described herein and at least the alkali metal alkyl sulphate. The first micellar composition is then mixed with at least three micelle forming compounds to form a mixed micellar composition. In another method, the micellar composition is prepared by mixing conjugate described herein, the alkali metal alkyl sulphate and at least one of the micelle forming compounds, followed by addition of the remaining micelle forming compounds, with vigorous mixing.

Phenol and/or m-cresol may be added to the mixed micellar composition to stabilize the formulation and protect against bacterial growth. Alternatively, phenol and/or m-cresol may be added with the micelle forming ingredients. An isotonic agent such as glycerin may also be added after formation of the mixed micellar composition.

For delivery of the micellar formulation as a spray, the formulation can be put into an aerosol dispenser and the dispenser is charged with a propellant. The propellant, which is under pressure, is in liquid form in the dispenser. The ratios of the ingredients are adjusted so that the aqueous and propellant phases become one, i.e., there is one phase. If there are two phases, it is necessary to shake the dispenser prior to dispensing a portion of the contents, e.g., through a metered valve. The dispensed dose of pharmaceutical agent is propelled from the metered valve in a fine spray.

Propellants may include hydrogen-containing chlorofluorocarbons, hydrogen-containing fluorocarbons, dimethyl ether, and diethyl ether. In certain embodiments, HFA 134a (1,1,1,2 tetrafluoroethane) may be used.

The specific concentrations of the essential ingredients can be determined by relatively straightforward experimentation. For absorption through the oral cavities, it is often desirable to increase, e.g., at least double or triple, the dosage for through injection or administration through the gastrointestinal tract.

Particles. In some embodiments, conjugate described herein can be incorporated into a particle, e.g., a microparticle. Microparticles can be produced by spray-drying, but may also be produced by other methods including lyophilization, evaporation, fluid bed drying, vacuum drying, or a combination of these techniques.

Exemplary embodiments of the various aspects can be described by one or more of the following numbered embodiments:

Embodiment: 1 A system for modifying a target RNA, the system comprising: (a) a polypeptide or a nucleic acid encoding a polypeptide, wherein the polypeptide comprises: (i) a first domain comprising a catalytic domain of an RNA modifying enzyme, wherein the RNA modifying enzyme is not a nuclease; and (ii) a second domain comprising a MID domain of an Argonaute (Ago) protein; and (b) an oligonucleotide, optionally the oligonucleotide is double-stranded and comprises a double-stranded region of at least 17 base-pairs.

Embodiment 2: The system of Embodiment 1, wherein the second domain further comprises a PAZ domain of an Ago.

Embodiment 3: The system of any one of Embodiments 1-2, wherein the second domain further comprises a PIWI domain of an Ago.

Embodiment 4: The system of Embodiment 3, wherein the PIWI domain lacks nuclease activity.

Embodiment 5: The system of any one of Embodiments 1-4, wherein the Ago is a mammalian Ago.

Embodiment 6: The system of any one of Embodiments 1-5, wherein the Ago is a human Ago.

Embodiment 7: The system of any one of Embodiments 1-6, wherein Ago is Ago1, Ago2, Ago3, Ago4.

Embodiment 8 The system of any one of Embodiments 1-7, wherein the second domain comprises an amino acid sequence having a mutation at one or more positions selected from the group consisting of D597 and D699 of human Ago2 amino acid sequence, or a corresponding position in a homologous or orthologous Ago protein.

Embodiment 9: The system of any one of Embodiments 1-8, wherein the second domain comprises an amino acid sequence having a mutation at one or more positions selected from the group consisting of D597A and D699A of human Ago2 amino acid sequence, or a corresponding position in a homologous or orthologous Ago protein.

Embodiment 10: The system of any one of Embodiments 1-9, wherein the RNA modifying enzyme is an RNA deaminase, an RNA methylase, or an RNA demethylase.

Embodiment 11: The system of any one of Embodiments 1-10, wherein the catalytic domain of the RNA modifying enzyme is a deaminase domain of an RNA deaminase.

Embodiment 12: The system of Embodiment 11, wherein the RNA deaminase is Adenosine Deaminase Acting on RNA (ADAR) or a cytidine deaminase.

Embodiment 13: The system of Embodiment 13, wherein the ADAR is a mammalian ADAR.

Embodiment 14: The system of Embodiment 12 or 13, wherein the ADAR is human ADAR.

Embodiment 15: The system of any one of Embodiments 12-14, wherein the ADAR is ADAR1, ADAR2 or ADAR3.

Embodiment 16: The system of any one of Embodiments 1-15, wherein the first domain comprises an amino acid sequence having a mutation at one or more positions selected from the group consisting of T375, E448 and E488 of human ADAR2 (hADAR2) amino acid sequence, or a corresponding position in a homologous or orthologous ADAR protein.

Embodiment 17: The system of any one of Embodiments 1-16, wherein the first domain comprises an amino acid sequence having a mutation at one or more positions selected from the group consisting of T375Q, E448Q and E488Q of hADAR2 amino acid sequence, or a corresponding position in a homologous or orthologous ADAR protein.

Embodiment 18: The system of Embodiment 12, wherein the cytidine deaminase is an apolipoprotein B mRNA editing enzyme catalytic polypeptide-like (APOBEC).

Embodiment 19: The system of Embodiment 18, wherein the cytidine deaminase is selected from the group consisting of APOBEC1, APOBEC2, APOBEC3, APOBEC3A, APOBEC3B, APOBEC3C, APOBEC3D, APOBEC3E, APOBEC3F, APOBEC3G, APOBEC3H and APOBEC4.

Embodiment 20: The system of any one of claims 1-15, wherein the first domain comprises an amino acid sequence having a mutation at one or more positions selected from the group consisting of T339, R348, A353, V351, V355, T375, K376, E396, S397, E438, F442, H443, L444, Y445, T448, C451, R455, S486, Q488, R510, I520, V525, P539, G593, K594 and E1008 of hADAR2 amino acid sequence, or a corresponding position in a homologous or orthologous ADAR protein.

Embodiment 21: The system of Embodiment 20, wherein the first domain comprises an amino acid sequence having a mutation at one or more positions selected from the group consisting of V351, S370, T375, P462, S486, E488, N597 and E1008 of hADAR2 amino acid sequence, or a corresponding position in a homologous or orthologous ADAR protein.

Embodiment 22: The system of Embodiment 21, wherein the first domain comprises an amino acid sequence having a mutation at one or more positions selected from the group consisting of V351G, S370C, T375S, P462A, S486A, E488Q, N597I and E1008Q of hADAR2 amino acid sequence, or a corresponding position in a homologous or orthologous ADAR protein.

Embodiment 23: The system of any one of Embodiments 1-15, wherein the first domain comprises an amino acid sequence having a mutation at position Y132 of human APOBEC3A amino acid sequence, or a corresponding position in a homologous or orthologous APOBEC protein, optionally, the mutation is Y132R or Y132D.

Embodiment 24: The system of any one of Embodiments 1-23, wherein the polypeptide comprises a linker between the first domain and the second domain.

Embodiment 25: The system of any one of Embodiments 1-24, wherein the polypeptide further comprises a nuclear export signal (NES) sequence.

Embodiment 26: The system of Embodiment 25, wherein the NES sequence is located between the first and the second domain.

Embodiment 27: The system of any one of Embodiments 1-26, wherein the polypeptide further comprises a FLAG octapeptide.

Embodiment 28: The system of any one of Embodiments 1-27, wherein the polypeptide lacks nuclease activity.

Embodiment 29: The system of any one of Embodiments 1-28, wherein the oligonucleotide is double-stranded and comprises at least one 3′-single stranded overhang.

Embodiment 30: The system of any one of Embodiments 1-29, wherein the oligonucleotide is double-stranded and comprises a blunt end.

Embodiment 31: The system of any one of Embodiments 1-30, wherein the oligonucleotide is double-stranded and comprises a first nucleic acid strand and a second nucleic acid strand, wherein the first and second strands independently are at least 19 nucleotides in length.

Embodiment 32: The system of any one of Embodiments 1-31, wherein the oligonucleotide is double-stranded and comprises a double-stranded region of at least 19 base-pairs.

Embodiment 33: The system of any one of Embodiments 1-32, wherein the oligonucleotide is double-stranded and comprises a double-stranded region of at least 25 base-pairs.

Embodiment 34: The system of any one of Embodiments 1-33, wherein the oligonucleotide is double-stranded and comprises a strand having a nucleotide sequence substantially complementary to a target RNA and wherein said strand comprises a mismatch with the target RNA at position 10, counting from 5′-end of said strand.

Embodiment 35: The system of any one of Embodiments 1-34, wherein the oligonucleotide is double-stranded and comprises a strand having a nucleotide sequence substantially complementary to a target RNA and wherein said strand comprises a C at position 21, 22, 23, 24, 25, 26, 27 or 28, counting from 5′-end of said strand, and the strand comprises an A:C mismatch with the target RNA at position 21, 22, 23, 24, 25, 26, 27 or 28, counting from 5′-end of said strand.

Embodiment 36: The system of any one of Embodiments 1-35, wherein the oligonucleotide is double-stranded and comprises a strand having a nucleotide sequence substantially complementary to a target RNA and wherein said strand comprises a C at position 25, counting from 5′-end of said strand, and the strand comprises an A:C mismatch with the target RNA at position 25, counting from 5′-end of said strand

Embodiment 37: The system of any one of Embodiments 1-36, wherein the oligonucleotide is double-stranded and comprises a strand having a nucleotide sequence substantially complementary to a target RNA and wherein said strand comprises a C at position 4, 5, 6, 7, 8, 9 or 10, counting from 3′-end of said strand, and the strand comprises an A:C mismatch with the target RNA at position 4, 5, 6, 7, 8, 9 or 10, counting from 3′-end of said strand.

Embodiment 38: The system of any one of Embodiments 1-37, wherein the oligonucleotide is double-stranded and comprises a strand having a nucleotide sequence substantially complementary to a target RNA and wherein said strand comprises a C at position 7, counting from 3′-end of said strand, and the strand comprises an A:C mismatch with the target RNA at position 7, counting from 3′-end of said strand.

Embodiment 39: The system of any one of Embodiments 1-38, wherein the oligonucleotide comprises at least one nucleic acid modification.

Embodiment 39: The system of any one of Embodiments 1-38, wherein the oligonucleotide comprises at least one nucleic acid modification capable of inhibiting RNA interference cleavage.

Embodiment 40: A kit comprising a polypeptide or a nucleic acid encoding a polypeptide of any one of Embodiments 1-28.

Embodiment 41: The kit of Embodiment 40, wherein the kit further comprises a nucleic acid of any one of Embodiments 29-39.

Embodiment 42: A composition comprising a polypeptide or a nucleic acid encoding a polypeptide of any one of Embodiments 1-28.

Embodiment 43: The composition of Embodiment 42, wherein the composition further comprises a nucleic acid of any one of Embodiments 29-39.

Embodiment 44: A cell comprising a polypeptide or a nucleic acid encoding a polypeptide of any one of Embodiments 1-28.

Embodiment 45: The cell of Embodiment 44, wherein the cell further comprises a nucleic acid of any one of Embodiments 28-39.

Embodiment 46: A method of modifying a target RNA, the method comprising contacting the target RNA with the system of any one of Embodiments 1-39.

Embodiment 47: The method of Embodiment 46, wherein the RNA is an mRNA.

Embodiment 48: The method of Embodiment 46 or 47, wherein the RNA is in a cell.

Embodiment 49: The method of any one of Embodiments 46-48, wherein said contacting is in vitro.

Embodiment 50: The method of any one of Embodiment 46-49, wherein said contacting is in vivo.

Embodiment 51: The method of any one of Embodiments 46-50, wherein said modifying the target RNA comprises deamination of an adenosine in the target RNA.

Embodiment 52: The method of any one of Embodiments 46-50, wherein said modifying the target RNA comprises deamination of a cytidine in the target RNA.

Embodiment 53: The method of any one of Embodiments 46-50, wherein said modifying the target RNA comprises methylation of an adenosine in the target RNA.

Embodiment 54: The method of any one of Embodiments 46-50, wherein said modifying the target RNA comprises demethylation of an adenosine in the target RNA.

Embodiment 55: A cell comprising an RNA modified by any one of Embodiments 46-54.

Embodiment 56: The system of any one of Embodiments 1-33, wherein the oligonucleotide is double-stranded and comprises a strand having a nucleotide sequence substantially complementary to a target RNA, wherein the target RNA forms a loop structure when hybridized to said strand, and wherein said loop structure comprises a single-stranded C nucleotide.

Embodiment 57: The system of Embodiment 56, wherein said loop structure is 5 to 20 nucleotides in length.

Embodiment 58: The system of Embodiment 56 or 57, wherein said single-stranded C nucleotide is at position 6, 7, 8, 9, 10, 11, 12, counting from 5′-end of the loop structure.

Embodiment 59: The system of any one of Embodiments 56-58, wherein said loop structure is in form of hairpin and said C nucleotide is present in a single stranded region of the hairpin.

Embodiment 60: The system of any one of Embodiments 56-59, wherein said loop structure is at a position opposite of position 8, 9, 10, 11, 12 or 13, counting from 3′-end or 5′-end, of said strand having a nucleotide sequence substantially complementary to the target RNA (e.g., said loop structure is at a position opposite of position 8, 9, 10, 11, 12 or 13, counting from the 5′-end, of said strand having a nucleotide sequence substantially complementary to the target RNA).

Embodiment 61: The system of any one of Embodiments 56-60, wherein the oligonucleotide comprises at least one nucleic acid modification.

Embodiment 62: The system of any one of Embodiments 56-61, wherein the oligonucleotide comprises at least one nucleic acid modification capable of inhibiting RNA interference cleavage.

Embodiment 63: The kit of Embodiment 40, wherein the kit further comprises a nucleic acid of any one of Embodiments 56-62.

Embodiment 64: The composition of Embodiment 42, wherein the composition further comprises a nucleic acid of any one of Embodiments 56-62.

Embodiment 65: The cell of Embodiment 44, wherein the cell further comprises a nucleic acid of any one of Embodiments 56-62.

Embodiment 66: A method of modifying a target RNA, the method comprising contacting the target RNA with the system of any one of Embodiments 56-62.

Embodiment 67: The method of Embodiment 66, wherein the RNA is an mRNA.

Embodiment 68: The method of Embodiment 66 or 67, wherein the RNA is in a cell.

Embodiment 69: The method of any one of Embodiments 66-68, wherein said contacting is in vitro.

Embodiment 70: The method of any one of Embodiment 66-69, wherein said contacting is in vivo.

Embodiment 71: The method of any one of Embodiments 46-50, wherein said modifying the target RNA comprises deamination of a cytidine in the target RNA.

Embodiment 72: A cell comprising an RNA modified by any one of Embodiments 66-71.

### Definitions

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs. If there is an apparent discrepancy between the usage of a term in the art and its definition provided herein, the definition provided within the specification shall prevail.

For convenience, certain terms employed herein, in the specification, examples and appended claims are collected here.

The terms “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g. the absence of a given treatment or agent) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

The terms “increased”, “increase”, “enhance”, or “activate” are all used herein to mean an increase by a statically significant amount. In some embodiments, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, a “increase” is a statistically significant increase in such level.

As used herein, a “subject” means a human or animal. Usually the animal is a vertebrate such as a primate, rodent, domestic animal, or game animal. Primates include chimpanzees, cynomolgus monkeys, spider monkeys, and macaques, e.g., Rhesus. Rodents include mice, rats, woodchucks, ferrets, rabbits, and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish, and salmon. In some embodiments, the subject is a mammal, e.g., a primate, e.g., a human. The terms, “individual,” “patient” and “subject” are used interchangeably herein.

Preferably, the subject is a mammal. The mammal can be a human, non-human primate, mouse, rat, dog, cat, horse, or cow, but is not limited to these examples. Mammals other than humans can be advantageously used as subjects that represent animal models of a disease or disorder. A subject can be male or female.

A subject can be one who has been previously diagnosed with or identified as suffering from or having a condition in need of treatment. Alternatively, a subject can also be one who has not been previously diagnosed. A “subject in need” of testing for a particular condition can be a subject having that condition, diagnosed as having that condition, or at risk of developing that condition.

By the terms “treat,” “treating” or “treatment of” (and grammatical variations thereof) it is meant that the severity of the subject's condition is reduced, at least partially improved or stabilized and/or that some alleviation, mitigation, decrease or stabilization in at least one clinical symptom is achieved and/or there is a delay in the progression of the disease or disorder.

The terms “prevent,” “preventing” and “prevention” (and grammatical variations thereof) refer to prevention and/or delay of the onset of a disease, disorder and/or a clinical symptom(s) in a subject and/or a reduction in the severity of the onset of the disease, disorder and/or clinical symptom(s) relative to what would occur in the absence of the methods of the invention. The prevention can be complete, e.g., the total absence of the disease, disorder and/or clinical symptom(s). The prevention can also be partial, such that the occurrence of the disease, disorder and/or clinical symptom(s) in the subject and/or the severity of onset is less than what would occur in the absence of the present invention.

As used herein, the terms “protein” and “polypeptide” are used interchangeably to designate a series of amino acid residues, connected to each other by peptide bonds between the alpha-amino and carboxy groups of adjacent residues. The terms “protein”, and “polypeptide” refer to a polymer of amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to a gene product and fragments thereof. Thus, exemplary polypeptides or proteins include gene products, naturally occurring proteins, homologs, orthologs, paralogs, fragments and other equivalents, variants, fragments, and analogs of the foregoing.

The terms “wild-type” or “wt” or “WT” or “native” as used herein is meant an amino acid sequence or a nucleotide sequence that is found in nature, including allelic variations. A wild-type protein, polypeptide, antibody, immunoglobulin, IgG, polynucleotide, DNA, RNA, and the like has an amino acid sequence or a nucleotide sequence that has not been intentionally modified.

In the various embodiments described herein, it is further contemplated that variants (naturally occurring or otherwise), alleles, homologs, conservatively modified variants, and/or conservative substitution variants of any of the particular polypeptides described are encompassed. As to amino acid sequences, one of skill will recognize that individual substitutions, deletions or additions to a nucleic acid, peptide, polypeptide, or protein sequence which alters a single amino acid or a small percentage of amino acids in the encoded sequence is a “conservatively modified variant” where the alteration results in the substitution of an amino acid with a chemically similar amino acid and retains the desired activity of the polypeptide. Such conservatively modified variants are in addition to and do not exclude polymorphic variants, interspecies homologs, and alleles consistent with the disclosure.

The term “amino acid substitution” refers to the replacement of at least one existing amino acid residue in a predetermined or native amino acid sequence with a different “replacement” amino acid. A given amino acid can be replaced by a residue having similar physiochemical characteristics, e.g., substituting one aliphatic residue for another (such as Ile, Val, Leu, or Ala for one another), or substitution of one polar residue for another (such as between Lys and Arg; Glu and Asp; or Gln and Asn). Other such conservative substitutions, e.g., substitutions of entire regions having similar hydrophobicity characteristics, are well known. Polypeptides comprising conservative amino acid substitutions can be tested confirm that a desired activity and specificity of a native or reference polypeptide is retained.

Amino acids can be grouped according to similarities in the properties of their side chains (in A. L. Lehninger, in Biochemistry, second ed., pp. 73-75, Worth Publishers, New York (1975)): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); (4) basic: Lys (K), Arg (R), His (H). Alternatively, naturally occurring residues can be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; (6) aromatic: Trp, Tyr, Phe. Non-conservative substitutions will entail exchanging a member of one of these classes for another class. Particular conservative substitutions include, for example; Ala into Gly or into Ser; Arg into Lys; Asn into Gln or into His; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into Ile or into Leu.

The term “amino acid insertion” refers to the insertion of one or more additional amino acids into a predetermined or native amino acid sequence. The insertion can be one, two, three, four, five, or up to twenty amino acid residues.

The term “amino acid deletion” refers to removal of at least one amino acid from a predetermined or native amino acid sequence. The deletion can be one, two, three, four, five, or up to twenty amino acid residues.

In some embodiments, the polypeptide described herein (or a nucleic acid encoding such a polypeptide) can be a functional fragment of one of the amino acid sequences described herein. As used herein, a “functional fragment” is a fragment or segment of a polypeptide which retains at least 50% of the wild-type reference polypeptide's activity according to the assays described herein. A functional fragment can comprise conservative substitutions of the sequences disclosed herein.

In some embodiments, the polypeptide described herein can be a variant of a sequence described herein. In some embodiments, the variant is a conservatively modified variant. Conservative substitution variants can be obtained by mutations of native nucleotide sequences, for example. A “variant,” as referred to herein, is a polypeptide substantially homologous or orthologous to a native or reference polypeptide, but which has an amino acid sequence different from that of the native or reference polypeptide because of one or a plurality of deletions, insertions, or substitutions. Variant polypeptide-encoding DNA sequences encompass sequences that comprise one or more additions, deletions, or substitutions of nucleotides when compared to a native or reference DNA sequence, but that encode a variant protein or fragment thereof that retains activity. A wide variety of PCR-based site-specific mutagenesis approaches are known in the art and can be applied by the ordinarily skilled artisan to generate and test artificial variants.

The term “nucleic acid” refers to a deoxyribonucleotide or ribonucleotide and polymers thereof in either single strand or double strand form. The term “nucleic acid” is used interchangeably with gene, nucleotide, polynucleotide, cDNA, DNA, and mRNA. The polynucleotides can be in the form of RNA or DNA. Polynucleotides in the form of DNA, cDNA, genomic DNA, nucleic acid analogs, and synthetic DNA are within the scope of the present invention. Unless specifically limited the term encompasses nucleic acids containing known analogues of natural nucleotides that have similar binding propertied as the natural nucleic acid. Unless specifically limited, a particular nucleotide sequence also encompasses conservatively modified variants thereof (for example, those containing degenerate codon substitutions) and complementary sequences as well as the as well as the sequences specifically described.

The polynucleotides can be composed of any polyribonucleotide or polydeoxyribonucleotide, which can be unmodified RNA or DNA or modified RNA or DNA. For example, polynucleotides can be composed of single or double stranded regions, mixed single or double stranded regions. In addition, the polynucleotides can be triple stranded regions containing RNA or DNA or both RNA and DNA. Modified polynucleotides include modified bases, such as tritylated bases or unusual bases such as inosine. A variety of modification can be made to RNA and DNA; thus, polynucleotide includes chemically, enzymatically, or metabolically modified forms.

The DNA may be double-stranded or single-stranded, and if single stranded, may be the coding (sense) strand or non-coding (anti-sense) strand. The coding sequence that encodes the polypeptide may be identical to the coding sequence provided herein or may be a different coding sequence, which sequence, as a result of the redundancy or degeneracy of the genetic code, encodes the same polypeptides as the DNA provided herein.

A variant DNA or amino acid sequence can be at least 90%, at least 91%, at least 92%, at least 93%, at least 94%, at least 95%, at least 96%, at least 97%, at least 98%, at least 99%, or more, identical to a native or reference sequence. The degree of homology (percent identity) between a native and a mutant sequence can be determined, for example, by comparing the two sequences using freely available computer programs commonly employed for this purpose on the world wide web (e.g., BLASTp or BLASTn with default settings).

In some embodiments of the various aspects described herein, a polypeptide, nucleic acid, or cell as described herein can be engineered. As used herein, “engineered” refers to the aspect of having been manipulated by the hand of man. For example, a polynucleotide is considered to be “engineered” when at least one aspect of the polynucleotide, e.g., its sequence, has been manipulated by the hand of man to differ from the aspect as it exists in nature.

As used herein, the term “specific binding” refers to a chemical interaction between two molecules, compounds, cells and/or particles wherein the first entity binds to the second, target entity with greater specificity and affinity than it binds to a third entity which is a non-target. In some embodiments, specific binding can refer to an affinity of the first entity for the second target entity which is at least 10 times, at least 50 times, at least 100 times, at least 500 times, at least 1000 times or greater than the affinity for the third non-target entity. A reagent specific for a given target is one that exhibits specific binding for that target under the conditions of the assay being utilized.

The term “statistically significant” or “significantly” refers to statistical significance and generally means a two standard deviation (2SD) or greater difference.

Other than in the operating examples, or where otherwise indicated, all numbers expressing quantities of ingredients or reaction conditions used herein should be understood as modified in all instances by the term “about.” The term “about” when used in connection with percentages can mean 10. In some embodiments of the various aspects described herein, the term “about” when used in connection with percentages can mean±5%.

As used herein, the term “comprising” means that other elements can also be present in addition to the defined elements presented. The use of “comprising” indicates inclusion rather than limitation.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the invention.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Unless otherwise defined herein, scientific, and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in immunology and molecular biology can be found in The Merck Manual of Diagnosis and Therapy, 20th Edition, published by Merck Sharp & Dohme Corp., 2018 (ISBN 0911910190, 978-0911910421); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), W. W. Norton & Company, 2016 (ISBN 0815345054, 978-0815345053); Lewin's Genes XI, published by Jones & Bartlett Publishers, 2014 (ISBN−1449659055); Michael Richard Green and Joseph Sambrook, Molecular Cloning: A Laboratory Manual, 4th ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

Other terms are defined herein within the description of the various aspects of the invention.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The technology described herein is further illustrated by the following examples which in no way should be construed as being further limiting.

### EXAMPLES

### Example 1. RNA Editing by ADAR-hAgo2

### Reagents:

1. Plasmid:
   pAAV-Basic-eGFP-WT (“WT-eGFP”);
   pAAV-Basic-eGFP-W58X (“eGFP-W58X”);
   pCMV-3tag-6 (“empty vector”);
   pCMV-3tag-6-Flag-ADARDDE448Q-4GS1-Nes-hAgo2-D597A/D669A (“ADAR-hAgo2”);
2. Editing siRNA
3. Transfection reagents: Lipofectamine™ 3000, or Lipofectamine™ RNAiMax (Invitrogen/Thermo Fisher Scientific).
4. Cell line: Hek293

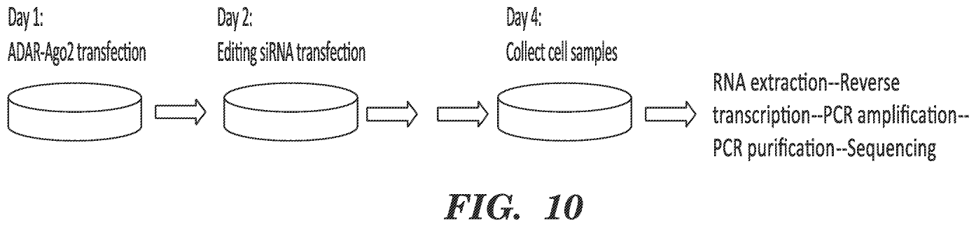

The editing protocol herein is exemplified by FIG. 10. Therein, on day 1, HEK293 cells were seeded to a 12-well plate at approximate 0.5 million per-well, to target 70% confluence for transfection in day 2. Cell were transfected with Lipo3000 according to the supplier protocol using either 0.3 μg eGFP-W58X and 0.7 μg ADAR-hAgo2 per-well, 0.3 μg WT-eGFP and 0.7 ADAR-hAgo2 per well (positive control), or 0.3 μg eGFP-W58X and 0.7 μg empty vector (negative control). The transfected cells were maintained overnight. On day 3, cell medium was replaced with fresh medium, avoiding cell detachment.

Six hours after changing the cell medium, editing siRNA (20 nM per well) was transfected with RNAiMax following supplier protocol. Medium was subsequently changed 24 hours after siRNA transfection and cells imaged by fluorescence microscopy. Cells were imaged again by fluorescence microscopy at 48 hours after siRNA transfection. Cell samples were collected no later than 60 hours after siRNA transfection for RNA extraction. RNA was extracted either immediately or cell pellets were stored at −80° C. until extraction.

Total RNA was extracted with The Invitrogen PureLink RNA Mini Kit or Qiagen Kit followed by reverse transcription with Maxima H Minus First Strand cDNA Synthesis Kit (K1651). The amount of total RNA for reverse transcription was 500 ng. A gene specific primer with 5′ adapter (underline) was used; for eGFP the primer was eGFP-CDNA:

(SEQ ID NO: 83)
CTGATCTAGAGGTACCGGATCA GTGTCGCCCTCGAACTTCAC

The reverse transcription product was amplified by PCR with Primerstar max (Takara, R045A) following the supplier protocol using a template cDNA (2 μL) for total 40 μL PCR reaction. Forward primer was the eGFP sequence and reverse primer was a 5′ adapter of gene specific primer for RT. The PCR product (approximately 200 kb) was loaded to 1% agarose and the gel was run for 30 min at 150 V. The resulting gel fragment containing DNA was excised and purified with Macherey-Nagel kit (REF 74060) to provide purified DNA that was sequenced via Sanger Sequencing.

### Example 2. Positional Dependence of RNA Editing with ADAR-hAgo2

Using the editing protocol of Example 1, the positional dependence of the C-A mismatch between the eGFP-W58X reporter mRNA and the antisense strand of the editing siRNA was examined using the siRNA in Table 1, where the location of the C-A mismatch is indicated by “MM mismatch” and the corresponding cytosine is bolded. Editing siRNAs 1-7 were fully unmodified RNA having the indicated antisense sequence (replacing any T for U) and a corresponding sense strand of 19 unmodified ribonucleotides in length and fully matched to position 1-19 of the antisense strand (leaving a 3′-overhang of 2 nucleotides). Editing siRNAs 8-10 were fully unmodified RNA having the indicated antisense sequence (replacing any T for U) and a corresponding sense strand of 23 unmodified ribonucleotides in length and fully matched to position 1-23 of the antisense strand (leaving a 3′-overhang of 2 nucleotides). Editing siRNAs 11 and 12 were fully unmodified RNA having the indicated antisense sequence (replacing any T for U) and a corresponding sense strand of 25 unmodified ribonucleotides in length and fully matched to position 1-25 of the antisense strand (leaving a 3′-overhang of 2 nucleotides).

TABLE 1

| siRNA | | | MM | SEQ ID |
|---|---|---|---|---|
| # | Strand | Sequence | position | NO. |
| 1 | Antisense | 5′-P-GTGGG CCAGG GCACG GGCAG C-3′ | 7 | 84 |
| 2 | Antisense | 5′-P-GGGTG GGCCA GGGCA CGGGC A-3′ | 9 | 85 |
| 3 | Antisense | 5′-P-GAGGG TGGGC CAGGG CACGG G-3′ | 11 | 86 |
| 4 | Antisense | 5′-P-ACGAG GGTGG GCCAG GGCAC G-3′ | 13 | 87 |
| 5 | Antisense | 5′-P-TCACG AGGGT GGGCC AGGGC A-3′ | 15 | 88 |
| 6 | Antisense | 5′-P-GGTCA CGAGG GTGGG CCAGG G-3′ | 17 | 89 |
| 7 | Antisense | 5′-P-GTGGT CACGA GGGTG GGCCA G-3′ | 19 | 90 |
| 8 | Antisense | 5′-P-GGGTG GTCAC GAGGG TGGGC | 21 | 91 |
| | | CAGGG-3′ | | |
| 9 | Antisense | 5′-P-AGGGT GGTCA CGAGG GTGGG | 22 | 92 |
| | | CCAGG-3′ | | |
| 10 | Antisense | 5′-P-CAGGG TGGTC ACGAG GGTGG | 23 | 93 |
| | | GCCAG-3′ | | |
| 11 | Antisense | 5′-P-UCAGG GTGGT CACGA GGGTG | 24 | 94 |
| | | GGCCA GG-3′ | | |
| 12 | Antisense | 5′-P-GUCAG GGTGG TCACG AGGGT | 25 | 95 |
| | | GGGCC AG-3′ | | |

where P is a 5′-phosphate

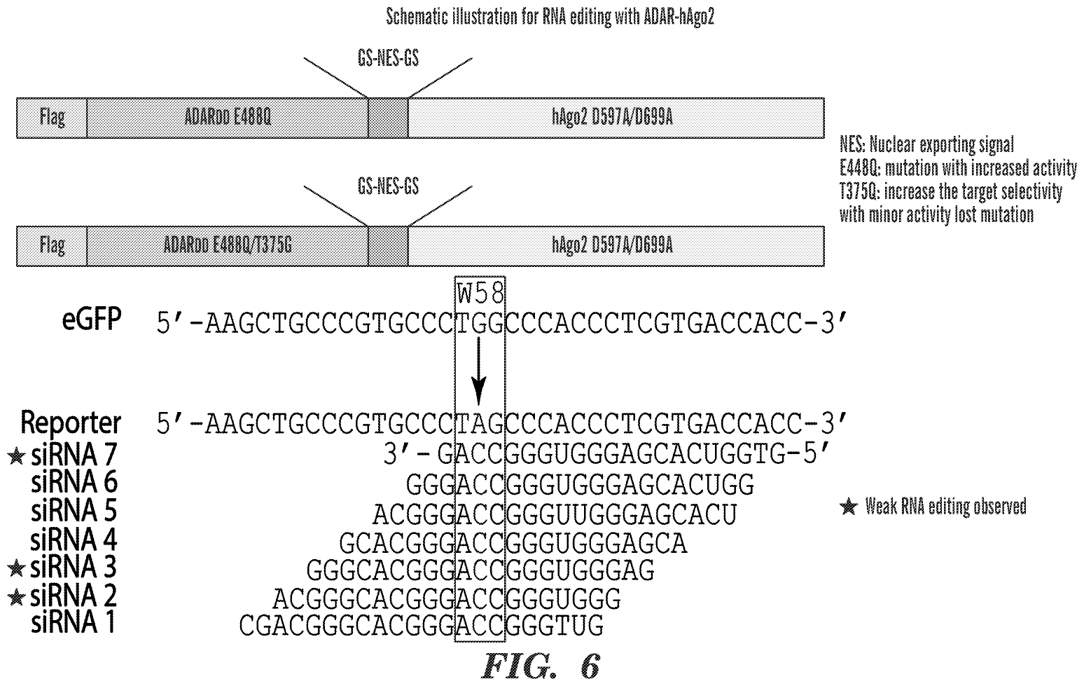

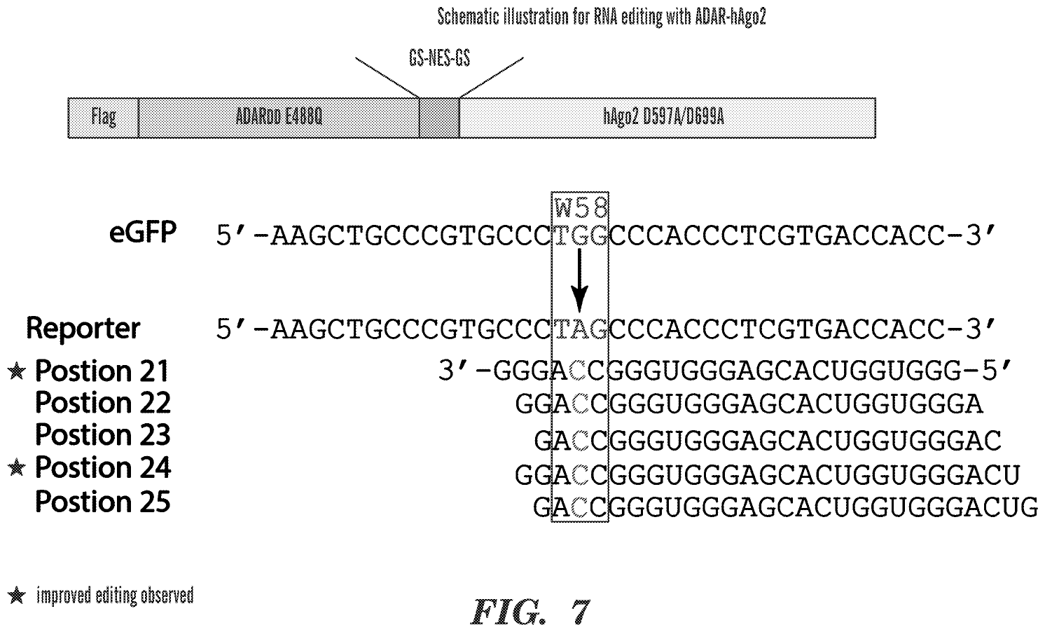

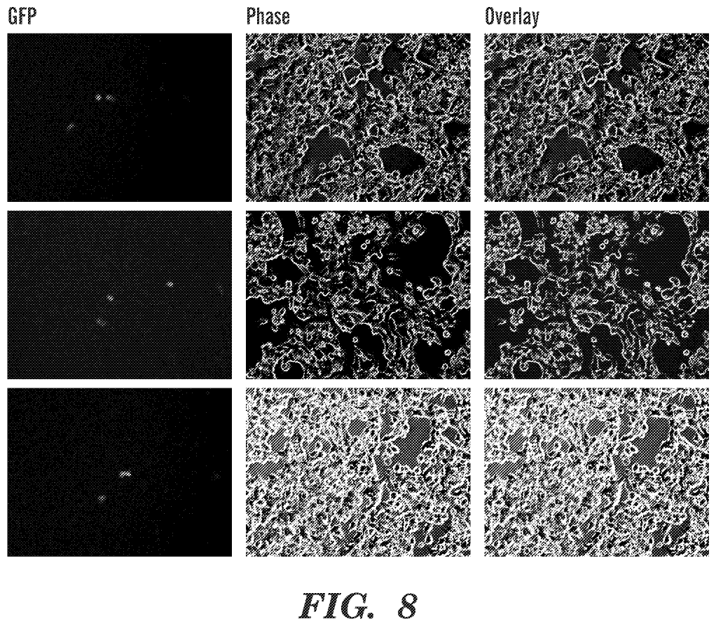

As indicated in FIG. 6, weak RNA editing was observed using editing siRNA-2, siRNA-3, and siRNA-7, having the C-A mismatch as antisense positions 9, 11, and 19, counting from the 5′-end of the antisense strand. FIG. 8 shows the RNA editing achieved with editing siRNA 7 as illustrated by cells having increased green fluorescence. Extending the length of the siRNA design, as shown in FIG. 7, provided improved editing with editing siRNA-8 (position 21) and siRNA-11 (position 24).

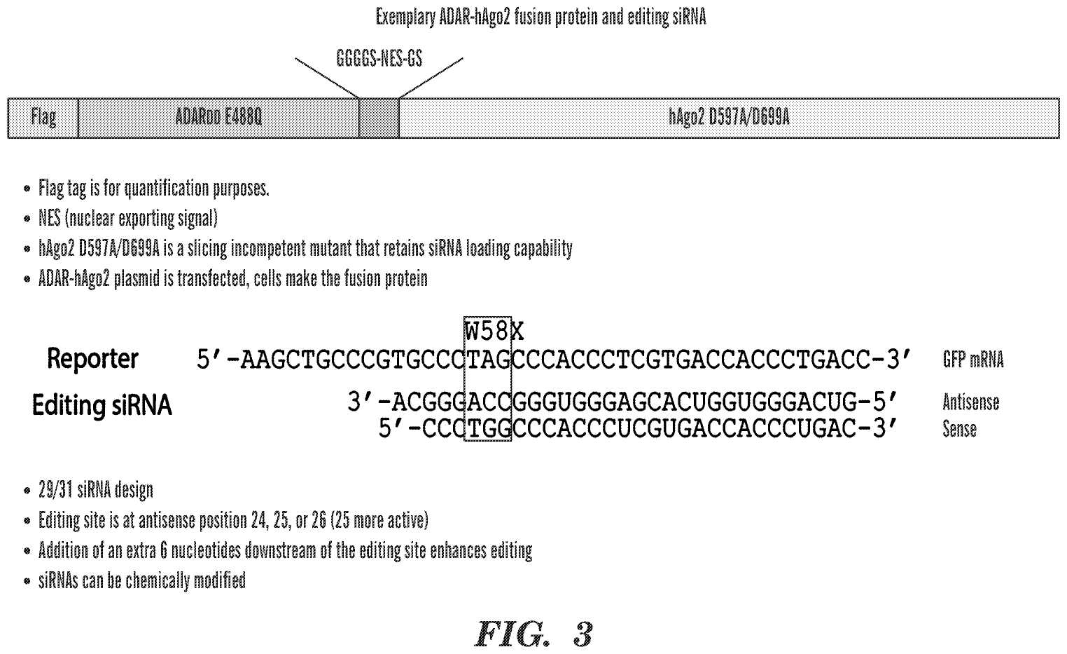

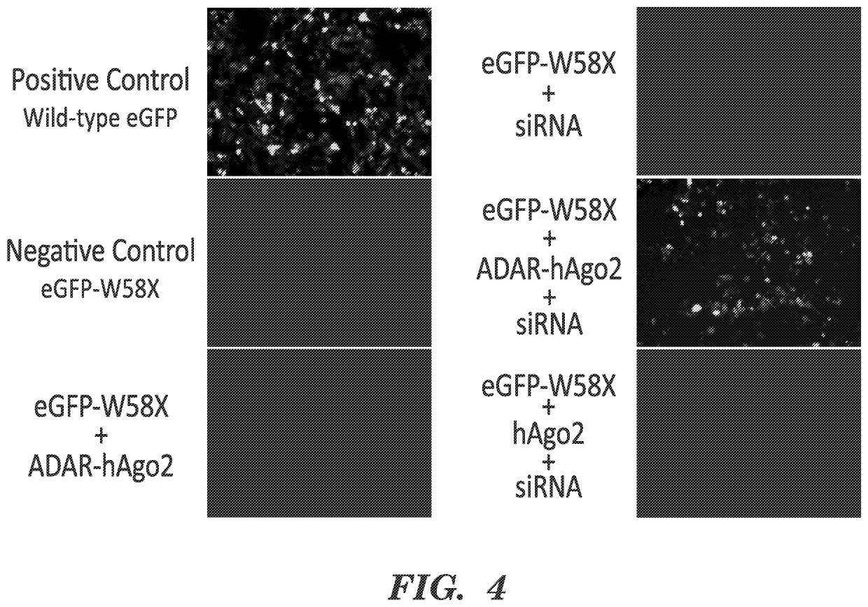

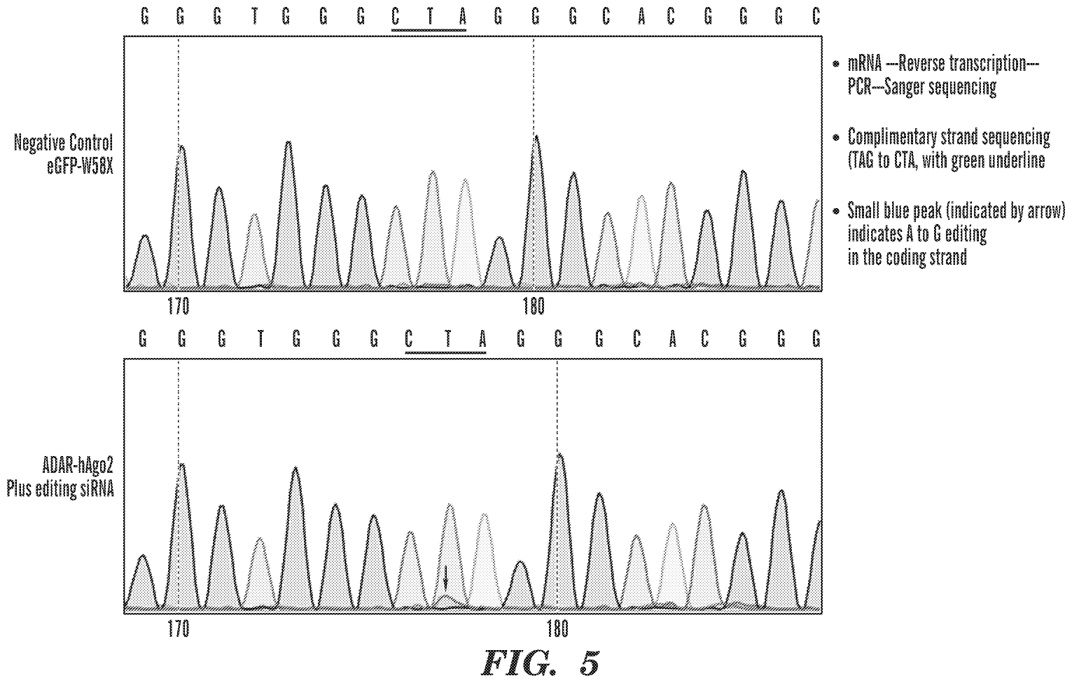

As shown in FIG. 4, induction of editing siRNA-13 (see below and FIG. 3) to cells transfected with both eGFP-W58X and ADAR-hAgo2 restored translation of eGFP as shown by turn-on of fluorescence. Site specific editing of A to I was confirmed by Sanger Sequencing of the complementary strand as shown in FIG. 5, where a peak indicated A to G editing in the coding strand.

TABLE 2

| siRNA | | | SEQ |
|---|---|---|---|
| # | Strand | Sequence | ID NO. |
| 13 | Sense | 5′-CCCUG GCCCA CCCUC GUGAC CACCC UGAC-3′ | 96 |
| | Antisense | 5′-P-GUCAG GGUGG UCACG AGGGU GGGCC AGGGC A-3′ | 97 |

where P is a 5′-phosphate.

### Example 3. RNA Editing by Modified ADAR-hAgo Fusion Proteins

Changes in the ADAR-hAgo2 fusion polypeptide of Example 1 were examined for effect on editing activity. Using the protocol of Example 1 and the editing siRNA-13 (FIG. 3) having a 29/31 S/AS design, substituting either hAgo2 with hAgo2-ΔN51, hAgo1, hAgo3, or hAgo4; or substituting the GGGS-NES-GS linker in ADAR-hAgo2 with a GGSGGGSSGAAAGSGG (SEQ ID NO: 77) linker were each examined.

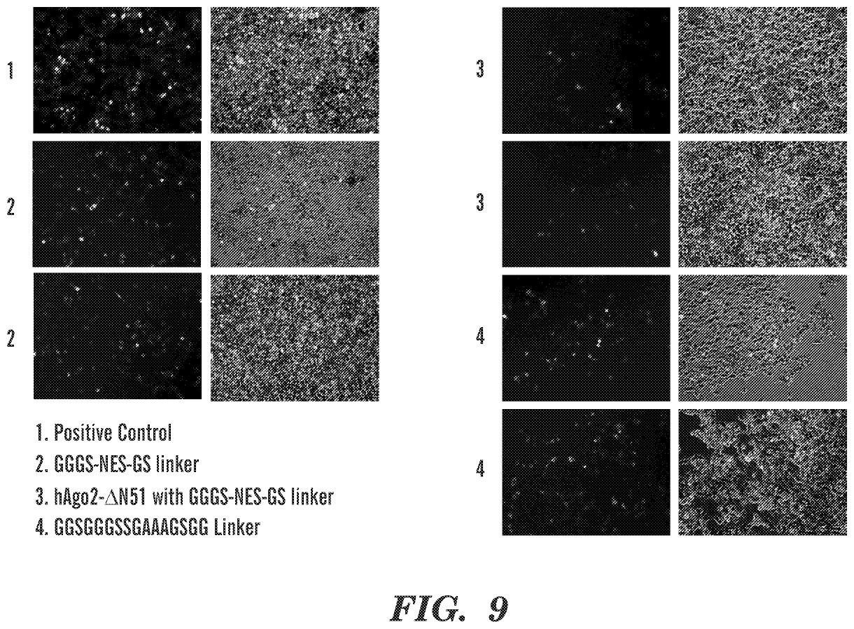

FIG. 9 shows imaging results for (1) WT-eGFP positive control; (2) ADAR-hAgo2; (3) hAgo2-ΔN51 substitution; and (4) GGSGGGSSGAAAGSGG (SEQ ID NO: 77) linker substitution. While linker substitution was tolerated. hAgo2-ΔN51 substitution provided reduced editing activity, indicating that the N-terminal amino acids 1˜51 of hAgo2 may be important for either siRNA loading or target recognition.

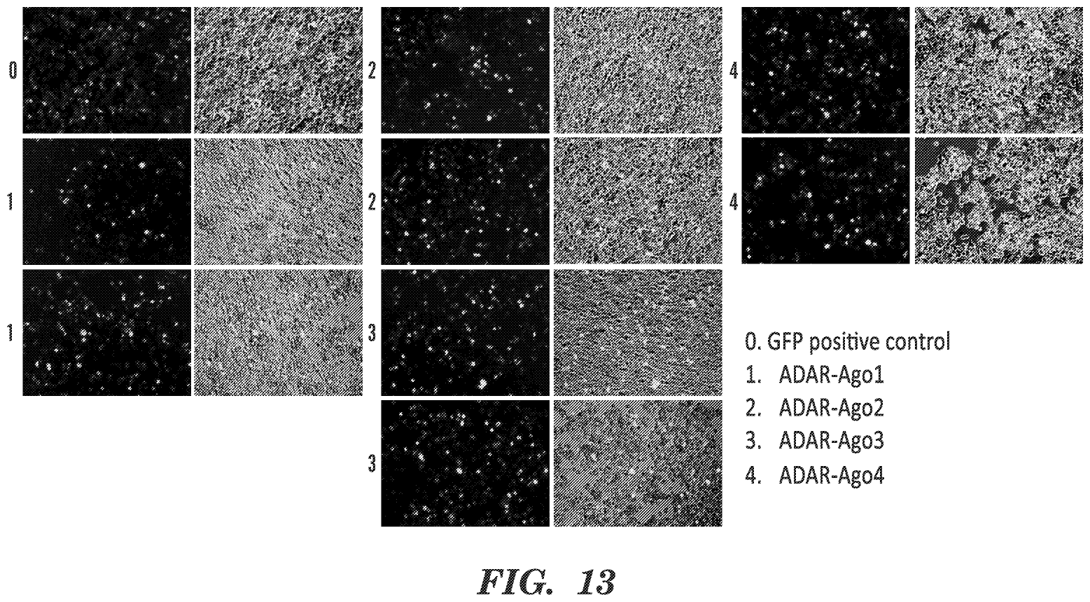

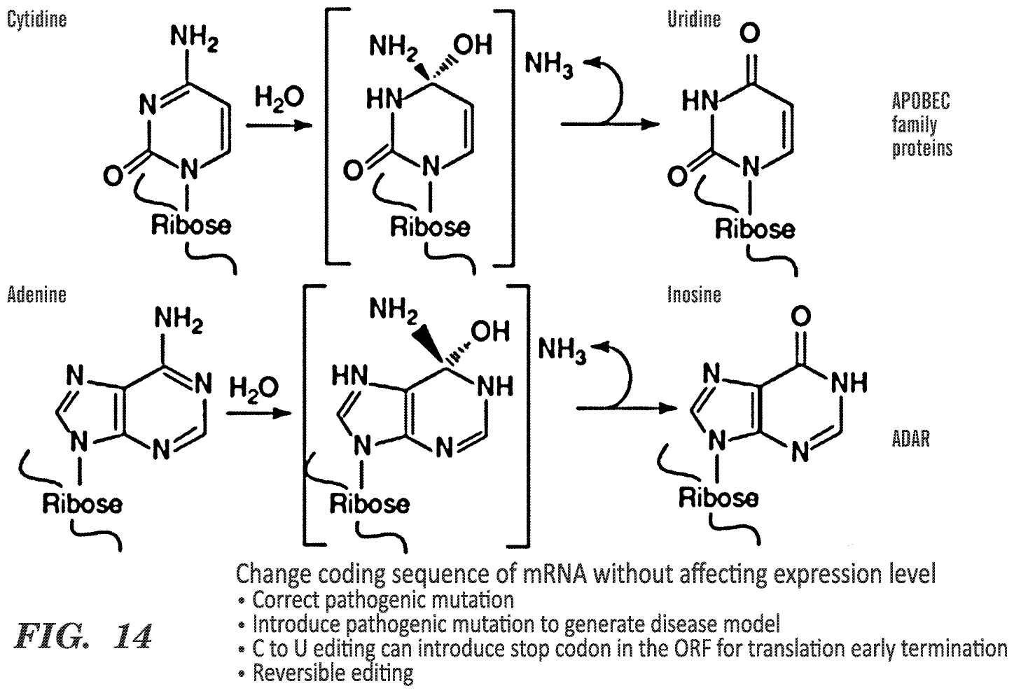

FIG. 13 shows imaging results for (0) WT-eGFP positive control; (1) ADAR-hAgo1; (2) ADAR-hAgo2; (3) ADAR-hAgo3; and (4) ADAR-hAgo4. Therein, RNA editing was seen with each of ADAR-Ago1-4, illustrating that all human Ago proteins work in the fusion construct.

### Example 4. RNA Editing of Endogenous Leucyl-tRNA Synthetase 1 (LARS1/LeuRS) and WD Repeat Domain 5 (WDR5) mRNA

Editing siRNA were designed for site specific editing of endogenous target mRNA of two human genes (LARS1 and WDR5), and contained a mismatch to the target sequence at antisense position 10 (AS10) to block slicer activity. Table 3 provides the editing site (where the targeted adenosine is bolded along with the fully unmodified ribonucleotide editing siRNA designed to that target. Each target mRNA site is noted in Table 3 as the corresponding genetic sequence and the targeted mRNA has U substituted for each T.

TABLE 3
Leucyl-tRNA synthetase 1 (LARS1/LeuRS)

| | | SEQ ID |
|---|---|---|
| | Sequence | NO. |
| Target | 5′-TGTACCCAAAAAGCAGCCAGGAATATGTCATAC | 98 |
| mRNA | CAGGGCTTT-3′ | |
| site: | | |
| Editing | Sense: 5′-AAAAG GCAGC CAGGA AUAUC | 99 |
| | UCAUA CCAA-3′ | |
| siRNA: | Antisense:3′-G GUUUU CCGUC GGUCC UUAUA | 100 |
| | GAGUA UGGUU-P-5′ | |

WD repeat domain 5 (WDR5)

| | | |
|---|---|---|
| Target | 5′-ATAGTTTCAAGTAGCTATGATGGTCTCTG | 101 |
| mRNA | TCGCATCTGGG-3′ | |
| site: | | |
| Editing | Sense: 5′-AAGUG GCUAU GAUGG UCUCC | 102 |
| siRNA: | GUCGC AUCA-3′ | |
| | Antisense: 3′-A GUUCA CCGAU ACUAC CAGAG GCAGC | 103 |
| | GUAGU-P-5′ | |

where P is a 5′-phosphate.

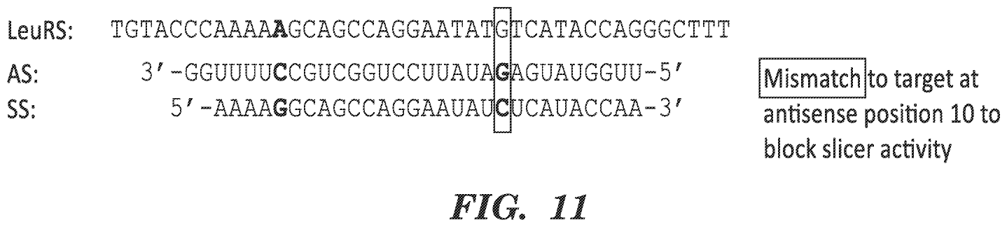

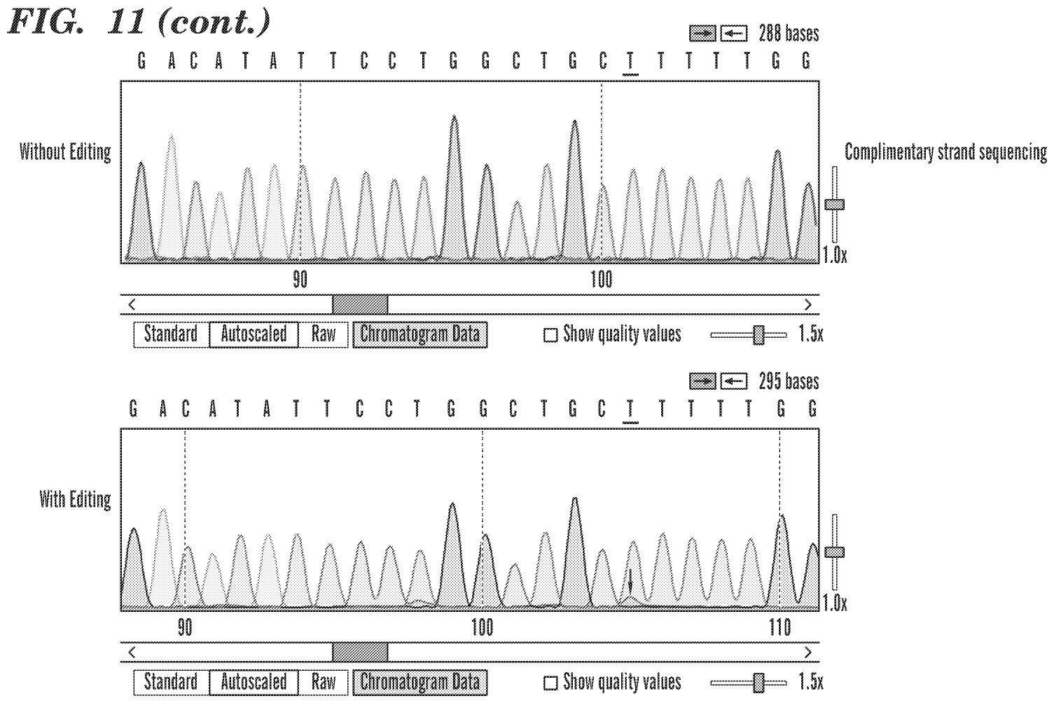

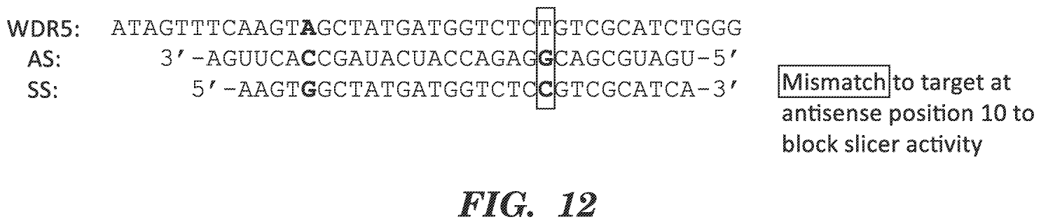

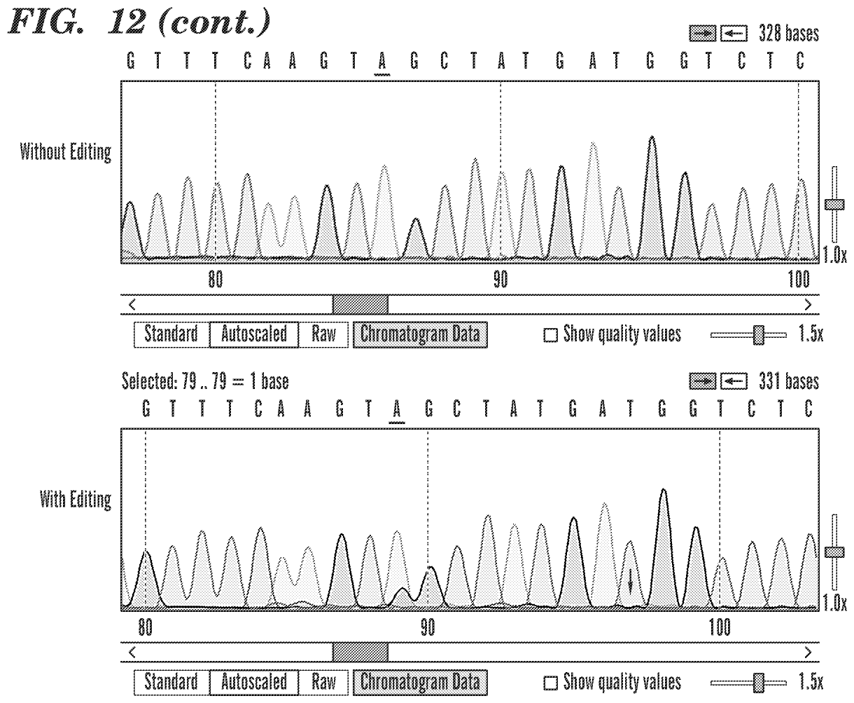

RNA editing was performed with ADAR-Ago2 and RNA isolated for Sanger Sequencing, each according to Example 1, omitting any GFP transfection, and substituting the preceding editing siRNA and PCR forward primers for the respective gene sequence. FIGS. 11 and 12 illustrate successful RNA editing as seen in Sanger Sequencing of the isolated coding strand for LARS1 and WDR5, respectively.

### Example 5. Endogenous RNA Editing by APOBEC3A-hAgo2

APOBEC3A (Y132D or Y132R) was fused through a GGGS-NES-GS linker to catalytic inactive Ago2 via expression of pCMV-APOBEC3A-Y132R-4GS1-NES-Ago2-D597A_D669A (“APOBEC3A-Y132R”) or pCMV-APOBEC3A-Y132D-4GS1-NES-Ago2-D597A/D669A (“APOBEC3A-Y132D”). Editing siRNA were designed for site specific C→U editing at a cytosine within a 14 nucleotide loop introduced in a target sequence of WDR5 when complexed to the antisense sequence of editing siRNA. The loop was located between antisense positions 10 and 11, as shown in FIG. 15. The editing siRNAs was a fully unmodified RNA having the indicated antisense sequence of Table 3 and a corresponding sense strand of 21 unmodified ribonucleotides in length and fully matched to position 1-21 of the antisense strand (leaving a 3′-overhang of 2 nucleotides). The loop region is underlined, and the targeted cytosine bolded in Table 4.

TABLE 4
WD repeat domain 5 (WDR5)

| | | |
|---|---|---|
| Target | 5′-UCGGAUCCAGUCUCGGCCGUUCAUUUU | SEQ ID NO. 104 |
| mRNA site: | AAUCGUGAUGGAUCCUUGAUAGUUUC-3′ | |
| Editing | Antisense: 5′-P-UCAAG GAUCC GAACG GCCGA | SEQ ID NO. 105 |
| siRNA: | GAC-3′ | |

where P is a 5′-phosphate.

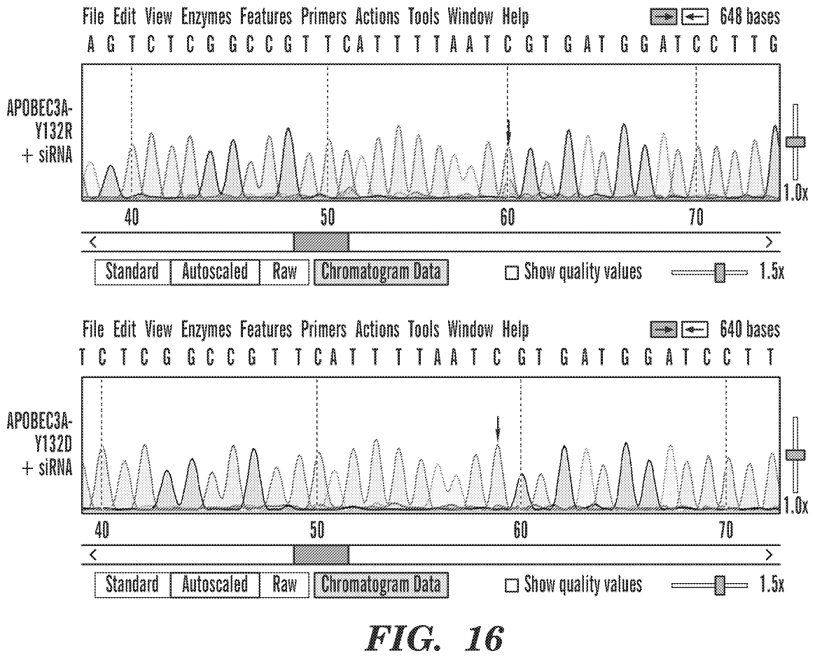

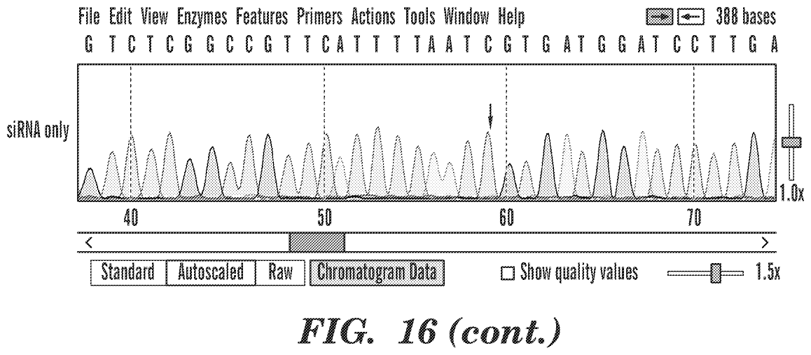

RNA editing was performed according to Example 1, omitting any GFP transfection, substituting APOBEC3A-Y132R or APOBEC3A-Y132D for ADAR-hAgo2, and using the editing siRNA of Table 4. A control experiment transfected the editing siRNA in the absence of either fusion protein. RNA was isolated for Sanger Sequencing according to Example 1, a PCR forward primer for the WDR5 gene sequence. FIG. 16 illustrates successful RNA editing in the presence of the APOBEC3A-Y132R fusion protein as seen in Sanger Sequencing of the complementary strand WDR5 (top) as compared to APOBEC3A-Y132D (middle) or control (bottom).

### Sequences of Polynucleotides Encoding Exemplary Polypeptides

FLAG-ADAR-GGGGS-NES-hAgo1
(SEQ ID NO: 106)
ATG GACTACAAAGACGATGACGACAAG GCGGCCGCA
Cagctgcatttaccgcaggttttagctgacgctgtctcacgcctggtcctgggtaagtttggtgacctgaccgacaacttctcctcccctcacg
ctcgcagaaaagtgctggctggagtcgtcatgacaacaggcacagatgttaaagatgccaaggtgataagtgtttctacaggaacaaaatgt
attaatggtgaatacatgagtgatcgtggccttgcattaaatgactgccatgcagaaataatatctcggagatccttgctcagatttctttatac
acaacttgagctttacttaaataacaaagatgatcaaaaaagatccatctttcagaaatcagagcgaggggggtttaggctgaaggagaatgtcc
agtttcatctgtacatcagcacctctccctgtggagatgccagaatcttctcaccacatgagccaatcctggaagaaccagcagatagacacc
caaatcgtaaagcaagaggacagctacggaccaaaatagagtctggtCaggggacgattccagtgcgctccaatgcgagcatccaaacgt
gggacggggtgctgcaaggggagcggctgctcaccatgtcctgcagtgacaagattgcacgctggaacgtggtgggcatccagggatc
Actgctcagcattttcgtggagcccatttacttctcgagcatcatcctgggcagcctttaccacggggaccacctttccagggccatgtaccag
cggatctccaacatagaggacctgccacctctctacaccctcaacaagcctttgctcagtggcatcagcaatgcagaagcacggcagccag
ggaaggcccccaacttcagtgtcaactggacggtaggcgactccgctattgaggtcatcaacgccacgactgggaaggatgagctgggcc
gcgcgtcccgcctgtgtaagcacgcgttgtactgtcgctggatgcgtgtgcacggcaaggttccctcccacttactacgctccaagattacca
agcccaacgtgtaccatgagtccaagctggcggcaaaggagtaccaggccgccaaggcgcgtctgttcacagccttcatcaaggcgggg
ctgggggcctgggtggagaagcccaccgagcaggaccagttctcactcacg
GGAGGTGGCGGTAGTCTGCAGCTGCCTCCACTTGAAAGACTGACACTGGGATCC
gaagcgggaccctcgggagcagctgcgggcgcttacctgccccccctgcagcaggtgttccaggcacctcgccggcctggcattggcac
tgtggggaaaccaatcaagctcctggccaattactttgaggtggacatccctaagatcgacgtgtaccactacgaggtggacatcaagccgg
ataagtgtccccgtagagtcaaccgggaagtggtggaatacatggtccagcatttcaagcctcagatctttggtgatcgcaagcctgtgtatga
tggaaagaagaacatttacactgtcacagcactgcccattggcaacgaacgggtcgactttgaggtgacaatccctggggaagggaaggat
cgaatctttaaggtctccatcaagtggctagccattgtgagctggcgaatgctgcatgaggccctggtcagcggccagatccctgttcccttg
gagtctgtgcaagccctggatgtggccatgaggcacctggcatccatgaggtacacccctgtgggccgctccttcttctcaccgcctgaggg
ctactaccacccgctggggggtgggcgcgaggtctggttcggctttcaccagtctgtgcgccctgccatgtggaagatgatgctcaacattg
atgtctcagccactgccttttataaggcacagccagtgattgagttcatgtgtgaggtgctggacatcaggaacatagatgagcagcccaagc
ccctcacggactctcagcgcgttcgcttcaccaaggagatcaagggcctgaaggtggaagtcacccactgtggacagatgaagaggaagt
accgcgtgtgtaatgttacccgtcgccctgctagccatcagacattccccttacagctggagagtggacagactgtggagtgcacagtggca
cagtatttcaagcagaaatataaccttcagctcaagtatccccatctgccctgcctacaagttggccaggaacaaaagcatacctaccttcccct
agaggtctgtaacattgtggctgggcagcgctgtattaaaaagctgaccgacaaccagacctcgaccatgataaaggccacagctagatcc
gctccagacagacaggaggagatcagtcgcctgatgaagaatgccagctacaacttagatccctacatccaggaatttgggatcaaagtga
aggatgacatgacggaggtgacagggcgagtgctgccggcgcccatcttgcagtacggcggccggaaccgggccattgccacacccaa
tcagggtgtctgggacatgcgggggaaacagttctacaatgggattgagatcaaagtctgggccatcgcctgcttcgcaccccaaaaacagt
gtcgagaagaggtgctcaagaacttcacagaccagctgcggaagatttccaaggatgcggggatgcctatccagggtcaaccttgtttctgc
aaatatgcacagggggcagacagcgtggagcctatgttccggcatctcaagaacacctactcagggctgcagctcattattgtcatcctgcc
agggaagacgccggtgtatgctgaggtgaaacgtgtcggagatacactcttgggaatggctacgcagtgtgtgcaggtgaagaacgtggtc
aagacctcacctcagactctgtccaacctctgcctcaagatcaatgtcaaacttggtggcattaacaacatcctagtcccacaccagcgctctg
ccgtttttcaacagccagtgatattcctgggagcagatgttacacaccccccagcaggggatgggaaaaaaccttctatcacagcagtggta
ggcagtatggatgcccaccccagccgatactgtgctactgtgcgggtacagcgaccacggcaagagatcattgaagacttgtcctacatggt
gcgtgagctcctcatccaattctacaagtccacccgtttcaagcctacccgcatcatcttctaccgagatggggtgcctgaaggccagctacc
ccagatactccactatgagctactggccattcgtgatgcctgcatcaaactggaaaaggactaccagcctgggatcacttatattgtggtgcag
aaacgccatcacacccgccttttctgtgctgacaagaatgagcgaattgggaagagtggtaacatcccagctgggaccacagtggacacca
acatcacccacccatttgagtttgacttctatctgtgcagccacgcaggcatccagggcaccagccgaccatcccattactatgttctttgggat
gacaaccgtttcacagcagatgagctccagatcctgacgtaccagctgtgccacacttacgtacgatgcacacgctctgtctctatcccagca
cctgcctactatgcccgcctggtggctttccgggcacgataccacctggtggacaaggagcatgacagtggagaggggagccacatatcg
gggcagagcaatgggcgggacccccaggccctggccaaagccgtgcaggttcaccaggatactctgcgcaccatgtacttcgcttga
ADAR-4GS-1-hAgo2-D597A/D669A
(SEQ ID NO: 107)
ATGGACTACAAAGACGATGACGACAAGGCGGCCGCAcagctgcatttaccgcaggttttagctgacgctg
tctcacgcctggtcctgggtaagtttggtgacctgaccgacaacttctcctcccctcacgctcgcagaaaagtgctggctggagtcgtcatga
caacaggcacagatgttaaagatgccaaggtgataagtgtttctacaggaGGCaaatgtattaatggtgaatacatgagtgatcgtggcctt
gcattaaatgactgccatgcagaaataatatctcggagatccttgctcagatttctttatacacaacttgagctttacttaaataacaaagatga
tcaaaaaagatccatctttcagaaatcagagcgaggggggtttaggctgaaggagaatgtccagtttcatctgtacatcagcacctctccctgtg
gagatgccagaatcttctcaccacatgagccaatcctggaagaaccagcagatagacacccaaatcgtaaagcaagaggacagctacgga
ccaaaatagagtctggtCaggggacgattccagtgcgctccaatgcgagcatccaaacgtgggacggggtgctgcaaggggagcggct
gctcaccatgtcctgcagtgacaagattgcacgctggaacgtggtgggcatccagggatcActgctcagcattttcgtggagcccatttactt
ctcgagcatcatcctgggcagcctttaccacggggaccacctttccagggccatgtaccagcggatctccaacatagaggacctgccacctc
tctacaccctcaacaagcctttgctcagtggcatcagcaatgcagaagcacggcagccagggaaggcccccaacttcagtgtcaactggac
ggtaggcgactccgctattgaggtcatcaacgccacgactgggaaggatgagctgggccgcgcgtcccgcctgtgtaagcacgcgttgta
ctgtcgctggatgcgtgtgcacggcaaggttccctcccacttactacgctccaagattaccaagcccaacgtgtaccatgagtccaagctggc
ggcaaaggagtaccaggccgccaaggcgcgtctgttcacagccttcatcaaggcggggctgggggcctgggtggagaagcccaccgag
caggaccagttctcactcacg
GGAGGTGGCGGTAGTCTGCAGCTGCCTCCACTTGAAAGACTGACACTGGGATCC
TACTCGGGAGCCGGCCCCGCACTTGCACCTCCTGCGCCGCCGCCCCCCATCCAAGGA
TATGCCTTCAAGCCTCCACCTAGACCCGACTTTGGGACCTCCGGGAGAACAATCAAA
TTACAGGCCAATTTCTTCGAAATGGACATCCCCAAAATTGACATCTATCATTATGAAT
TGGATATCAAGCCAGAGAAGTGCCCGAGGAGAGTTAACAGGGAAATCGTGGAACAC
ATGGTCCAGCACTTTAAAACACAGATCTTTGGGGATCGGAAGCCCGTGTTTGACGGC
AGGAAGAATCTATACACAGCCATGCCCCTTCCGATTGGGAGGGACAAGGTGGAGCT
GGAGGTCACGCTGCCAGGAGAAGGCAAGGATCGCATCTTCAAGGTGTCCATCAAGT
GGGTGTCCTGCGTGAGCTTGCAGGCGTTACACGATGCACTTTCAGGGCGGCTGCCCA
GCGTCCCTTTTGAGACGATCCAGGCCCTGGACGTGGTCATGAGGCACTTGCCATCCA
TGAGGTACACCCCCGTGGGCCGCTCCTTCTTCACCGCGTCCGAAGGCTGCTCTAACCC
TCTTGGCGGGGGCCGAGAAGTGTGGTTTGGCTTCCATCAGTCCGTCCGGCCTTCTCTC
TGGAAAATGATGCTGAATATTGATGTGTCAGCAACAGCGTTTTACAAGGCACAGCCA
GTAATCGAGTTTGTTTGTGAAGTTTTGGATTTTAAAAGTATTGAAGAACAACAAAAA
CCTCTGACAGATTCCCAAAGGGTAAAGTTTACCAAAGAAATTAAAGGTCTAAAGGTG
GAGATAACGCACTGTGGGCAGATGAAGAGGAAGTACCGCGTCTGCAATGTGACCCG
GCGGCCCGCCAGTCACCAAACATTCCCGCTGCAGCAGGAGAGCGGGCAGACGGTGG
AGTGCACGGTGGCCCAGTATTTCAAGGACAGGCACAAGTTGGTTCTGCGCTACCCCC
ACCTCCCATGTTTACAAGTCGGACAGGAGCAGAAACACACCTACCTTCCCCTGGAGG
TCTGTAACATTGTGGCAGGACAAAGATGTATTAAAAAATTAACGGACAATCAGACCT
CAACCATGATCAGAGCGACTGCTAGGTCGGCGCCCGATCGGCAAGAAGAGATTAGC
AAATTGATGCGAAGTGCAAGTTTCAACACAGATCCATACGTCCGTGAATTTGGAATC
ATGGTCAAAGATGAGATGACAGACGTGACTGGGCGGGTGCTGCAGCCGCCCTCCATC
CTCTACGGGGGCAGGAATAAAGCTATTGCGACCCCTGTCCAGGGCGTCTGGGACATG
CGGAACAAGCAGTTCCACACGGGCATCGAGATCAAGGTGTGGGCCATTGCGTGCTTC
GCCCCCCAGCGCCAGTGCACGGAAGTCCATCTGAAGTCCTTCACAGAGCAGCTCAGA
AAGATCTCGAGAGACGCCGGCATGCCCATCCAGGGCCAGCCGTGCTTCTGCAAATAC
GCGCAGGGGGCGGACAGCGTGGAGCCCATGTTCCGGCACCTGAAGAACACGTATGC
GGGCCTGCAGCTGGTGGTGGTCATCCTGCCCGGCAAGACGCCCGTGTACGCCGAGGT
CAAGCGCGTGGGAGACACGGTGCTGGGGATGGCCACGCAGTGCGTGCAGATGAAGA
ACGTGCAGAGGACCACGCCACAGACCCTGTCCAACCTCTGCCTGAAGATCAACGTCA
AGCTGGGAGGCGTGAACAACATCCTGCTGCCCCAGGGCAGGCCGCCGGTGTTCCAGC
AGCCCGTCATCTTTCTGGGAGCAGCCGTCACTCACCCCCCCGCCGGGGATGGGAAGA
AGCCCTCCATTGCCGCCGTGGTGGGCAGCATGGACGCCCACCCCAATCGCTACTGCG
CCACCGTGCGCGTGCAGCAGCACCGGCAGGAGATCATACAAGACCTGGCCGCCATG
GTCCGCGAGCTCCTCATCCAGTTCTACAAGTCCACGCGCTTCAAGCCCACCCGCATC
ATCTTCTACCGCGCCGGTGTCTCTGAAGGCCAGTTCCAGCAGGTTCTCCACCACGAGT
TGCTGGCCATCCGTGAGGCCTGTATCAAGCTAGAAAAAGACTACCAGCCCGGGATCA
CCTTCATCGTGGTGCAGAAGAGGCACCACACCCGGCTCTTCTGCACTGACAAGAACG
AGCGGGTTGGGAAAAGTGGAAACATTCCAGCAGGCACGACTGTGGACACGAAAATC
ACCCACCCCACCGAGTTCGACTTCTACCTGTGTAGTCACGCTGGCATCCAGGGGACA
AGCAGGCCTTCGCACTATCACGTCCTCTGGGACGACAATCGTTTCTCCTCTGATGAGC
TGCAGATCCTAACCTACCAGCTGTGTCACACCTACGTGCGCTGCACACGCTCCGTGTC
CATCCCAGCGCCAGCATACTACGCTCACCTGGTGGCCTTCCGGGCCAGGTACCACCT
GGTGGATAAGGAACATGACAGTGCTGAAGGAAGCCATACCTCTGGGCAGAGTAACG
GGCGAGACCACCAAGCACTGGCCAAGGCGGTCCAGGTTCACCAAGACACTCTGCGC
ACCATGTACTTTGCTTGA
ADAR-4GS1-hAgo2-D597A/D669A-ΔN51
(SEQ ID NO: 108)
ATGGACTACAAAGACGATGACGACAAGGCGGCCGCAcagctgcatttaccgcaggttttagctgacgctg
tctcacgcctggtcctgggtaagtttggtgacctgaccgacaacttctcctcccctcacgctcgcagaaaagtgctggctggagtcgtcatga
caacaggcacagatgttaaagatgccaaggtgataagtgtttctacaggaGGCaaatgtattaatggtgaatacatgagtgatcgtggcctt
gcattaaatgactgccatgcagaaataatatctcggagatccttgctcagatttctttatacacaacttgagctttacttaaataacaaagatga
tcaaaaaagatccatctttcagaaatcagagcgaggggggtttaggctgaaggagaatgtccagtttcatctgtacatcagcacctctccctgtg
gagatgccagaatcttctcaccacatgagccaatcctggaagaaccagcagatagacacccaaatcgtaaagcaagaggacagctacgga
ccaaaatagagtctggtCaggggacgattccagtgcgctccaatgcgagcatccaaacgtgggacggggtgctgcaaggggagcggct
gctcaccatgtcctgcagtgacaagattgcacgctggaacgtggtgggcatccagggatcActgctcagcattttcgtggagcccatttactt
ctcgagcatcatcctgggcagcctttaccacggggaccacctttccagggccatgtaccagcggatctccaacatagaggacctgccacctc
tctacaccctcaacaagcctttgctcagtggcatcagcaatgcagaagcacggcagccagggaaggcccccaacttcagtgtcaactggac
ggtaggcgactccgctattgaggtcatcaacgccacgactgggaaggatgagctgggccgcgcgtcccgcctgtgtaagcacgcgttgta
ctgtcgctggatgcgtgtgcacggcaaggttccctcccacttactacgctccaagattaccaagcccaacgtgtaccatgagtccaagctggc
ggcaaaggagtaccaggccgccaaggcgcgtctgttcacagccttcatcaaggcggggctgggggcctgggtggagaagcccaccgag
caggaccagttctcactcacg
GGAGGTGGCGGTAGTCTGCAGCTGCCTCCACTTGAAAGACTGACACTGGGATCC
ATTGACATCTATCATTATGAATTGGATATCAAGCCAGAGAAGTGCCCGAGGAGAGTT
AACAGGGAAATCGTGGAACACATGGTCCAGCACTTTAAAACACAGATCTTTGGGGAT
CGGAAGCCCGTGTTTGACGGCAGGAAGAATCTATACACAGCCATGCCCCTTCCGATT
GGGAGGGACAAGGTGGAGCTGGAGGTCACGCTGCCAGGAGAAGGCAAGGATCGCAT
CTTCAAGGTGTCCATCAAGTGGGTGTCCTGCGTGAGCTTGCAGGCGTTACACGATGC
ACTTTCAGGGCGGCTGCCCAGCGTCCCTTTTGAGACGATCCAGGCCCTGGACGTGGT
CATGAGGCACTTGCCATCCATGAGGTACACCCCCGTGGGCCGCTCCTTCTTCACCGC
GTCCGAAGGCTGCTCTAACCCTCTTGGCGGGGGCCGAGAAGTGTGGTTTGGCTTCCA
TCAGTCCGTCCGGCCTTCTCTCTGGAAAATGATGCTGAATATTGATGTGTCAGCAACA
GCGTTTTACAAGGCACAGCCAGTAATCGAGTTTGTTTGTGAAGTTTTGGATTTTAAAA
GTATTGAAGAACAACAAAAACCTCTGACAGATTCCCAAAGGGTAAAGTTTACCAAA
GAAATTAAAGGTCTAAAGGTGGAGATAACGCACTGTGGGCAGATGAAGAGGAAGTA
CCGCGTCTGCAATGTGACCCGGCGGCCCGCCAGTCACCAAACATTCCCGCTGCAGCA
GGAGAGCGGGCAGACGGTGGAGTGCACGGTGGCCCAGTATTTCAAGGACAGGCACA
AGTTGGTTCTGCGCTACCCCCACCTCCCATGTTTACAAGTCGGACAGGAGCAGAAAC
ACACCTACCTTCCCCTGGAGGTCTGTAACATTGTGGCAGGACAAAGATGTATTAAAA
AATTAACGGACAATCAGACCTCAACCATGATCAGAGCGACTGCTAGGTCGGCGCCCG
ATCGGCAAGAAGAGATTAGCAAATTGATGCGAAGTGCAAGTTTCAACACAGATCCA
TACGTCCGTGAATTTGGAATCATGGTCAAAGATGAGATGACAGACGTGACTGGGCGG
GTGCTGCAGCCGCCCTCCATCCTCTACGGGGGCAGGAATAAAGCTATTGCGACCCCT
GTCCAGGGCGTCTGGGACATGCGGAACAAGCAGTTCCACACGGGCATCGAGATCAA
GGTGTGGGCCATTGCGTGCTTCGCCCCCCAGCGCCAGTGCACGGAAGTCCATCTGAA
GTCCTTCACAGAGCAGCTCAGAAAGATCTCGAGAGACGCCGGCATGCCCATCCAGG
GCCAGCCGTGCTTCTGCAAATACGCGCAGGGGGCGGACAGCGTGGAGCCCATGTTCC
GGCACCTGAAGAACACGTATGCGGGCCTGCAGCTGGTGGTGGTCATCCTGCCCGGCA
AGACGCCCGTGTACGCCGAGGTCAAGCGCGTGGGAGACACGGTGCTGGGGATGGCC
ACGCAGTGCGTGCAGATGAAGAACGTGCAGAGGACCACGCCACAGACCCTGTCCAA
CCTCTGCCTGAAGATCAACGTCAAGCTGGGAGGCGTGAACAACATCCTGCTGCCCCA
GGGCAGGCCGCCGGTGTTCCAGCAGCCCGTCATCTTTCTGGGAGCAGCCGTCACTCA
CCCCCCCGCCGGGGATGGGAAGAAGCCCTCCATTGCCGCCGTGGTGGGCAGCATGG
ACGCCCACCCCAATCGCTACTGCGCCACCGTGCGCGTGCAGCAGCACCGGCAGGAG
ATCATACAAGACCTGGCCGCCATGGTCCGCGAGCTCCTCATCCAGTTCTACAAGTCC
ACGCGCTTCAAGCCCACCCGCATCATCTTCTACCGCGCCGGTGTCTCTGAAGGCCAG
TTCCAGCAGGTTCTCCACCACGAGTTGCTGGCCATCCGTGAGGCCTGTATCAAGCTA
GAAAAAGACTACCAGCCCGGGATCACCTTCATCGTGGTGCAGAAGAGGCACCACAC
CCGGCTCTTCTGCACTGACAAGAACGAGCGGGTTGGGAAAAGTGGAAACATTCCAG
CAGGCACGACTGTGGACACGAAAATCACCCACCCCACCGAGTTCGACTTCTACCTGT
GTAGTCACGCTGGCATCCAGGGGACAAGCAGGCCTTCGCACTATCACGTCCTCTGGG
ACGACAATCGTTTCTCCTCTGATGAGCTGCAGATCCTAACCTACCAGCTGTGTCACAC
CTACGTGCGCTGCACACGCTCCGTGTCCATCCCAGCGCCAGCATACTACGCTCACCT
GGTGGCCTTCCGGGCCAGGTACCACCTGGTGGATAAGGAACATGACAGTGCTGAAG
GAAGCCATACCTCTGGGCAGAGTAACGGGCGAGACCACCAAGCACTGGCCAAGGCG
GTCCAGGTTCACCAAGACACTCTGCGCACCATGTACTTTGCTTGA
FLAG-ADAR-GGGGS-NES-hAgo3
(SEQ ID NO: 109)
ATG GACTACAAAGACGATGACGACAAG GCGGCCGCA
Cagctgcatttaccgcaggttttagctgacgctgtctcacgcctggtcctgggtaagtttggtgacctgaccgacaacttctcctcccctcacg
ctcgcagaaaagtgctggctggagtcgtcatgacaacaggcacagatgttaaagatgccaaggtgataagtgtttctacaggaacaaaatgt
attaatggtgaatacatgagtgatcgtggccttgcattaaatgactgccatgcagaaataatatctcggagatccttgctcagatttctttatac
acaacttgagctttacttaaataacaaagatgatcaaaaaagatccatctttcagaaatcagagcgaggggggtttaggctgaaggagaatgtcc
agtttcatctgtacatcagcacctctccctgtggagatgccagaatcttctcaccacatgagccaatcctggaagaaccagcagatagacacc
caaatcgtaaagcaagaggacagctacggaccaaaatagagtctggtCaggggacgattccagtgcgctccaatgcgagcatccaaacgt
gggacggggtgctgcaaggggagcggctgctcaccatgtcctgcagtgacaagattgcacgctggaacgtggtgggcatccagggatc
Actgctcagcattttcgtggagcccatttacttctcgagcatcatcctgggcagcctttaccacggggaccacctttccagggccatgtaccag
cggatctccaacatagaggacctgccacctctctacaccctcaacaagcctttgctcagtggcatcagcaatgcagaagcacggcagccag
ggaaggcccccaacttcagtgtcaactggacggtaggcgactccgctattgaggtcatcaacgccacgactgggaaggatgagctgggcc
gcgcgtcccgcctgtgtaagcacgcgttgtactgtcgctggatgcgtgtgcacggcaaggttccctcccacttactacgctccaagattacca
agcccaacgtgtaccatgagtccaagctggcggcaaaggagtaccaggccgccaaggcgcgtctgttcacagccttcatcaaggcgggg
ctgggggcctgggtggagaagcccaccgagcaggaccagttctcactcacg
GGAGGTGGCGGTAGTCTGCAGCTGCCTCCACTTGAAAGACTGACACTGGGATCC
gaaatcggctccgcaggacccgctggggcccagcccctactcatggtgcccagaagacctggctatggcaccatgggcaaacccattaaa
ctgctggctaactgttttcaagttgaaatcccaaagattgatgtctacctctatgaggtagatattaaaccagacaagtgtcctaggagagtgaa
cagggaggtggttgactcaatggttcagcattttaaagtaactatatttggagaccgtagaccagtttatgatggaaaaagaagtctttacaccg
ccaatccacttcctgtggcaactacaggggtagatttagacgttactttacctggggaaggtggaaaagatcgacctttcaaggtgtcaatcaa
atttgtctctcgggtgagttggcacctactgcatgaagtactgacaggacggaccttgcctgagccactggaattagacaagccaatcagcac
taaccctgtccatgccgttgatgtggtgctacgacatctgccctccatgaaatacacacctgtggggcgttcatttttctccgctccagaaggat
atgaccaccctctgggagggggcagggaagtgtggtttggattccatcagtctgttcggcctgccatgtggaaaatgatgcttaatatcgatgt
ttctgccactgccttctacaaagcacaacctgtaattcagttcatgtgtgaagttcttgatattcataatattgatgagcaaccaagacctctga
ctgattctcatcgggtaaaattcaccaaagagataaaaggtttgaaggttgaagtgactcattgtggaacaatgagacggaaataccgtgtttgt
aatgtaacaaggaggcctgccagtcatcaaacctttcctttacagttagaaaacggccaaactgtggagagaacagtagcgcagtatttcagag
aaaagtatactcttcagctgaagtacccgcaccttccctgtctgcaagtcgggcaggaacagaaacacacctacctgccactagaagtctgta
atattgtggcagggcaacgatgtatcaagaagctaacagacaatcagacttccactatgatcaaggcaacagcaagatctgcaccagataga
caagaggaaattagcagattggtaagaagtgcaaattatgaaacagatccatttgttcaggagtttcaatttaaagttcgggatgaaatggctca
tgtaactggacgcgtacttccagcacctatgctccagtatggaggacggaatcggacagtagcaacaccgagccatggagtatgggacatg
cgagggaaacaattccacacaggagttgaaatcaaaatgtgggctatcgcttgttttgccacacagaggcagtgcagagaagaaatattgaa
gggtttcacagaccagctgcgtaagatttctaaggatgcagggatgcccatccagggccagccatgcttctgcaaatatgcacagggggca
gacagcgtagagcccatgttccggcatctcaagaacacatattctggcctacagcttattatcgtcatcctgccggggaagacaccagtgtat
gcggaagtgaaacgtgtaggagacacacttttgggtatggctacacaatgtgttcaagtcaagaatgtaataaaaacatctcctcaaactctgt
caaacttgtgcctaaagataaatgttaaactcggagggatcaataatattcttgtacctcatcaaagaccttctgtgttccagcaaccagtgatc
tttttgggagccgatgtcactcatccacctgctggtgatggaaagaagccttctattgctgctgttgtaggtagtatggatgcacacccaagcag
atactgtgccacagtaagagttcagagaccccgacaggagatcatccaggacttggcctccatggtccgggaacttcttattcaattttataagt
caactcggttcaagcctactcgtatcatcttttatcgggatggtgtttcagaggggcagtttaggcaggtattatattatgaactactagcaatt
cgagaagcctgcatcagtttggagaaagactatcaacctggaataacctacattgtagttcagaagagacatcacactcgattattttgtgctga
taggacagaaagggttggaagaagtggcaatatcccagctggaacaacagttgatacagacattacacacccatatgagttcgatttttacctct
gtagccatgctggaatacagggtaccagtcgtccttcacactatcatgttttatgggatgataactgctttactgcagatgaacttcagctgcta
acttaccagctctgccacacttacgtacgctgtacacgatctgtttctatacctgcaccagcgtattatgctcacctggtagcatttagagccag
atatcatcttgtggacaaagaacatgacagtgctgaaggaagtcacgtttcaggacaaagcaatgggcgagatccacaagctcttgccaaggctg
tacagattcaccaagataccttacgcacaatgtacttcgcttaa
FLAG-ADAR-GGGGS-NES-hAgo4
(SEQ ID NO: 110)
ATG GACTACAAAGACGATGACGACAAG GCGGCCGCA
Cagctgcatttaccgcaggttttagctgacgctgtctcacgcctggtcctgggtaagtttggtgacctgaccgacaacttctcctcccctcacg
ctcgcagaaaagtgctggctggagtcgtcatgacaacaggcacagatgttaaagatgccaaggtgataagtgtttctacaggaacaaaatgt
attaatggtgaatacatgagtgatcgtggccttgcattaaatgactgccatgcagaaataatatctcggagatccttgctcagatttctttatac
acaacttgagctttacttaaataacaaagatgatcaaaaaagatccatctttcagaaatcagagcgaggggggtttaggctgaaggagaatgtcc
agtttcatctgtacatcagcacctctccctgtggagatgccagaatcttctcaccacatgagccaatcctggaagaaccagcagatagacacc
caaatcgtaaagcaagaggacagctacggaccaaaatagagtctggtCaggggacgattccagtgcgctccaatgcgagcatccaaacgt
gggacggggtgctgcaaggggagcggctgctcaccatgtcctgcagtgacaagattgcacgctggaacgtggtgggcatccagggatc
Actgctcagcattttcgtggagcccatttacttctcgagcatcatcctgggcagcctttaccacggggaccacctttccagggccatgtaccag
cggatctccaacatagaggacctgccacctctctacaccctcaacaagcctttgctcagtggcatcagcaatgcagaagcacggcagccag
ggaaggcccccaacttcagtgtcaactggacggtaggcgactccgctattgaggtcatcaacgccacgactgggaaggatgagctgggcc
gcgcgtcccgcctgtgtaagcacgcgttgtactgtcgctggatgcgtgtgcacggcaaggttccctcccacttactacgctccaagattacca
agcccaacgtgtaccatgagtccaagctggcggcaaaggagtaccaggccgccaaggcgcgtctgttcacagccttcatcaaggcgggg
ctgggggcctgggtggagaagcccaccgagcaggaccagttctcactcacg
GGAGGTGGCGGTAGTCTGCAGCTGCCTCCACTTGAAAGACTGACACTGGGATCC
gaggcgctgggacccggacctccggctagcctgtttcagccacctcgtcgtcctggccttggaactgttggaaaaccaattcgactgttagc
caatcattttcaggttcagattcctaaaatagatgtgtatcactatgatgtggatattaagcctgaaaaacggcctcgtagagtcaacagggagg
tagtagatacaatggtgcggcacttcaagatgcaaatatttggtgatcggcagcctgggtatgatggcaaaagaaacatgtacacagcacatc
cactaccaattggacgggatagggttgatatggaggtgactcttccaggcgagggtaaagaccaaacatttaaagtgtctgttcagtgggtgt
cagttgtgagccttcagttgcttttagaagctttggctgggcacttgaatgaagtcccagatgactcagtacaagcacttgatgttatcacaaga
caccttccctccatgaggtacaccccagtgggccgttcctttttctcacccccggaaggttactaccaccctctgggagggggcagggaggt
ctggtttggttttcatcagtctgtgagacctgccatgtggaatatgatgctcaacattgatgtatctgcaactgctttctaccgggctcagccta
tcattgagttcatgtgtgaggttttagacattcagaacatcaatgaacagaccaaacctctaacagactcccagcgtgtcaaatttaccaaagaa
atcagaggtctcaaagttgaggtgacccactgtggacagatgaaacgaaaataccgagtttgtaatgtgactagacggccagccagtcatcaaa
cttttcctttgcagctagaaaacggtcaagctatggaatgtacagtagctcaatattttaagcaaaagtatagtctgcaactgaaatacccccat
cttccctgtctccaagtgggacaagaacaaaagcatacatacttgccactcgaggtctgtaatatagtggcaggacagcgatgtatcaagaagc
tcacagacaatcagacttccacaatgatcaaagctacagcaagatctgctcctgacagacaggaagagatcagtagactggtgaagagcaa
cagtatggtgggtggacctgatccataccttaaagaatttggtattgttgtccacaatgaaatgacagagctcacaggcagggtacttccagca
ccaatgctgcaatatggaggccggaataaaacagtagccacacccaaccagggtgtctgggacatgcgaggaaagcagttttatgctggca
ttgaaattaaagtttgggcagttgcttgttttgcacctcagaaacaatgtagggaagatttactaaagagtttcactgaccagctgcgtaaaatc
tctaaggatgcaggaatgcccatccagggtcagccatgtttctgcaagtatgcacaaggtgcagacagtgtggagcctatgtttaaacatctga
aaatgacttatgtgggcctacagctaatagtggttatcctgcctggaaagacaccagtatatgcggaggtgaaacgtgttggagatacccttct
aggtatggccacacagtgtgtccaggtaaaaaatgtagtgaagacctcacctcaaaccctttccaatctttgcctgaagataaatgcaaaactt
ggaggaattaacaatgtgcttgtgcctcatcaaaggccctcggtgttccagcagcctgtcatcttcctgggagcggatgtcacacacccccca
gcaggggatgggaagaaaccttccattgctgctgtggttggcagtatggatggccaccccagccggtactgtgccaccgttcgggtgcaga
cttcccggcaggagatctcccaagagctcctctacagtcaagaggtcatccaggacctgactaacatggttcgagagctgctgattcagttct
acaaatccacacgcttcaaacccactcggatcatctattaccgtggaggggtatctgagggacaaatgaaacaggtagcttggccagaacta
atagcaattcgaaaggcatgtattagcttggaagaagattaccggccaggaataacttatattgtggtgcaaaaaagacatcacacacgactct
tctgtgcagataaaacagaaagggtagggaaaagtggcaatgtaccagcaggcactacagtggatagtaccatcacacatccatctgagttt
gacttttacctctgtagtcatgcaggaattcagggaaccagccgtccctcacattaccaggtcttgtgggatgacaactgcttcactgcagatg
aactccagctactgacttaccagctgtgtcacacctatgtgaggtgcactcgctcagtctctattccagcccctgcatattatgcccggcttgta
gcatttagggcaaggtatcatctggtggataaagatcatgacagtgcggaaggcagtcatgtgtcaggacagagcaacggccgggatcctca
ggccttggctaaggctgtgcaaatccaccatgatacccagcacacgatgtattttgcctga
APOBEC3A-Y132D-4GS1-NES-Ago2-D597A/D669A
(SEQ ID NO: 111)
ATGGACTACAAAGACGATGACGACAAGGCGGCCGCAgaagccagcccagcatccgggcccagacact
tgatggatccacacatattcacttccaactttaacaatggcattggaaggcataagacctacctgtgctacgaagtggagcgcctggacaatg
gcacctcggtcaagatggaccagcacaggggctttctacacaaccaggctaagaatcttctctgtggcttttacggccgccatgcggagctg
cgcttcttggacctggttccttctttgcagttggacccggcccagatctacagggtcacttggttcatctcctggagcccctgcttctcctgggg
ctgtgccggggaagtgcgtgcgttccttcaggagaacacacacgtgagactgcgtatcttcgctgcccgcatctatgatGacgaccccctata
taaggaggcactgcaaatgctgcgggatgctggggcccaagtctccatcatgacctacgatgaatttaagcactgctgggacacctttgtgg
accaccagggatgtcccttccagccctgggatggactagatgagcacagccaagccctgagtgggaggctgcgggccattctccagaatc
agggaaacGGAGGTGGCGGTAGTCTGCAGCTGCCTCCACTTGAAAGACTGACACTGGGA
TCCTACTCGGGAGCCGGCCCCGCACTTGCACCTCCTGCGCCGCCGCCCCCCATCCAA
GGATATGCCTTCAAGCCTCCACCTAGACCCGACTTTGGGACCTCCGGGAGAACAATC
AAATTACAGGCCAATTTCTTCGAAATGGACATCCCCAAAATTGACATCTATCATTAT
GAATTGGATATCAAGCCAGAGAAGTGCCCGAGGAGAGTTAACAGGGAAATCGTGGA
ACACATGGTCCAGCACTTTAAAACACAGATCTTTGGGGATCGGAAGCCCGTGTTTGA
CGGCAGGAAGAATCTATACACAGCCATGCCCCTTCCGATTGGGAGGGACAAGGTGG
AGCTGGAGGTCACGCTGCCAGGAGAAGGCAAGGATCGCATCTTCAAGGTGTCCATC
AAGTGGGTGTCCTGCGTGAGCTTGCAGGCGTTACACGATGCACTTTCAGGGCGGCTG
CCCAGCGTCCCTTTTGAGACGATCCAGGCCCTGGACGTGGTCATGAGGCACTTGCCA
TCCATGAGGTACACCCCCGTGGGCCGCTCCTTCTTCACCGCGTCCGAAGGCTGCTCTA
ACCCTCTTGGCGGGGGCCGAGAAGTGTGGTTTGGCTTCCATCAGTCCGTCCGGCCTTC
TCTCTGGAAAATGATGCTGAATATTGATGTGTCAGCAACAGCGTTTTACAAGGCACA
GCCAGTAATCGAGTTTGTTTGTGAAGTTTTGGATTTTAAAAGTATTGAAGAACAACA
AAAACCTCTGACAGATTCCCAAAGGGTAAAGTTTACCAAAGAAATTAAAGGTCTAA
AGGTGGAGATAACGCACTGTGGGCAGATGAAGAGGAAGTACCGCGTCTGCAATGTG
ACCCGGCGGCCCGCCAGTCACCAAACATTCCCGCTGCAGCAGGAGAGCGGGCAGAC
GGTGGAGTGCACGGTGGCCCAGTATTTCAAGGACAGGCACAAGTTGGTTCTGCGCTA
CCCCCACCTCCCATGTTTACAAGTCGGACAGGAGCAGAAACACACCTACCTTCCCCT
GGAGGTCTGTAACATTGTGGCAGGACAAAGATGTATTAAAAAATTAACGGACAATC
AGACCTCAACCATGATCAGAGCGACTGCTAGGTCGGCGCCCGATCGGCAAGAAGAG
ATTAGCAAATTGATGCGAAGTGCAAGTTTCAACACAGATCCATACGTCCGTGAATTT
GGAATCATGGTCAAAGATGAGATGACAGACGTGACTGGGCGGGTGCTGCAGCCGCC
CTCCATCCTCTACGGGGGCAGGAATAAAGCTATTGCGACCCCTGTCCAGGGCGTCTG
GGACATGCGGAACAAGCAGTTCCACACGGGCATCGAGATCAAGGTGTGGGCCATTG
CGTGCTTCGCCCCCCAGCGCCAGTGCACGGAAGTCCATCTGAAGTCCTTCACAGAGC
AGCTCAGAAAGATCTCGAGAGACGCCGGCATGCCCATCCAGGGCCAGCCGTGCTTCT
GCAAATACGCGCAGGGGGCGGACAGCGTGGAGCCCATGTTCCGGCACCTGAAGAAC
ACGTATGCGGGCCTGCAGCTGGTGGTGGTCATCCTGCCCGGCAAGACGCCCGTGTAC
GCCGAGGTCAAGCGCGTGGGAGACACGGTGCTGGGGATGGCCACGCAGTGCGTGCA
GATGAAGAACGTGCAGAGGACCACGCCACAGACCCTGTCCAACCTCTGCCTGAAGA
TCAACGTCAAGCTGGGAGGCGTGAACAACATCCTGCTGCCCCAGGGCAGGCCGCCG
GTGTTCCAGCAGCCCGTCATCTTTCTGGGAGCAGCCGTCACTCACCCCCCCGCCGGG
GATGGGAAGAAGCCCTCCATTGCCGCCGTGGTGGGCAGCATGGACGCCCACCCCAAT
CGCTACTGCGCCACCGTGCGCGTGCAGCAGCACCGGCAGGAGATCATACAAGACCT
GGCCGCCATGGTCCGCGAGCTCCTCATCCAGTTCTACAAGTCCACGCGCTTCAAGCC
CACCCGCATCATCTTCTACCGCGCCGGTGTCTCTGAAGGCCAGTTCCAGCAGGTTCTC
CACCACGAGTTGCTGGCCATCCGTGAGGCCTGTATCAAGCTAGAAAAAGACTACCAG
CCCGGGATCACCTTCATCGTGGTGCAGAAGAGGCACCACACCCGGCTCTTCTGCACT
GACAAGAACGAGCGGGTTGGGAAAAGTGGAAACATTCCAGCAGGCACGACTGTGGA
CACGAAAATCACCCACCCCACCGAGTTCGACTTCTACCTGTGTAGTCACGCTGGCAT
CCAGGGGACAAGCAGGCCTTCGCACTATCACGTCCTCTGGGACGACAATCGTTTCTC
CTCTGATGAGCTGCAGATCCTAACCTACCAGCTGTGTCACACCTACGTGCGCTGCAC
ACGCTCCGTGTCCATCCCAGCGCCAGCATACTACGCTCACCTGGTGGCCTTCCGGGC
CAGGTACCACCTGGTGGATAAGGAACATGACAGTGCTGAAGGAAGCCATACCTCTG
GGCAGAGTAACGGGCGAGACCACCAAGCACTGGCCAAGGCGGTCCAGGTTCACCAA
GACACTCTGCGCACCATGTACTTTGCTTGA
APOBEC3A-Y132R-4GS1-NES-Ago2-D597A_D669A
(SEQ ID NO: 112)
ATGGACTACAAAGACGATGACGACAAGGCGGCCGCAgaagccagcccagcatccgggcccagacact
tgatggatccacacatattcacttccaactttaacaatggcattggaaggcataagacctacctgtgctacgaagtggagcgcctggacaatg
gcacctcggtcaagatggaccagcacaggggctttctacacaaccaggctaagaatcttctctgtggcttttacggccgccatgcggagctg
cgcttcttggacctggttccttctttgcagttggacccggcccagatctacagggtcacttggttcatctcctggagcccctgcttctcctgggg
ctgtgccggggaagtgcgtgcgttccttcaggagaacacacacgtgagactgcgtatcttcgctgcccgcatctatgatCGcgaccccctata
taaggaggcactgcaaatgctgcgggatgctggggcccaagtctccatcatgacctacgatgaatttaagcactgctgggacacctttgtgg
accaccagggatgtcccttccagccctgggatggactagatgagcacagccaagccctgagtgggaggctgcgggccattctccagaatc
agggaaacGGAGGTGGCGGTAGTCTGCAGCTGCCTCCACTTGAAAGACTGACACTGGGA
TCCTACTCGGGAGCCGGCCCCGCACTTGCACCTCCTGCGCCGCCGCCCCCCATCCAA
GGATATGCCTTCAAGCCTCCACCTAGACCCGACTTTGGGACCTCCGGGAGAACAATC
AAATTACAGGCCAATTTCTTCGAAATGGACATCCCCAAAATTGACATCTATCATTAT
GAATTGGATATCAAGCCAGAGAAGTGCCCGAGGAGAGTTAACAGGGAAATCGTGGA
ACACATGGTCCAGCACTTTAAAACACAGATCTTTGGGGATCGGAAGCCCGTGTTTGA
CGGCAGGAAGAATCTATACACAGCCATGCCCCTTCCGATTGGGAGGGACAAGGTGG
AGCTGGAGGTCACGCTGCCAGGAGAAGGCAAGGATCGCATCTTCAAGGTGTCCATC
AAGTGGGTGTCCTGCGTGAGCTTGCAGGCGTTACACGATGCACTTTCAGGGCGGCTG
CCCAGCGTCCCTTTTGAGACGATCCAGGCCCTGGACGTGGTCATGAGGCACTTGCCA
TCCATGAGGTACACCCCCGTGGGCCGCTCCTTCTTCACCGCGTCCGAAGGCTGCTCTA
ACCCTCTTGGCGGGGGCCGAGAAGTGTGGTTTGGCTTCCATCAGTCCGTCCGGCCTTC
TCTCTGGAAAATGATGCTGAATATTGATGTGTCAGCAACAGCGTTTTACAAGGCACA
GCCAGTAATCGAGTTTGTTTGTGAAGTTTTGGATTTTAAAAGTATTGAAGAACAACA
AAAACCTCTGACAGATTCCCAAAGGGTAAAGTTTACCAAAGAAATTAAAGGTCTAA
AGGTGGAGATAACGCACTGTGGGCAGATGAAGAGGAAGTACCGCGTCTGCAATGTG
ACCCGGCGGCCCGCCAGTCACCAAACATTCCCGCTGCAGCAGGAGAGCGGGCAGAC
GGTGGAGTGCACGGTGGCCCAGTATTTCAAGGACAGGCACAAGTTGGTTCTGCGCTA
CCCCCACCTCCCATGTTTACAAGTCGGACAGGAGCAGAAACACACCTACCTTCCCCT
GGAGGTCTGTAACATTGTGGCAGGACAAAGATGTATTAAAAAATTAACGGACAATC
AGACCTCAACCATGATCAGAGCGACTGCTAGGTCGGCGCCCGATCGGCAAGAAGAG
ATTAGCAAATTGATGCGAAGTGCAAGTTTCAACACAGATCCATACGTCCGTGAATTT
GGAATCATGGTCAAAGATGAGATGACAGACGTGACTGGGCGGGTGCTGCAGCCGCC
CTCCATCCTCTACGGGGGCAGGAATAAAGCTATTGCGACCCCTGTCCAGGGCGTCTG
GGACATGCGGAACAAGCAGTTCCACACGGGCATCGAGATCAAGGTGTGGGCCATTG
CGTGCTTCGCCCCCCAGCGCCAGTGCACGGAAGTCCATCTGAAGTCCTTCACAGAGC
AGCTCAGAAAGATCTCGAGAGACGCCGGCATGCCCATCCAGGGCCAGCCGTGCTTCT
GCAAATACGCGCAGGGGGCGGACAGCGTGGAGCCCATGTTCCGGCACCTGAAGAAC
ACGTATGCGGGCCTGCAGCTGGTGGTGGTCATCCTGCCCGGCAAGACGCCCGTGTAC
GCCGAGGTCAAGCGCGTGGGAGACACGGTGCTGGGGATGGCCACGCAGTGCGTGCA
GATGAAGAACGTGCAGAGGACCACGCCACAGACCCTGTCCAACCTCTGCCTGAAGA
TCAACGTCAAGCTGGGAGGCGTGAACAACATCCTGCTGCCCCAGGGCAGGCCGCCG
GTGTTCCAGCAGCCCGTCATCTTTCTGGGAGCAGCCGTCACTCACCCCCCCGCCGGG
GATGGGAAGAAGCCCTCCATTGCCGCCGTGGTGGGCAGCATGGACGCCCACCCCAAT
CGCTACTGCGCCACCGTGCGCGTGCAGCAGCACCGGCAGGAGATCATACAAGACCT
GGCCGCCATGGTCCGCGAGCTCCTCATCCAGTTCTACAAGTCCACGCGCTTCAAGCC
CACCCGCATCATCTTCTACCGCGCCGGTGTCTCTGAAGGCCAGTTCCAGCAGGTTCTC
CACCACGAGTTGCTGGCCATCCGTGAGGCCTGTATCAAGCTAGAAAAAGACTACCAG
CCCGGGATCACCTTCATCGTGGTGCAGAAGAGGCACCACACCCGGCTCTTCTGCACT
GACAAGAACGAGCGGGTTGGGAAAAGTGGAAACATTCCAGCAGGCACGACTGTGGA
CACGAAAATCACCCACCCCACCGAGTTCGACTTCTACCTGTGTAGTCACGCTGGCAT
CCAGGGGACAAGCAGGCCTTCGCACTATCACGTCCTCTGGGACGACAATCGTTTCTC
CTCTGATGAGCTGCAGATCCTAACCTACCAGCTGTGTCACACCTACGTGCGCTGCAC
ACGCTCCGTGTCCATCCCAGCGCCAGCATACTACGCTCACCTGGTGGCCTTCCGGGC
CAGGTACCACCTGGTGGATAAGGAACATGACAGTGCTGAAGGAAGCCATACCTCTG
GGCAGAGTAACGGGCGAGACCACCAAGCACTGGCCAAGGCGGTCCAGGTTCACCAA
GACACTCTGCGCACCATGTACTTTGCTTGA

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

1. A system for modifying a target RNA, the system comprising:
a. a polypeptide or a nucleic acid encoding a polypeptide, wherein the polypeptide comprises:
i. a first domain comprising a catalytic domain of an RNA modifying enzyme, wherein the RNA modifying enzyme is not a nuclease;
ii. a second domain comprising a MID domain of an Argonaute (Ago) protein; and
b. an oligonucleotide, optionally the oligonucleotide is double-stranded and comprises a double-stranded region of at least 17 base-pairs.

2. The system of claim 1, wherein the second domain further comprises a PAZ domain of an Ago.

3. The system of claim 1, wherein the second domain further comprises a PIWI domain of an Ago, optionally the PIWI domain lacks nuclease activity.

4. (canceled)

5. (canceled)

6. (canceled)

7. (canceled)

8. The system of claim 1, wherein the second domain comprises an amino acid sequence having a mutation at one or more positions selected from the group consisting of D597 and D699 of human Ago2 amino acid sequence, or a corresponding position in a homologous or orthologous Ago protein, optionally, the second domain comprises an amino acid sequence having a mutation at one or more positions selected from the group consisting of D597A and D699A of human Ago2 amino acid sequence, or a corresponding position in a homologous or orthologous Ago protein.

9. (canceled)

10. The system of claim 1, wherein the RNA modifying enzyme is an RNA deaminase, an RNA methylase, or an RNA demethylase.

11. The system of claim 10, wherein the catalytic domain of the RNA modifying enzyme is a deaminase domain of an RNA deaminase.

12. The system of claim 11, wherein the RNA deaminase is Adenosine Deaminase Acting on RNA (ADAR) or a cytidine deaminase, optionally the cytidine deaminase is an apolipoprotein B mRNA editing enzyme catalytic polypeptide-like (APOBEC).

13. (canceled)

14. (canceled)

15. (canceled)

16. The system of claim 1, wherein: (i) the first domain comprises an amino acid sequence having a mutation at one or more positions selected from the group consisting of T375, E448 and E488 of human ADAR2 (hADAR2) amino acid sequence, or a corresponding position in a homologous or orthologous ADAR protein: (ii) the first domain comprises an amino acid sequence having a mutation at one or more positions selected from the group consisting of T339, R348, A353, V351, V355, T375, K376, E396, S397, E438, F442, H443, L444, Y445, T448, C451, R455, S486, Q488, R510, I520, V525, P539, G593, K594 and E1008 of hADAR2 amino acid sequence, or a corresponding position in a homologous or orthologous ADAR protein: (iii) the first domain comprises an amino acid sequence having a mutation at one or more positions selected from the group consisting of V351, S370, T375, P462, S486, E488, N597 and E1008 of hADAR2 amino acid sequence, or a corresponding position in a homologous or orthologous ADAR protein;
or (iv) the first domain comprises an amino acid sequence having a mutation at position Y132 of human APOBEC3A amino acid sequence, or a corresponding position in a homologous or orthologous APOBEC protein.

17. The system of claim 16, wherein; (i) the first domain comprises an amino acid sequence having a mutation at one or more positions selected from the group consisting of T375Q, E448Q and E488Q of hADAR2 amino acid sequence, or a corresponding position in a homologous or orthologous ADAR protein: (ii) the first domain comprises an amino acid sequence having a mutation at one or more positions selected from the group consisting of T339, R348, A353, V351, V355, T375, K376, E396, S397, E438, F442, H443, L444, Y445, T448, C451, R455, S486, Q488, R510, I520, V525, P539, G593, K594 and E1008 of hADAR2 amino acid sequence, or a corresponding position in a homologous or orthologous ADAR protein; (iii) the first domain comprises an amino acid sequence having a mutation at one or more positions selected from the group consisting of V351G, S370C, T375S, P462A, S486A, E488Q, N597I and E1008Q of hADAR2 amino acid sequence, or a corresponding position in a homologous or orthologous ADAR protein: or (iv) the first domain comprises an amino acid sequence having a mutation at position Y132R or Y132D of human APOBEC3A amino acid sequence, or a corresponding position in a homologous or orthologous APOBEC protein.

18. (canceled)

19. (canceled)

20. (canceled)

21. (canceled)

22. (canceled)

23. (canceled)

24. (canceled)

25. The system of claim 1, wherein the polypeptide comprises a linker between the first domain and the second domain: the polypeptide further comprises a nuclear export signal (NES) sequence, optionally the NES sequence is located between the first and the second domain: the polypeptide further comprises a FLAG octapeptide: or the polypeptide lacks nuclease activity.

26. (canceled)

27. (canceled)

28. (canceled)

29. (canceled)

30. The system of claim 1, wherein the oligonucleotide is double-stranded and comprises at least one 3′-single stranded overhang or a blunt end.

31. (canceled)

32. (canceled)

33. The system of claim 1, wherein the oligonucleotide comprises a double-stranded region of at least 19 base-pairs, optionally the oligonucleotide comprises a double-stranded region of at least 25 base-pairs.

34. (canceled)

35. The system of claim 1, wherein the oligonucleotide is double-stranded and comprises a strand having a nucleotide sequence substantially complementary to a target RNA and wherein said strand comprises a mismatch with the target RNA at position 10, counting from 5′-end of said strand.

36. The system of claim 1, wherein the oligonucleotide is double-stranded and comprises a strand having a nucleotide sequence substantially complementary to a target RNA and wherein: (i) said strand comprises a C at position 21, 22, 23, 24, 25, 26, 27 or 28, counting from 5′-end of said strand, and the strand comprises an A:C mismatch with the target RNA at position 21, 22, 23, 24, 25, 26, 27 or 28, counting from 5′-end of said strand: or (ii) said strand comprises a C at position 4, 5, 6, 7, 8, 9 or 10, counting from 3′-end of said strand, and the strand comprises an A:C mismatch with the target RNA at position 4, 5, 6, 7, 8, 9 or 10, counting from 3′-end of said strand.

37. The system of claim 36, wherein the oligonucleotide is double-stranded and comprises a strand having a nucleotide sequence substantially complementary to a target RNA and wherein: (i) said strand comprises a C at position 25, counting from 5′-end of said strand, and the strand comprises an A:C mismatch with the target RNA at position 25, counting from 5′-end of said strand: or (ii) said strand comprises a C at position 7, counting from 3′-end of said strand, and the strand comprises an A:C mismatch with the target RNA at position 7, counting from 3′-end of said strand

38. (canceled)

39. (canceled)

40. The system of claim 1, wherein the oligonucleotide is double-stranded and comprises a strand having a nucleotide sequence substantially complementary to a target RNA, wherein the target RNA forms a loop structure when hybridized to said strand, and wherein said loop structure comprises a single-stranded C nucleotide, optionally said loop structure is 5 to 20 nucleotides in length.

41. (canceled)

42. The system of claim 40, wherein said single-stranded C nucleotide is at position 6, 7, 8, 9, 10, 11, 12, counting from 5′-end of the loop structure, or said loop structure is in form of hairpin and said C nucleotide is present in a single stranded region of the hairpin.

43. (canceled)

44. The system of claim 40, wherein: (i) said loop structure is at a position opposite of position 8, 9, 10, 11, 12 or 13, counting from the 3′-end or 5′-end of said strand having a nucleotide sequence substantially complementary to the target RNA;
or (ii) said loop structure is at a position opposite of position 8, 9, 10, 11, 12 or 13, counting from the 5′-end of said strand having a nucleotide sequence substantially complementary to the target RNA.

45. (canceled)

46. (canceled)

47. (canceled)

48. (canceled)

49. (canceled)

50. (canceled)

51. (canceled)

52. A cell comprising a polypeptide or a nucleic acid encoding a polypeptide of claim 1.

53. (canceled)

54. A method of modifying a target RNA, the method comprising contacting the target RNA with the system of claim 1.

55.-63. (canceled)
